(19) United States
(12) Patent Application Publication
(10) Pub. No.: US 2025/0327126 A1
(43) Pub. Date: Oct. 23, 2025

(54) Methods, Systems, and Compositions for Diagnosing Pancreatic Transplant Rejection

(71) Applicants: Transplant Genomics, Inc., Framingham, MA (US); Hospital Clinic Barcelona, Barcelona (ES); Fundació De Recerca Clinic Barcelona-Institut D’investigacions Biomèdiques August Pi I Sunyer, Barcelona (ES); Eurofins Genoma, Roma RM (IT)

(72) Inventors: Juston Weems, Lenexa, KS (US); Ettore Cotroneo, Roma (IT); Pedro Ventura-Aguiar, Barcelona (ES); Maria Jose Ramirez-Bajo, Barcelona (ES); Fritz Diekmann, Barcelona (ES); Steve Kleiboeker, Lenexa, KS (US)

(73) Assignees: Transplant Genomics, Inc., Framingham, MA (US); Hospital Clinic Barcelona, Barcelona (ES); Fundació De Recerca Clinic Barcelona-Institut D’investigacions Biomèdiques August Pi I Sunyer, Barcelona (ES); Eurofins Genoma, Roma RM (IT)

(21) Appl. No.: 19/110,541

(22) PCT Filed: Sep. 11, 2023
(86) PCT No.: PCT/US2023/073885

§ 371 Date: Mar. 11, 2025

Related U.S. Application Data
(60) Provisional application No. 63/375,275, filed on Sep. 12, 2022.

(51) Int. Cl. C12Q 1/6883; C12Q 1/6851; C12Q 1/6881

(52) U.S. Cl. CPC C12Q 1/6883; C12Q 1/6881; C12Q 1/6851; C12Q 2600/112; C12Q 2600/158

(57) ABSTRACT

Described herein are methods, compositions, and systems useful for detecting transplant rejection and associated abnormal conditions in solid organ transplant recipients, such as pancreatic transplant recipients, pancreatic and kidney transplant recipients, and simultaneous pancreatic and kidney transplant recipients. Methods described herein may involve combined assessment of blood gene expression profiles from an assessment of particular, related mRNA transcript levels and donor-derived cell-free nucleic acids (dd-cfDNA) or each an independent assessment of the mRNA transcript level as well as an independent assessment of the dd-cfDNA. Genes that correlate with pancreatic transplant rejection in simultaneous pancreatic and kidney transplant recipients are also disclosed.

### FIELD

Described herein are methods, compositions, and systems useful for detecting transplant rejection and associated abnormal conditions in solid organ transplant recipients, such as pancreatic transplant recipients, pancreatic and kidney transplant recipients, and simultaneous pancreatic and kidney transplant recipients. Methods described herein may involve combined assessment of blood gene expression profiles from an assessment of particular, related mRNA transcript levels and donor-derived cell-free nucleic acids (dd-cfDNA) or each an independent assessment of the mRNA transcript level as well as an independent assessment of the dd-cfDNA. Genes that correlate with pancreatic transplant rejection in simultaneous pancreatic and kidney transplant recipients are also disclosed.

### BACKGROUND

Rejection in a solid organ transplant recipient, such as a pancreatic transplant recipient, pancreatic and kidney transplant recipient, or a simultaneous pancreatic and kidney transplant recipient, can manifest as clinical acute rejection, detectable by phenotypic markers, or a subclinical acute rejection, for example, which may not be detectable with commonly used clinical markers. Subclinical acute rejection, for example, is associated with worse clinical outcomes, including higher risk of subsequent clinical acute rejection, de novo donor-specific antibody (DSA) formation and associated antibody-mediated rejection, and graft fibrosis. Several clinical trials suggest that treating subclinical rejection improves outcomes. Monitoring patients for subclinical rejection typically involves serial surveillance biopsies to detect the rejection. However, despite clinical evidence, only about half of high-volume transplant programs in the United States perform surveillance biopsies. Hence, noninvasive methods of assessing the status of a solid organ transplant are needed.

In addition, rejection, such as acute or subacute rejection, may be T cell mediated (cellular mediated rejection) or it may be antibody-mediated, or a combination of the two, which may lead to different treatments, depending on which is detected. Improved screening of both clinical and subclinical acute rejection in solid organ transplant recipients may also assist in detecting the primary cause of the rejection-cellular mediated or antibody mediated or both, which may assist in determining the best treatments in response to the rejection.

### SUMMARY

As indicated above, there is a need for improved methods, systems, and compositions for detecting rejection in solid organ transplant recipients, such a in a single organ transplant recipient, such as pancreas, or in a multiple organ transplant recipient, such as pancreas and kidney (e.g., pancreas after kidney or PAK), and simultaneous pancreas and kidney (SPK) recipients, as an alternative to surveillance biopsies and/or for cause biopsies. The present disclosure relates to methods for distinguishing rejection from non-rejection in solid organ transplant recipients, in some cases those showing no clinical symptoms of rejection, and in other cases in those showing clinical signs of rejection. Methods herein include determining both the level of donor-derived, cell-free DNA (dd-cfDNA), and in some cases, comparing the level to that of a pre-determined threshold in which levels above the threshold indicate possible rejection and levels below the threshold indicate possible non-rejection. Methods herein also include determining the expression level of at least one mRNA transcript in a sample from a solid organ transplant recipient, such as a blood, plasma serum or urine sample, such as at least one mRNA transcript of a gene listed in Table A and/or Table 3 below. For example, the pattern of mRNA expression of a recipient can be compared to those of recipients with rejection and recipients with non-rejection in order to determine likelihood of rejection or likelihood of non-rejection based on the expression level. In some cases, methods herein comprise determining both dd-cfDNA level and the expression level of at least one mRNA transcript. In some cases, the recipient does not show clinical signs of rejection. In some cases, the methods help to distinguish cellular mediated rejection from antibody mediated rejection in that the level of dd-cfDNA and the expression level of the at least one mRNA transcript tend to correlate more with one of these two types of rejection over the other, thus providing a more precise determination of the rejection status of a recipient. The present disclosure also relates to methods of distinguishing rejection from non-rejection in a subject that shows signs of clinical rejection, by determining the level of dd-cfDNA. The present disclosure also relates to methods of distinguishing rejection from non-rejection in a subject that does not show signs of clinical rejection, by determining the level of dd-cfDNA. Methods herein also relate to determining the mRNA expression level of one or more genes whose levels were found to be higher in cases of rejection than in cases of non-rejection.

Some exemplary methods herein include, for example, methods of distinguishing rejection from non-rejection in a pancreas transplant recipient, or in a pancreas and kidney transplant recipient, such as a PAK or SPK recipient, comprising (a) obtaining a blood, plasma, or serum sample from the pancreas transplant recipient; (b) obtaining cell-free DNA (cfDNA) and mRNA from the sample; (c) determining (i) the level of donor derived cell-free DNA (dd-cfDNA) in the cfDNA and (ii) the expression level of at least one mRNA transcript, wherein the at least one mRNA transcript shows significantly different expression levels in pancreatic transplant rejection compared to pancreatic transplant non-rejection subjects; and (d) distinguishing rejection from non-rejection in the recipient based upon results from both the dd-cfDNA and the expression level of at least one mRNA transcript. Rejection in the recipient is indicated by either or both of (i) a level of dd-cfDNA at or above a pre-determined threshold value, and/or (ii) result of a trained algorithm based on the expression level of the at least one mRNA transcript indicating rejection or non-rejection, wherein the algorithm compares the expression profile of the at least one mRNA transcript of the recipient to the expression profile of transplant subject with and without rejection. In some cases, the recipient is a pancreatic and kidney transplant recipient, such as a pancreas after kidney transplant recipient or a simultaneous pancreas and kidney transplant recipient. In some cases, rejection in the recipient is indicated by a pre-determined threshold value of dd-cfDNA of ≥0.5%, ≥0.6%, ≥0.7%, ≥0.8%, ≥0.9%, ≥1%, ≥1.2%, ≥1.5%, or ≥2%. In some cases, rejection in the recipient is indicated by a pre-determined threshold value of dd-cfDNA of ≥0.7%, optionally wherein determining the dd-cfDNA level utilizes data from recipient genotype information. In some cases, rejection in the recipient is indicated by a pre-determined threshold value of dd-cfDNA of ≥% 1.0 or >1.0%, optionally wherein determining the dd-cfDNA level utilizes data from recipient genotype information. In some cases, rejection in the recipient is indicated by a pre-determined threshold value of dd-cfDNA of >% 1.0, optionally wherein determining the dd-cfDNA level utilizes data from recipient genotype information. In some cases, rejection in the recipient is indicated by a pre-determined threshold value of dd-cfDNA of ≥1.0%. In some cases, the methods comprise determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample. In some cases, the at least one mRNA transcript comprises one or more of the mRNA transcripts of Table A or Table 3. In some cases, the at least one mRNA transcript comprises one or more of the mRNA transcripts of Table A, such as 2-120, 5-120, 10-120, 50-120, 80-120, 5-50, 10-50, 50-100, or all of the mRNA transcripts of Table A or Table 3. In some cases, a method or both methods described herein are performed before the transplant operation and at least one hour, twenty four hours, seven days after transplantation to establish a baseline. In some cases, the method is performed at least one month, at least two months, at least three months, at least six months, or at least one year after transplantation. In some cases, the method is performed from one month to twelve months after transplantation, such as from one month to three months, or from one month to six months after transplantation. In some cases, the expression level of the at least one mRNA transcript is determined by reverse transcription PCR (RT-PCR) (such as quantitative RT-PCR), hybridization to an array, or next generation sequencing. In some cases, the dd-cfDNA level is determined by whole genome sequencing. In some cases, determining the dd-cfDNA level comprises comparison of recipient and donor genotype information, and in other cases the dd-cfDNA is determined without comparison to donor genotype information. In some cases, the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects. In some cases, the method is capable of further distinguishing likelihood of acute cellular rejection from antibody-mediated rejection, wherein the dd-cfDNA level indicates presence or absence of antibody-mediated rejection, and/or wherein the level of the at least one mRNA transcript indicates presence or absence of acute cellular rejection. The dd-cfDNA presents a positive predictive value (PPV) of at least 80%, such as at least 83% or at least 86% and/or a specificity of at least 85%, or at least 90%, or at least 93% for rejection. In some cases, rejection in the recipient is indicated by result of a trained algorithm based on the expression level of the at least one mRNA transcript indicating rejection or non-rejection, wherein the algorithm compares the expression profile of the at least one mRNA transcript of the recipient to the expression profile of pancreatic transplant subjects with and without rejection, optionally wherein the algorithm has a negative predictive value of at least 80%, such as at least 83% or at least 86% and specificity of at least 75%, such as at least 80% or at least 83%. In some cases, the method has a negative predictive value (NPV) of at least 80%, at least 85%, at least 87%, at least 88%, at least 90%, at least 92%, or at least 94% when both the level of dd-cfDNA is below the pre-determined threshold value and the result of a trained algorithm based on the expression level of the at least one mRNA transcript does not indicate rejection. In some cases, the method has a positive predictive value (PPV) of at least 80%, at least 81%, at least 82%, at least 84%, at least 86%, at least 88%, or at least 89% when both the level of dd-cfDNA is at or above the pre-determined threshold value and the result of a trained algorithm based on the expression level of the at least one mRNA transcript indicates rejection. In some cases, determining the dd-cfDNA level utilizes data from recipient genotype information and the expression level of the at least one mRNA transcript is determined by reverse-transcription PCR (RT-PCR) (such as quantitative RT-PCR). In some cases, the pre-determined threshold value of the dd-cfDNA is determined by a multivariate regression algorithm that comprises dd-cfDNA levels and expression levels of the at least one mRNA transcript in a set of transplant recipients who received the same solid organ transplant as the recipient. In some cases, the recipient has received other biomarker test results, such as from amylase, lipase, glucose, HbAlC and/or C-Peptide tests indicating presence of rejection. For example, in some cases, the recipient's serum lipase level has risen in comparison to a baseline level just prior to transplantation or in comparison to an earlier level post-transplantation, such as by at least 3-fold. In some cases, the recipient's serum amylase level has risen in comparison to a baseline level just prior to transplantation or in comparison to an earlier level post-transplantation, such as by at least 3-fold. In some cases, the recipient's fasting blood glucose test result indicates hyperglycemia (i.e., is above 120 mg/dL). In some cases, the recipient shows evidence of de novo donor specific antibodies and/or anti-glutamic acid decarboxylase antibodies (GAD). In some cases, a method herein is performed prior to or in place of a for cause biopsy, such as in a recipient displaying one or more of the above indications of rejection. Thus, in some cases, the recipient has one or more of the following characteristics: (a) at least three fold increase in serum lipase and/or serum amylase compared to baseline prior to transplantation, (b) a fasting blood glucose level of >120 mg/dL, (c) presence of donor specific antibodies, or (d) presence of anti-glutamic acid decarboxylase (GAD) antibodies, optionally wherein the method is performed in lieu of a pancreas or kidney biopsy.

Methods herein also comprise, for example, a method of distinguishing rejection from non-rejection in a pancreatic transplant recipient, the method comprising (a) obtaining a sample from the pancreatic transplant recipient; (b) obtaining mRNA from the sample; (c) determining the expression level of at least one mRNA transcript selected from the mRNA transcript of at least one gene listed in Table A or Table 3, wherein the at least one mRNA transcript shows significantly higher expression levels in pancreatic transplant rejection compared to pancreatic transplant non-rejection subjects; and (d) distinguishing rejection from non-rejection in the recipient based upon the expression level of at least one mRNA transcript, optionally wherein rejection in the recipient is indicated by result of a trained algorithm based on the expression level of the at least one mRNA transcript indicating rejection or non-rejection, wherein the algorithm compares the expression profile of the at least one mRNA transcript of the recipient to the expression profile of pancreatic transplant subjects with and without rejection. Methods herein also comprise distinguishing rejection from non-rejection in a pancreatic transplant recipient, the method comprising (a) obtaining a blood, plasma, or serum sample from the pancreatic transplant recipient; (b) obtaining cell-free DNA (cfDNA) and mRNA from the sample; (c) determining (i) the level of donor derived cell-free DNA (dd-cfDNA) in the cfDNA and (ii) the expression level of at least one mRNA transcript selected from the mRNA transcript of at least one gene listed in Table A or Table 3, wherein the at least one mRNA transcript shows significantly higher expression levels in pancreatic transplant rejection compared to pancreatic transplant non-rejection subjects; and (d) distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) the expression level of the at least one mRNA transcript. In some such cases, the recipient is a pancreatic and kidney transplant recipient, such as a pancreas after kidney transplant recipient or a simultaneous pancreas and kidney transplant recipient. In some cases, rejection in the recipient is indicated by predetermined threshold value of dd-cfDNA of ≥0.5%, ≥0.6%, ≥0.7%, ≥0.8%, ≥0.9%, ≥1%, ≥1.2%, ≥1.5%, or ≥2%. In some cases, rejection in the recipient is indicated by a pre-determined threshold value of dd-cfDNA of ≥0.7%, optionally wherein determining the dd-cfDNA level utilizes data from recipient genotype information. In some cases, rejection in the recipient is indicated by a pre-determined threshold value of dd-cfDNA of ≥1.0 or >1.0, optionally wherein determining the dd-cfDNA level utilizes data from recipient genotype information. In some cases, the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample. In some cases, the at least one mRNA transcript (a) comprises an mRNA transcript of one or more of: IGHG2, IGHG1, IGHG4, IGHA1, IGLC1, IGHG3, IGKC, IRF4, IL7R, CD96, SLAMF6, SLAMF7, GZMK, IGHM, ZAP70, CD3E, CD79A, CXCR6, CD3D, MIR155HG, CTSW, SLA, IL2RG, CXCR4, ISG20, IL2RB, CCL5, PRDM1, CCR5, CCR2, AOAH, HLA-DQB1, IDO1, GZMA, IKZF1, KLRB1, TNFSF8, CD8A, IL16, HLA-DRA, CD84, BTK, NKG7, SELPLG, CD45R0, PTPRC, or ITGAX; or (b) comprises a group of 2-47, 2-40, 2-30, 2-20, 2-10, 10-40, 10-20, or 20-40 mRNA transcripts listed in Table 3 or selected from IGHG2, IGHG1, IGHG4, IGHA1, IGLC1, IGHG3, IGKC, IRF4, IL7R, CD96, SLAMF6, SLAMF7, GZMK, IGHM, ZAP70, CD3E, CD79A, CXCR6, CD3D, MIR155HG, CTSW, SLA, IL2RG, CXCR4, ISG20, IL2RB, CCL5, PRDM1, CCR5, CCR2, AOAH, HLA-DQB1, IDO1, GZMA, IKZF1, KLRB1, TNFSF8, CD8A, IL16, HLA-DRA, CD84, BTK, NKG7, SELPLG, CD45R0, PTPRC, or ITGAX. In some cases, the method is performed at least one month, at least two months, at least three months, at least six months, or at least one year after transplantation. In some cases, the method is performed from one month to twelve months after transplantation, such as from one month to three months, or from one month to six months after transplantation. In some cases, the expression level of the at least one mRNA transcript is determined by reverse transcription PCR (RT-PCR) (such as quantitative RT-PCR), hybridization to an array, or next generation sequencing. In some cases, the dd-cfDNA level is determined by whole genome sequencing. In some cases, determining the dd-cfDNA level utilizes data from recipient genotype information and the expression level of the at least one mRNA transcript is determined by reverse-transcription PCR (RT-PCR) (such as quantitative RT-PCR). In some cases, the pre-determined threshold value of the dd-cfDNA is determined by a multivariate regression algorithm that comprises dd-cfDNA levels and expression levels of the at least one mRNA transcript in a set of transplant recipients who received the same solid organ transplant as the recipient. In some cases, determining the dd-cfDNA level comprises comparison of recipient and donor genotype information. In some cases, the dd-cfDNA is determined without comparison to donor genotype information. In some cases, the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects. In some cases, the method has a negative predictive value (NPV) of at least 80%, at least 85%, at least 87%, at least 88%, at least 90%, at least 92%, or at least 94% when both the level of dd-cfDNA is below the pre-determined threshold value and the result of a trained algorithm based on the expression level of the at least one mRNA transcript does not indicate rejection. In some cases, the method has a positive predictive value (PPV) of at least 80%, at least 81%, at least 82%, at least 84%, at least 86%, at least 88%, or at least 89% when both the level of dd-cfDNA is at or above the pre-determined threshold value and the result of a trained algorithm based on the expression level of the at least one mRNA transcript indicates rejection. In some cases, the recipient has received other biomarker test results, such as from amylase, lipase, glucose, HbAlC and/or C-Peptide tests indicating presence of rejection. For example, in some cases, the recipient's serum lipase level has risen in comparison to a baseline level just prior to transplantation or in comparison to an earlier level post-transplantation, such as by at least 3-fold. In some cases, the recipient's serum amylase level has risen in comparison to a baseline level just prior to transplantation or in comparison to an earlier level post-transplantation, such as by at least 3-fold. In some cases, the recipient's fasting blood glucose test result indicates hyperglycemia (i.e., is above 120 mg/dL). In some cases, the recipient shows evidence of de novo donor specific antibodies and/or anti-glutamic acid decarboxylase antibodies (GAD). In some cases, a method herein is performed prior to or in place of a for cause biopsy, such as in a recipient displaying one or more of the above indications of rejection. Thus, in some cases, the recipient has one or more of the following characteristics: (a) at least three fold increase in serum lipase and/or serum amylase compared to baseline prior to transplantation, (b) a fasting blood glucose level of >120 mg/dL, (c) presence of donor specific antibodies, or (d) presence of anti-glutamic acid decarboxylase (GAD) antibodies, optionally wherein the method is performed in lieu of a pancreas or kidney biopsy.

All publications, patents, and patent applications cited in this disclosure (either in the text or in a reference list) are incorporated by reference herein in their entireties. Further description of embodiments of the disclosure is provided in the sections that follow and in the drawings.

### BRIEF DESCRIPTION OF THE DRAWINGS

A better understanding of certain features and advantages of the embodiments described herein may be obtained by reference to the accompanying drawings, summarized below.

FIG. 1 (FIG. 1) depicts the dynamic of % dd-cfDNA after pancreas transplantation in patients with stable graft function. DO, pre-transplant; 1 h, 1 hour after reperfusion, and 24 h, 24 hours after reperfusion; 7D, 7 days after transplant; B3 and B12, biopsies for surveillance at 3 weeks and 12 months post-transplant.

FIG. 2 (FIG. 2) describes the monitoring of a pancreas graft after transplantation.

FIG. 3A-3B (FIG. 3A-3B) describes the dynamic of donor derived cfDNA in pancreas transplantation (FIG. 3A) and correlation of dd-cfDNA at 1 hour post transplant with donor demographics and cold ischemia time (FIG. 3B).

FIG. 4A-4D (FIG. 4A-4D) further describes the dynamic of donor derived cfDNA in pancreas transplantation showing several subjects (FIG. 4A), and for three representative subjects in each of FIGS. 4B, 4C, and 4D, showing antibody mediated rejection (ABMR) or T cell mediated rejection (TMR).

FIG. 5A-5B (FIG. 5A-5B) further describes the dynamic of donor derived cfDNA in pancreas transplantation (FIG. 5A), as confirmed by the Banff Classification (FIG. 5B).

FIG. 6A-6B (FIG. 6A-6B) describes the sensitivity and specificity of cfDNA (FIG. 6A) and the negative predictive value (NPV) and the positive predictive value (PPV) in cfDNA (FIG. 6B).

FIG. 7 (FIG. 7) describes transplant excellence (TX) and not transplant excellence (no TX) using the TruGraf® assay for assessing transplant graft rejection.

FIG. 8 (FIG. 8) describes TruGraf® assay in pancreas graft rejection.

FIG. 9A-9B (FIG. 9A-9B) describes TruGraf® assay in pancreas graft rejection by Banff classification with all samples (FIG. 9A) and excluding certain samples (FIG. 9B).

FIG. 10A-10B (FIG. 10A-10B) describes TruGraf® assay in pancreas graft rejection as compared to amylase (FIG. 10A) and lipase (FIG. 10B) assays.

FIG. 11 (FIG. 11) further analyzes TruGraf® assay in pancreas graft rejection as compared to lipase assay.

FIG. 12A-12B (FIG. 12A-12B) describes TruGraf® assay in pancreas graft rejection compared to lipase assay in (FIG. 12A) comparing TX/no TX TruGraf® for samples with either less than 3 times normal (<3×s/normal) lipase (left bars) or >3×s/normal lipase (right bars); or (FIG. 12B) comparing performance of TruGraf® vs lipase assay only in subjects with <3×s/normal lipase.

FIG. 13 (FIG. 13) describes TruGraf® assay in pancreas graft rejection.

FIG. 14 (FIG. 14) describes TruGraf® assay in pancreas graft rejection.

FIG. 15A-15B (FIG. 15A-15B) describes TruGraf® assay in comparison to dd-cfDNA assay in pancreas graft rejection in each of graphs of FIG. 15A and FIG. 15B.

FIG. 16A-16B (FIG. 16A-16B) describes TruGraf® assay and dd-cfDNA assay in pancreas graft rejection compared to a lipase assay, for all subjects in FIG. 16A and for subjects with <3×s/normal lipase in FIG. 16B.

FIG. 17 (FIG. 17) provides a dynamic of donor-derived cfDNA in SPK patients post transplant during the study period from Example 2. D0, pre-transplant; 1 h, 1 hour after reperfusion, and 24 h, 24 hours after reperfusion; 7D, 7 days after transplant; B3 and B12, biopsies for surveillance at 3 weeks and 12 months post-transplant. Mann-Whitney U test was used to compare group's means versus D0. **** p<0.0001.

FIG. 18A-18C (FIG. 18A-18C) provides dynamics of dd-cfDNA over time of SPK recipients. FIG. 18A shows % dd-cfDNA at the individual level in those patients with multiple samples collected. Patients without immunological events with dd-cfDNA <1% from day 7 onwards (black lines), patient without immunological events but with dd-cfDNA >1% beyond day 7 (slowly rising line), and those patients who presented an acute rejection during the first year (variably lowering and rising lines). FIG. 18B and FIG. 18C show two representative cases of the dynamic % dd-cfDNA in FIG. 18B) a recipient with subsequent increase at acute rejection episodes, and FIG. 18C) a recipient with elevated % dd-cfDNA levels despite the diagnosis and treatment for acute rejection. TCMR=T-cell Mediated rejection; ABMR=Antibody-Mediated Rejection.

FIG. 19A-19C (FIG. 19A-19C) shows a comparison of median % dd-cfDNA from Example 2, FIG. 19A) between pancreas biopsy-proven acute rejection (BPAR) and no-rejection (N-BPAR) groups, FIG. 19B) during the first 90 days post-transplant, and FIG. 19C) after the first 90 days post-transplant. Mann-Whitney U test was used to compare group's means. * P<0.037.

FIG. 20A-20D (FIG. 20A-20D) shows Trugraf® classification from Example 2, in FIG. 20A) according to the diagnosis of acute rejection in the biopsy-matched cases evaluated, and FIG. 20B) according to the Banff classification scheme. FIG. 20C shows Trugraf® discrimination ability in biopsies performed for cause in recipients of SPK. FIG. 20D) Trugraf® discrimination in patients with sub-clinical (lipase <3×s/normal) pancreas acute rejection. SPK=simultaneous kidney-pancreas transplant.

### DETAILED DESCRIPTION

### Definitions

Unless defined otherwise, all technical and scientific terms used herein have the same meaning as commonly understood by those of ordinary skill in the art to which this invention pertains. In addition, the following definitions are provided to assist the reader in the practice of the invention.

The term “or” as used herein and throughout the disclosure is intended as an inclusive “or,” meaning “and/or” unless the context expressly indicates otherwise.

The terms “a” or “the” as used herein and throughout the disclosure are intended to encompass both singular and plural, i.e., to mean “at least one,” unless the context expressly indicates otherwise.

The terms “transplantation” or a “transplant” generally refer to the transfer of tissues, cells, or a solid organ from a donor individual into a recipient individual. A donor and recipient may or may not be from the same species. Thus, for example, a human recipient may receive a solid organ from a non-human animal in some embodiments. An “allograft” further indicates a transfer of tissues, cells, or a solid organ between different individuals of the same species. In contrast, if the donor and recipient are the same individual, the graft is referred to as an “autograft.”

A “recipient” generally refers to an individual receiving a transplant, allograft, or autograft. A “recipient” herein is a human, unless expressly stated otherwise (i.e., a murine recipient or the like). The terms “individual,” “subject,” or “patient” in the context of transplantation or medical treatment generally refer interchangeably to a human receiving such a transplantation or other medical treatment, e.g., a recipient of a transplant or of other medical treatment. In some embodiments, certain analyses are performed on samples from a recipient post transplant.

As used herein, a recipient that does not have rejection, or that shows “non-rejection,” or is negative for rejection, or the like, which may also be abbreviated “TX” herein, standing for “transplant excellence,” generally signifies that the recipient does not exhibit symptoms or test results indicating organ dysfunction or rejection. Accordingly, in such recipients the transplant is considered a normal functioning transplant. A “TX” patient can have normal histology on a surveillance biopsy (e.g. no evidence of rejection), and in the context of a pancreatic transplant recipient: can have the Banff classifications set forth in the Guidelines for the Diagnosis of Antibody-Mediated Rejection in Pancreas Allografts-Updated Banff Grading Schema, American Journal of Transplantation 200:11:1792-1802 that indicate non-rejection.

In contrast, a “rejection” (also termed “non-TX,” i.e., “not transplant excellence,” herein) can be observed either clinically or subclinically, for example, such as via biomarker tests herein or via histology. The term “rejection” herein encompasses several sub-types of rejection, such as clinical or subclinical acute rejection, acute cellular rejection or T cell mediated rejection, and antibody-mediated rejection.

“Acute rejection (AR)” or “clinical acute rejection” generally refers to a condition that can occur when transplanted tissue is rejected by the recipient's immune system, which damages or destroys the transplanted tissue unless immunosuppression is achieved. T-cells, B-cells and other immune cells as well as possibly antibodies of the recipient may cause the graft cells to lyse or produce cytokines that recruit other inflammatory cells, eventually causing necrosis of allograft tissue. In some instances, AR can be diagnosed by a biopsy of the transplanted organ. AR can occur more frequently in the first three to 12 months after transplantation but there is a continued risk and incidence of AR for the first five years post-transplant and whenever a patient's immunosuppression becomes inadequate for any reason for the life of the transplant.

As used herein, the term “subclinical acute rejection” (also “subAR”) or “subclinical rejection” refers to histologically defined acute rejection (e.g. histology on a surveillance biopsy consistent with acute rejection), but without the requirement of functional deterioration. In some instances, subAR can represent the beginning or conclusion of an alloimmune infiltrate diagnosed fortuitously by protocol sampling, and some episodes of clinical rejection may actually represent subAR with an alternative cause of functional decline. A subAR subject can have normal and stable organ function. SubAR can be distinguished from acute rejection. as acute rejection requires acute renal impairment. The differences between subAR and acute rejection can involve real quantitative differences of cortex affected, qualitative differences, or an increased ability of the allograft to withstand immune injury (‘accommodation’). SubAR is often diagnosed only on biopsies taken as per protocol at a fixed time after transplantation, rather than driven by clinical indication, and is accordingly difficult to detect by traditional function measurements.

Subclinical acute rejection may comprise “acute cellular rejection,” which is also called “T cell mediated rejection” or “cell mediated rejection,” abbreviated TMR or TCMR. Subclinical acute rejection may also or alternatively comprise “antibody mediated rejection, which is abbreviated “ABMR” or “AMR.” T cell mediated rejection (“TMR” or “TCMR”), for example, may be associated with an increase in activity of certain T cell populations in the vicinity of the transplanted organ or tissue, or markers for such cells. Antibody-mediated rejection, for example, may be associated with injury to the transplanted tissue or organ, and may be characterized by the production of IgG antibodies against the transplanted tissue, such as anti-HLA antibodies.

In some experiments herein, recipients whose outcomes were known based on biopsy had their samples further analyzed for example to assess donor derived cell free DNA or expression of certain genes or mRNA transcripts, or the like. In some cases, a recipient sample so tested may be “BPAR,” which stands for biopsy proven acute rejection,” or in the case of a pancreas biopsy, may be “P-BPAR,” standing for pancreas biopsy proven acute rejection.”

In some experiments herein, a subject receiving a pancreas transplantation has also received a kidney transplantation. In some embodiments, one transplantation may follow the other in two different procedures, such as pancreatic transplantation after kidney transplantation, or “pancreas after kidney,” abbreviated “PAK.” In other cases, both transplantations may be done in one procedure, called “simultaneous pancreas kidney” transplantation, or “SPK.”

A “likelihood” of a particular type of subclinical rejection may be obtained in methods herein. For example, certain biomarker tests, when positive, tend to correlate with a particular type of subclinical rejection such as antibody-mediated rejection or acute cellular rejection over another type of rejection, thus indicating that the subject is likely to have a particular type of rejection over another.

Some biomarker tests described herein, such as based on dd-cfDNA or gene expression, are associated with a “positive predictive value” or “PPV”, for example, in some cases above a certain percentage. A PPV is the probability that a test result indicating an abnormality such as transplant rejection actually has the abnormal phenotype such as rejection. Some biomarker tests herein are associated with a “negative predictive value” or “NPV,” for example, in some cases above a certain percentage. An NPV is the probability that a test result indicating that a subject is normal or does not have a phenotype such as rejection actually predicts that the subject is normal and does not have rejection. Thus, for example, a test with a high NPV might be used to rule out certain abnormalities such as rejection. A test with a high PPV might be used to detect the presence of an abnormality such as rejection.

As used herein, in performing the methods, “obtaining a sample” includes obtaining a sample directly or indirectly. In some embodiments, the sample is taken from the subject by the same party (e.g. a testing laboratory) that subsequently acquires biomarker data from the sample. In some embodiments, the sample is received (e.g. by a testing laboratory) from another entity that collected it from the subject (e.g. a physician, nurse, phlebotomist, or medical caregiver). In some embodiments, the sample is taken from the subject by a medical professional under direction of a separate entity (e.g. a testing laboratory) and subsequently provided to said entity (e.g. the testing laboratory). In some embodiments, the sample is taken by the subject or the subject's caregiver at home and subsequently provided to the party that acquires biomarker data from the sample (e.g. a testing laboratory). As used herein, when a method herein is said to be conducted at a particular time, such as a specific time after transplantation (e.g., 1 week, 1 month, etc. following transplantation), where there is a delay between the time that the sample was taken from the recipient and when the dd-cfDNA and/or mRNA transcript expression data were obtained, the method is said to be conducted at the time that the sample was taken from the recipient, since the results reflect the state of the recipient at that point in time.

As used in the methods herein, the term “dd-cfDNA” refers to the amount of donor derived cell free DNA obtained from the cell free DNA (cfDNA) in the sample.

As used in methods herein, the term “mRNA transcript” indicates an mRNA obtained from transcription of a particular gene, and includes full length and non-full length transcripts and transcripts that result from alternative splicing. Thus, each “mRNA transcript” herein is from a different gene, and a reference to two or more mRNA transcripts, or, for example to 50 or 100 mRNA transcripts, herein means the mRNA transcripts of two or more genes or of 50 or 100 genes. An “mRNA transcript” is not necessarily a single RNA molecule. For example, due to degradation of RNA in a recipient sample, an original mRNA transcript for a gene may be degraded into multiple RNA molecules that cover the length of the transcribed coding region. But an “mRNA transcript” includes sufficient transcription of the gene coding region to be uniquely identified as belonging to the particular, transcribed gene, and thus, to be a marker of the level of expression of that gene.

The term “significantly different” in the methods herein, i.e. in referring to genes whose mRNA transcripts show changes in expression levels in rejection vs. non-rejection subjects, means statistically significantly different, such as through a T-test and an associated P value that indicates statistical significance. Similarly, if other mRNA transcripts show changes in expression levels that are “not significantly different,” the changes are not statistically significantly different.

A “biopsy” generally refers to a specimen obtained from a living patient for diagnostic or prognostic evaluation. A “surveillance biopsy” for example may be performed following a transplant to look for evidence of rejection or non-rejection, and it may be performed, for example, as a matter of course after a period of time post-transplantation regardless of the phenotype of the recipient. In contrast, a biopsy performed “for cause” indicates that the recipient was displaying some symptom or phenotype associated with rejection, thus prompting the biopsy.

The term “treatment,” for example, for a transplant recipient, includes medical management strategies such as active surveillance, which may include diagnostic or biopsy assays to assess likelihood of rejection, as well as therapeutic treatment, for example, with drugs intended to suppress rejection or promote functioning of the transplanted organ, such as immunosuppressants. Further discussion of treatments is provided below.

Additional definitions of particular terms are provided in the sections that follow.

Methods of Distinguishing Rejection from Non-Rejection

The incidence of acute rejection (both T cell mediated and antibody mediated) for pancreatic transplants is about 20-35% of all recipients, with about 10% of all recipients having antibody mediated rejection. The present disclosure relates to methods capable of distinguishing rejection from non-rejection in a solid organ transplant recipient that, in some embodiments, combine determination of the level of donor-derived, cell free DNA (dd-cfDNA) in a sample from the recipient and/or determining the expression level of at least one mRNA transcript in the sample. Where both assays are performed, the methods comprise analyzing results of both assays. In some embodiments, the method of distinguishing rejection from non-rejection comprises determining the level of donor-derived, cell free DNA in a sample from the recipient and analyzing the results. In some embodiments, the method of distinguishing rejection from non-rejection comprises determining the expression level of at least one mRNA transcript in the same or a different sample from the recipient and analyzing the results. Certain methods herein comprise: obtaining a sample from the solid organ transplant recipient; obtaining cell-free DNA (cfDNA) and mRNA from the sample; determining (i) the level of donor derived cell-free DNA (dd-cfDNA) in the cfDNA and (ii) the expression level of at least one mRNA transcript, wherein the at least one mRNA transcript shows significantly different expression levels in pancreatic transplant rejection compared to pancreatic transplant non-rejection subjects; and distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) expression level of the at least one mRNA transcript or a result of an algorithm based on the expression level indicating rejection. In some embodiments, the transplant recipient has received a pancreatic transplant. In some embodiments, the recipient has received both a pancreatic and a kidney transplant. Such transplants may, in some cases, be conducted simultaneously, i.e. simultaneous pancreas and kidney (SPK), while in other cases, they may be conducted sequentially, such as pancreas after kidney (PAK). In some cases, the pre-determined threshold for dd-cfDNA is 1.0%, such that a level at or above 1.0%, or alternatively above 1.0% indicates rejection. In some cases, the expression level of the at least one mRNA indicates pancreatic transplant rejection.

### Exemplary Samples

The methods in some embodiments may be conducted on a single sample from the recipient, for instance, a blood, serum, plasma, urine, or tissue sample, or a sample obtained by a non-invasive, minimally-invasive, or invasive procedure as discussed below. This single sample may be used to determine the level of dd-cfDNA and the expression level of at least one mRNA transcript if both assays are used. In some embodiments, the dd-cfDNA and mRNA transcript information are obtained from a single sample from the recipient. Such a “single sample” means herein a sample that is obtained from the recipient at one time, such as during one blood draw or phlebotomy appointment or during one other diagnostic or medical appointment. Accordingly, the “single sample” is not required to be present in the same sample container, but instead is merely drawn from the patient at the same time, during the context of one diagnostic or medical appointment. In some cases, such a “single sample” comprises two separate blood draws done in one visit or appointment, for example, placed into separate containers. In some cases, a “single sample” is first drawn and stored in a single container, and then is later split into multiple containers. For example, in either of these cases, different stabilizers may be used in the different containers, each compatible with the later dd-cfDNA or mRNA transcript assays. In other cases, the dd-cfDNA and mRNA transcript information is obtained on different samples from the subject, such as obtained at roughly the same time, but of different types (e.g., blood draw and a tissue sample), or is obtained on different samples taken from the subject at roughly the same time, but in different visits, or is obtained on different samples taken from the subject at different times.

In some embodiments, the sample is obtained from a non-invasive procedure, such as a throat swab, buccal swab, bronchial lavage, urine collection, skin or epidermal scraping, feces collection, menses collection, or semen collection. In other cases, a minimally-invasive procedure may be used such as a blood draw, e.g., by venipuncture methods. In other cases, a sample may be obtained by an invasive procedure such as a biopsy, alveolar or pulmonary lavage, or needle aspiration. In other cases, a sample of capillary blood could be either self-collected or collected by a caregiver or healthcare provider.

In some embodiments, the sample is a blood, serum, or plasma sample. A “blood” sample, herein refers to whole blood or fractions thereof, including plasma, lymphocytes, peripheral blood lymphocytes (PBLs), peripheral blood mononuclear cells (PBMCs), serum, T cells, B Cells, CD3 cells, CD8 cells, CD4 cells, or other immune cells. In some embodiments, it is a whole blood sample. Other samples that can be analyzed include urine, feces, saliva, and tissue from a biopsy. However, a sample may be any material containing tissues, cells, nucleic acids, genes, gene fragments, expression products, polypeptides, exosomes, gene expression products, or gene expression product fragments of a transplant recipient to be tested.

In some embodiments, a whole blood sample drawn from the recipient for analysis according to the methods herein may be, for example, 10 mL or less, 8 mL or less, 7 mL or less, 6 mL or less, or 5 mL or less. In some embodiments, a blood sample may be 6 mL or less. A blood sample may be obtained by any method, preferably a minimally-invasive method such as a blood draw or fingerstick or dried blood spot (DBS), or a self-sampling device like Tasso or TAPII. The sample may be obtained by venipuncture or fingerstick via lancet device or via capillary blood collection device. Some or all of a sample obtained from a recipient may then be used in the methods. In some embodiments, multiple samples may be obtained by the methods herein to ensure a sufficient amount of biological material. In some cases, methods herein may be performed on more than one recipient's sample, i.e., on pooled samples, then deconvoluted to determine whether any of the samples indicate rejection.

### Exemplary Solid Organ Transplant Recipients

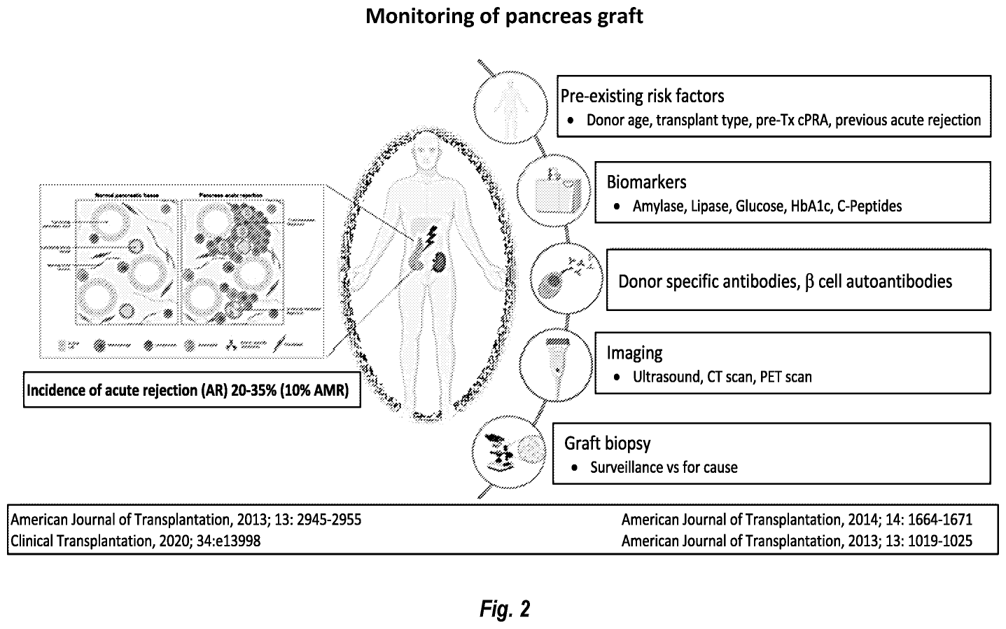

A solid organ transplant recipient may be a recipient of a solid organ or a fragment of a solid organ such as a pancreas. FIG. 2 shows the monitoring of the recipient of a pancreatic transplant. Recipients herein are humans unless specifically stated to be a different animal, such as a non-human primate (e.g., ape, monkey, chimpanzee), a domestic animal such as a cat, dog, or rabbit, or a livestock animal such as a goat, horse, cow, pig, or sheep, or a laboratory animal such as a rodent, mouse, SCID mouse, rat, guinea pig, etc.

The donor organ, tissue, or cells may be derived from a subject who has certain similarities or compatibilities with the recipient subject. For example, the donor organ, tissue, or cells may be derived from a donor subject who is age-matched, ethnicity-matched, gender-matched, blood-type compatible, or HLA-type compatible with the recipient subject. In some circumstances, the donor organ, tissue, or cells may be derived from a donor subject that has one or more mismatches in age, ethnicity, gender, blood-type, or HLA markers with the transplant recipient due to organ availability. The organ may be derived from a living or deceased donor.

In various embodiments, recipients have undergone an organ transplant within one hour, 6 hours, 12 hours, 1 day, 2 days, 3 days, 4 days, 5 days, 10 days, 15 days, 20 days, 25 days, 1 month, 2 months, 3 months, 4 months, 5 months, 6 months, 7 months, 9 months, 11 months, 1 year, 2 years, 4 years, 5 years, 10 years, 15 years, 20 years or longer of prior to being assessed by a method herein or both methods described. In some embodiments, the methods are performed at least 1 month post transplantation, such as at least 3 months post transplantation, or at least 12 months post transplantation, such as 1-3 months, 1-6 months, 3-6 months, 1-2 months, or 6-12 months, or 12-24 months post transplantation.

In some embodiments, the recipient is undergoing a treatment regimen, or being evaluated for a treatment regimen, such as immunosuppressive therapy, to inhibit rejection or to reduce at least one symptom of rejection. However, in some instances, the recipient is not undergoing a treatment regimen such as immunosuppressant therapy. In some embodiments, the subject is receiving a standard of care immunosuppressant therapy regimen for the type of solid organ transplant received. In some embodiments, the recipient has not received a biopsy, such as a surveillance biopsy prior to assessment via a method herein.

In some embodiments, the recipient has received at least one immunosuppressive drug, and, if the result of the one or both methods indicates that the recipient has clinical or subclinical acute rejection, the method comprises increasing the frequency or dosage of the at least one immunosuppressant drug, administering a further immunosuppressant drug, or administering a different immunosuppressive drug to the recipient. In some cases, if the method indicates that the recipient has clinical or subclinical acute rejection, following such adjustment of immunosuppressant therapy, the method is repeated to assess the effect of such therapy adjustment, for instance, after 1 week, 2 weeks, 1 month, 2 months, 3 months, 6 months or one year following the adjustment in the therapy. In some cases, if the method indicates that the recipient has clinical or subclinical acute rejection, a surveillance biopsy is ordered for the recipient, optionally, along with or prior to an adjustment in immunosuppressive therapy, such as increasing the frequency or dosage of the at least one immunosuppressant drug, administering a further immunosuppressant drug, or administering a different immunosuppressive drug to the recipient. In some cases, if the method or methods show that the patient is stable, the immunosuppressant drug may be decreased.

In some cases, methods herein are performed every 1 month, 2 months, 3 months, 6 months, or year following a transplant procedure, for example. In some cases, they are performed every 2 months. In some cases, every 3 months. In some cases, every 6 months. In some cases, the frequency depends on the test results. Thus, for example, in some cases methods herein may be performed with increased frequency if one or both results is positive, for instance, if treatment is subsequently adjusted.

In some embodiments, the recipient may have undergone other biomarker testing prior to conducting a method herein. For example, in the case of a pancreatic transplant recipient, the levels of amylase, lipase, glucose, HbAlC or C-Peptide may have been determined. In some cases, the recipient may have a lipase test result indicating rejection. In some cases, the recipient may have an amylase test result indicating rejection. For example, a lipase test may be performed to determine whether lipase is above or below a threshold of 156 U/L. An amylase test may be performed to determine whether amylase is above or below a threshold of 312 U/L. In some cases, a recipient may have a lipase level that is less than three times the normal level (<3 ns/normal). In other cases, a recipient may have a lipase level or amylase level that has risen, such as by at least 3-fold following transplantation.

In some cases, a transplant recipient assessed in methods herein may have results from parameters such as those above indicating normal organ function, while in other cases, the recipient may have results indicating impairment in organ function or graft failure. For example, in some cases, a recipient may present an “acute dysfunction no rejection (ADNR)” phenotype, in which the subject shows symptoms of or biomarkers associated with dysfunction of the transplanted organ, but does not show symptoms or biomarkers associated with rejection.

In some cases, the recipient has received other biomarker test results, such as from amylase, lipase, glucose, HbAlC and/or C-Peptide tests indicating presence of rejection. For example, in some cases, the recipient's serum lipase level has risen in comparison to a baseline level just prior to transplantation or in comparison to an earlier level post-transplantation, such as by at least 3-fold. In some cases, the recipient's serum amylase level has risen in comparison to a baseline level just prior to transplantation or in comparison to an earlier level post-transplantation, such as by at least 3-fold. In some cases, the recipient's fasting blood glucose test result indicates hyperglycemia (i.e., is above 120 mg/dL). In some cases, the recipient shows evidence of de novo donor specific antibodies and/or anti-glutamic acid decarboxylase antibodies (GAD). In some cases, a method herein is performed prior to or in place of a for cause biopsy, such as in a recipient displaying one or more of the above indications of rejection. For example, physicians reviewing such test results in the past may perform a for cause biopsy in such circumstances, in order to determine if rejection is present and to accordingly prescribe or adjust immunosuppressive treatment for the recipient. In some embodiments herein, a dd-cfDNA and/or mRNA expression level method as described herein is performed prior to or in place of such a for cause biopsy in such recipients. In some cases, a method as described herein is performed in place of a surveillance biopsy in a recipient.

mRNA Expression Profiles

Methods herein, for example, comprise obtaining mRNA from the recipient sample and determining the expression level of at least one mRNA transcript or a result of an algorithm based on the expression level and determining whether the expression level or the algorithm result indicates a likelihood of rejection for the recipient. In some embodiments, the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample. In some cases, the at least one mRNA transcript comprises mRNA transcripts of one or more of the genes provided in Table A below. In some cases, the at least one mRNA transcript comprises 2-120, 5-120, 10-120, 50-120, 80-120, 2-128, 5-128, 10-128, 50-128, 80-128, 5-50, 10-50, 50-100, or all of the mRNA transcripts of the genes of Table A. In some embodiments, the at least one mRNA transcript is chosen from a group consisting of 2-120, 5-120, 10-120, 50-120, 80-120, 2-128, 5-128, 10-128, 50-128, 80-128, 5-50, 10-50, 50-100, or all of the mRNA transcripts of the genes of Table A. In some cases, the at least one mRNA transcript consists of 2-120, 5-120, 10-120, 50-120, 80-120, 2-128, 5-128, 10-128, 50-128, 80-128, 5-50, 10-50, 50-100, or all of the mRNA transcripts of the genes of Table A. Furthermore, in some embodiments, the at least one mRNA transcript comprises at least one mRNA that co-expresses with at least one gene listed in Table A, or that is found in the same biological or cell signaling pathway as a gene listed in Table A herein. As noted above, the term “mRNA transcript” as used herein indicates an mRNA obtained from a gene. Thus, each “mRNA transcript” herein is from a different gene, and a reference to two or more mRNA transcripts herein means the mRNA transcripts of two or more genes. Thus, if 2-150 mRNA transcripts are assayed herein, the mRNA transcripts are assayed to determine the expression at the RNA level of 2-150 different genes.

In some embodiments, the at least one mRNA transcript is chosen from a group consisting of 2-120, 5-120, 10-120, 50-120, 80-120, 2-128, 5-128, 10-128, 50-128, 80-128, 5-50, 10-50, 50-100, or all of the mRNA transcripts of the genes of Table A (i.e., mRNA transcripts of the genes listed in Table A) and at least one reference mRNA transcript. In some embodiments, the at least one mRNA transcript consists of 2-120, 5-120, 10-120, 50-120, 80-120, 2-128, 5-128, 10-128, 50-128, 80-128, 5-50, 10-50, 50-100, or all of the mRNA transcripts of the genes of Table A and at least one reference mRNA transcript or other reference RNA (such as a ribosomal RNA or other non-mRNA molecule). In such cases, the reference mRNA transcript or other reference RNA is not expected to significantly differ in expression between a sample from a patient with rejection and one without rejection. An example of such a reference mRNA transcript is the mRNA of a so-called housekeeping gene, for instance. Examples include, for instance, one or more of ACTB, GAPDH, and YWHAE. In other cases, one or more of B2M, UBC, HPRT1, TTC5, C2orf44, or Chr3 could also act as a reference gene. In some embodiments, mRNA transcripts of a reference gene or genes are used to normalize the mRNA levels in the sample as a whole prior to analysis. In other embodiments, mRNA levels are normalized against the overall mRNA levels found in the sample. Normalization, for example, may help to control for the quality of the RNA of a sample, or the amount of the RNA of the recipient sample that is obtained.

In some embodiments, the at least one mRNA transcript whose expression level is assessed in the methods is chosen as an mRNA transcript whose expression significantly differs between solid organ transplant recipients with rejection compared to those without rejection. For example, the expression level of some mRNA transcripts may increase in the event of a rejection. In contrast, the expression level of some mRNA transcripts may decrease in the event of a rejection. In some cases, all of the assessed mRNA transcripts show an increase in expression level in the event of a rejection. In some cases, all of the assessed mRNA transcripts show a decrease in expression level in the event of a rejection. In yet other cases, some of the mRNA transcripts show an increase in expression levels in the event of a rejection, while others decrease in expression in the event of a rejection.

In some embodiments, the at least one mRNA transcript assessed in methods herein, and whose expression significantly differs between solid organ transplant recipients with rejection compared to those without rejection is of a gene involved in one or more of interferon gamma signaling, CD22-mediated BCR rejection, Rho GTPase signaling, or B cell receptor signaling. In some embodiments, such mRNA transcripts comprise transcripts of genes in one or more such pathways and also listed in Table A and/or Table 3 herein.

In some embodiments, the at least one mRNA transcript assessed in methods herein is of a gene involved in one or more biological functions such as epigenetics & transcription, autophagy, angiogenesis, MAP kinase, apoptosis & cell cycle regulation, B-cell receptor signaling, metabolism, innate immunity, lymphocyte trafficking, cytotoxicity, hematopoiesis, cytosolic DNA sensing, complement activity, adoptive immune system, MHC Class II antigen presentation, chemokine and cytokine signaling, cell-ECM interaction, inflammation, and MHC Class I antigen presentation. In some embodiments, the at least one mRNA transcript is expressed at a higher level in pancreatic acute rejection subjects, such as in T cell mediated rejection subjects, compared to in non-rejection (TX) subjects. In some embodiments the at least one mRNA transcript is of a gene listed in Table 3 below. In some embodiments, the at least one mRNA transcript has a linear fold increase in expression in acute rejection compared to no rejection of at least 2. In some embodiments, the at least one mRNA transcript is listed in Table 3 and has a linear fold increase in expression in acute rejection compared to no rejection of at least 2, as shown in Table 3.

In some embodiments, the at least one mRNA transcript comprises from 1-50 mRNA transcripts, such as from 1-10, from 1-20, or from 1-30 mRNA transcripts, such as from Table A or Table 3. In some embodiments, the at least one mRNA transcript consists of from 1-50 mRNA transcripts, such as from 1-10, from 1-20, or from 1-30 mRNA transcripts, such as from Table A or Table 3. For example, in some embodiments, the at least one mRNA transcript comprises or consists of at least 2, at least 3, at least 4, or at least 5, and up to 10, 20, 30, 50, 100, 120, or 150 transcripts listed in Table A or Table 3, such as IGHG2, IGHG1, IGHG4, IGHA1, IGLC1, IGHG3, IGKC, IRF4, IL7R, CD96, SLAMF6, SLAMF7, GZMK, IGHM, ZAP70, CD3E, CD79A, CXCR6, CD3D, MIR155HG, CTSW, SLA, IL2RG, CXCR4, ISG20, IL2RB, CCL5, PRDM1, CCR5, CCR2, AOAH, HLA-DQB1, IDO1, GZMA, IKZF1, KLRB1, TNFSF8, CD8A, IL16, HLA-DRA, CD84, BTK, NKG7, SELPLG, CD45R0, PTPRC, or ITGAX. In some embodiments, the at least one mRNA transcript comprises at least one gene from the list above, or from Table 3 or Table A. In some embodiments, the at least one mRNA transcript shows higher expression in subjects with rejection than in those with no rejection, such as at least two fold higher expression.

In some embodiments, an algorithm may be employed to determine an overall expression profile for the at least one mRNA transcript in the recipient and to compare that overall expression profile to those of exemplary expression profiles of the same mRNA transcripts in a reference sample of recipients with and without rejection. For example, in some embodiments, an algorithm may be developed that assesses such variables as the level of expression of from 2 to, for example 500, 1000, or 2000 different mRNA transcripts, and may group expression levels of mRNA transcripts of different types of genes from different biological pathways according to whether they increase or decrease with rejection, and the extent to which their levels change, and the overall importance of those pathways to the development of rejection. In some cases, a trained algorithm may be used, for example, that is adjusted and improved as more and more data from reference subjects is added to an underlying database from which the algorithm is developed. Particularly where several mRNA transcripts with different behaviors in development of rejection are used in the methods herein, an algorithm run by a computer system may be required to accurately determine whether a particular recipient's mRNA transcript expression profile indicates likelihood that the recipient has rejection or whether it indicates non-rejection. Thus, in some embodiments, a result of an algorithm is used to determine if a recipient has a gene expression profile indicating a likelihood of rejection.

In some embodiments, the expression level of the at least one mRNA transcript is determined by reverse transcription PCR (RT-PCR) (such as quantitative RT-PCR), hybridization to an array, or next generation sequencing. In some embodiments, mRNA transcript levels can be determined using a probe array. A number of distinct array formats are available. Some arrays, such as an Affymetrix HG-U133 PM microarray or other Affymetrix GeneChip® array, have different probes occupying discrete known areas of a contiguous support. Exemplary microarrays include but are not limited to the Affymetrix Human Genome U133 Plus 2.0 GeneChip or the HT HG-U133+PM Array Plate. For example, the mRNA transcripts corresponding to the genes listed in Table A may be analyzed by hybridization based on the Probe Set ID provided in Table A, on the listed HT HG-U133+PM Array (Affymetrix) provided in the Table. Alternatively, if PCR is used, appropriate PCR probes may be used that hybridize to regions near the 5′ and 3′ ends of the mRNA transcripts for the genes, such as, for example 80-120 base pairs near each end of the transcript. In some cases, nested probes or combinations of more than 2 probes may also be used to detect mRNA transcripts for particular genes. Accordingly, the expression level of the at least one mRNA transcript herein may be determined in some embodiments from a complementary DNA (cDNA) obtained from the mRNA transcript, or a double stranded DNA amplicon obtained from the mRNA transcript.

An array contains one or more probes either perfectly complementary to a particular target mRNA transcript or sufficiently complementarity to the target mRNA transcript to distinguish it from other mRNA transcripts in the sample, and the presence of such a target mRNA transcript can be determined from the hybridization signal of such probes, optionally by comparison with mismatch or other control probes included in the array. In some cases, the target bears a □ecipe□ceent label, in which case hybridization intensity can be determined by, for example, a scanning confocal microscope in photon counting mode. Appropriate scanning devices are described by e.g., U.S. Pat. Nos. 5,578,832, and 5,631,734. The intensity of labeling of probes hybridizing to a particular mRNA transcript or its amplification product provides a raw measure of expression level.

In other methods, mRNA transcript levels can be determined by so-called “real time amplification” methods also known as quantitative PCR (qPCR or qRT-PCR) or Taqman. For example, an mRNA transcript is converted to the complementary DNA sequence (cDNA) by a reverse transcriptase, and the resulting cDNA is then amplified. The basis for this method of monitoring the formation of amplification product formed during a PCR reaction with a template using oligonucleotide probes/oligos specific for a region of the template to be detected. In some embodiments, qPCR or Taqman are used immediately following a reverse-transcriptase reaction performed on isolated cellular mRNA; this variety serves to quantitate the levels of individual mRNA transcripts during qPCR.

Taqman uses a dual-labeled fluorogenic oligonucleotide probe. The dual labeled fluorogenic probe used in such assays is typically a short (ca. 20-25 bases) polynucleotide that is labeled with two different fluorescent dyes. The S′ terminus of the probe is typically attached to a reporter dye and the 3′ terminus is attached to a quenching dye. Regardless of labelling or not, the qPCR probe is designed to have at least substantial sequence complementarity with a site on the target mRNA transcript or nucleic acid derived from. Upstream and downstream PCR primers that bind to flanking regions of the locus are also added to the reaction mixture. When the probe is intact, energy transfer between the two fluorophores occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector. The recorded values can then be used to calculate the increase in normalized reporter emission intensity on a continuous basis and ultimately quantify the amount of the mRNA transcript being amplified. mRNA transcript levels can also be measured without amplification by hybridization to a probe, for example, using a branched nucleic acid probe, such as a QuantiGene® Reagent System from Panomics.

Quantitative PCR (qPCR) can also be performed without a dual-labeled fluorogenic probe by using a fluorescent dye (e.g. SYBR Green) specific for dsDNA that reflects the accumulation of dsDNA amplified specific upstream and downstream oligonucleotide primers. The increase in fluorescence during the amplification reaction is followed on a continuous basis and can be used to quantify the amount of mRNA transcript being amplified. qPCR can also be performed using microfluidics technology or digital-droplet PCR.

For qPCR or Taqman, the levels of particular genes may be expressed relative to one or more reference genes measured from the same sample using the same detection methodology. Examples include, for instance, ACTB, GAPDH, and YWHAE. Other examples include B2M, UBC, HPRT1, TTC5, C2orf44, and Chr3.

In some embodiments, for qPCR or Taqman detection, a “pre-amplification” step is performed on cDNA transcribed from cellular RNA prior to the quantitatively monitored PCR reaction. This serves to increase signal in conditions where the natural level of the RNA/cDNA to be detected is very low. Suitable methods for pre-amplification include but are not limited LM-PCR, PCR with random oligonucleotide primers (e.g. random hexamer PCR), PCR with poly-A specific primers, and any combination thereof.

In other methods, gene or nucleic acid levels can be determined by sequencing, such as by DNA sequencing. Sequencing may be performed by any available method or technique. Sequencing methods may include: Next Generation sequencing, high-throughput sequencing, pyrosequencing, classic Sanger sequencing methods, sequencing-by-ligation, sequencing by synthesis, sequencing-by-hybridization, RNA-Seq (Illumina), Digital Gene Expression (Helicos), next generation sequencing, single molecule sequencing by synthesis (SMSS) (Helicos), Ion Torrent Sequencing Machine (Life Technologies/Thermo-Fisher), massively-parallel sequencing, clonal single molecule Array (Solexa), shotgun sequencing, single molecule nanopore sequencing, sequencing by ligation, sequencing by hybridization, sequencing by nanopore current restriction, Maxim-Gilbert sequencing, primer walking, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. Conducting one or more sequencing reactions may comprise whole genome sequencing or exome sequencing.

Sequencing reactions may comprise one or more capture probes or libraries of capture probes. At least one of the one or more capture probe libraries may comprise one or more capture probes to 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250 or more genomic regions. The libraries of capture probes may be at least partially complementary. The libraries of capture probes may be fully complementary. The libraries of capture probes may be at least about 5%, 10%, 15%, 20%, %, 25%, 30%, 35%, 40%, 45%, 50%, 55%, 60%, 70%, 80%, 90%, 95%, 97% or more complementary.

Sequencing reactions may comprise one or more sets of amplification primers or libraries of amplification primers. At least one of the one or more amplification primer libraries may comprise one or more amplification primers to 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250 or more genomic regions. The libraries of amplification primers may be at least partially complementary. The libraries of amplification primers may be fully complementary. The libraries of amplification primers may be at least about 5%, 10%, 15%, 20%, %, 25%, 30%, 35%, 40%, 45%, 50%, 55%, 60%, 70%, 80%, 90%, 95%, 97% or more complementary.

In some embodiments, where the recipient is a pancreatic transplant recipient, methods used to determine level of the at least one mRNA transcript are derived from those described in U.S. Pat. No. 10,443,100 B2, which is incorporated herein by reference. In some embodiments, where the recipient is a transplant recipient, a commercial assay and algorithm such as a TruGraf® assay (Eurofins-Transplant Genomics, Framingham, MA) may be used to determine the level of the at least one mRNA transcript and whether the recipient's gene expression profile indicates likelihood of rejection on the basis of an algorithm result. In some embodiments, a result of a TruGraf® Gene Expression Profile (GEP) probability score algorithm is used. This algorithm provides results scaled on a 0-1 scale with scores >0.50 being considered positive (i.e., indicating rejection) and scores <0.50 being negative, i.e., indicating no rejection).

dd-cfDNA Determination Methods

Methods herein also involve determining dd-cfDNA in the sample, and, in particular, whether or not the level of dd-cfDNA, such as the percent dd-cfDNA out of total cfDNA in the sample, is at or above a particular pre-determined threshold indicating rejection.

There are several different methods for determining dd-cfDNA in a sample. In some methods, dd-cfDNA is determined by using genotyping data from both the donor and the recipient, for example, each obtained prior to the transplantation. In many other cases however, donor genotype data is not available. Thus, in some cases, only recipient genotype data is available and used in the method. For example, recipient genotyping may be performed on PBMC samples from the recipient. In yet other cases, neither the donor nor the recipient has been genotyped prior to determining dd-cfDNA. In some embodiments, the pre-determined threshold of dd-cfDNA is ≥0.5%, ≥0.6%, ≥0.7%, ≥0.8%, ≥0.9%, ≥1%, ≥ 1.2%, ≥1.5%, or ≥2%. In some embodiments, the dd-cfDNA threshold is ≥1.0% or >1.0%, wherein the recipient has received a pancreas transplant or both a pancreas and kidney transplant. In some cases herein, the dd-cfDNA detection methods also have a limit of detection of 0.2%, meaning that levels below 0.2% cannot be detected.

The pre-determined threshold at or above which a recipient is indicated to have a rejection may vary depending upon the amount of genotype data that is available and used in the determination. For example, donor and/or recipient genotype data is not always available for use in algorithms developed to determine the percent dd-cfDNA. Where both are available, a pre-determined threshold may be relatively low, such as ≥0.5%, ≥0.6%, ≥0.7%, ≥0.8%, ≥0.9%, ≥1% ≥1.1%, or ≥1.2% indicating rejection, as fewer assumptions are required in the method of determination. In addition, where recipient genotype data is available, in some embodiments a pre-determined threshold of ≥0.5%, ≥0.6%, ≥0.7%, ≥0.8%, ≥0.9%, ≥1%, ≥1.1%, ≥1.2%, ≥1.5%, ≥2% indicates rejection. In particular cases, a pre-determined threshold of ≥1.0% or >1.0% indicates rejection, such as when recipient genotype data are available. In other cases, the pre-determined threshold is ≥0.7% Accordingly, an “amount of dd-cfDNA” or “level of dd-cfDNA” may in some embodiments be reported as a percentage of the total cfDNA obtained from the sample.

In some embodiments, dd-cfDNA is determined by analysis of SNPs in the cfDNA obtained from the sample. For example, a donor and a recipient may have certain different SNPs at particular genetic loci, Where donor and/or recipient genotype data are available, i.e., a “two-genome” approach, particular SNP differences may be known prior to analysis. Alternatively, where genotype data for the donor and/or the recipient are not available, particular SNP differences may be found based on assaying for unique SNPs that occur in subjects with the same disease as the recipient, such as pancreatitis or pancreatic disease, with the expectation that the donor cfDNA will not show these unique SNPs. In cases where recipient and donor genotype data are not available, a higher threshold may be pre-determined for a recipient to show rejection, such as, for example, ≥1.2%, ≥1.5%, or ≥2%. In some embodiments, a particular threshold is pre-determined based on clinical studies that compare predictions of rejection based on the specific dd-cfDNA analysis algorithm used to determine the percent dd-cfDNA to actual rejection based on a surveillance biopsy result.

In a “two genomes” method that includes both recipient and donor genotype information, it may only be necessary to assay SNPs that are homozygous but differ between recipient and donor. In an approach that does not rely on donor genotype information, to quantify the observed abundance of alleles of each genotyped SNP in cfDNA sequences by sequencing, low quality reads, reads that are not mapped uniquely to the genome, and reads with potential for mapping biased by genetic variability may be filtered. Duplicated reads are then removed and allele appearances of each genotyped SNP counted (e.g. by a SAMtools mpileup function). The observed allele appearances in cfDNA and the recipient genotype are the inputs for a “one-genome” model.

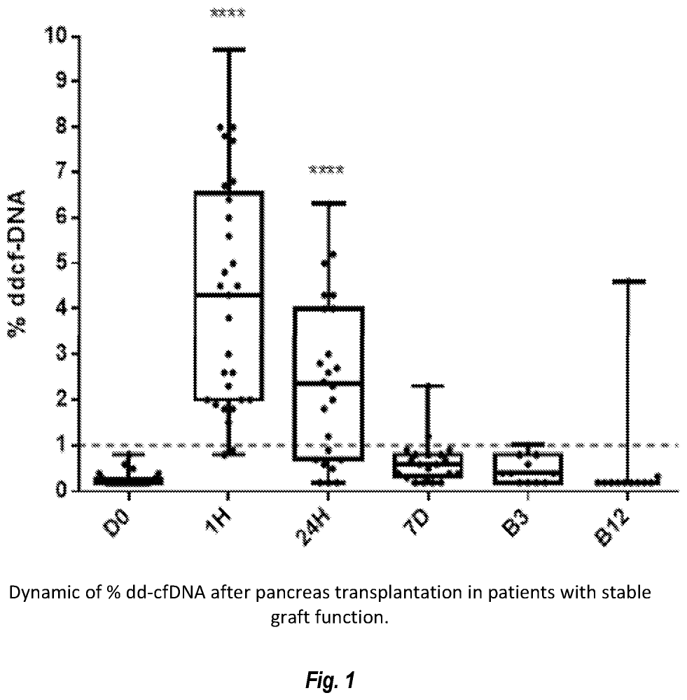

In such a one-genome model, to calculate the probability of the observed cfDNA, the probability of each possible donor and recipient genotype are first calculated. Recipient genotype can depend on the recipient measured genotype and the genotyping error rate. Since vital organ transplants are rarely closely related, the model can assume that the donor genotype is randomly selected from a human population. Given this assumption, the probability of a specific donor allele is its frequency in the population. The algorithm, in some embodiments, then iterates over the 1000 Genomes Project populations and super-populations (available from the International Genome Sample Resource (IGSR)) to detect the most likely ancestral population of the donor. To clarify further, the probability of observing a specific allele in a cfDNA fragment is computed by integrating over all possible recipient and donor genotypes and depends on the sequencing error rate, the fraction of dd-cfDNA in the □ecipeent plasma and the probabilities of observing the allele conditioning on it being donor- or recipient-derived (FIG. 1A indicated (a). Finally, the log-likelihood of the data is computed by summing log-likelihoods over all SNPs, assuming SNPs are independent (this assumption is also made by the two-genomes method). An optimization algorithm is then used to find the maximum likelihood parameter values.

In some instances, this procedure can be executed in a parallelized fashion, dramatically speeding up the determination of dd-cfNA in multiple samples or sequencing reactions (e.g. from the same individual or from multiple individuals).

An example of a “one-genome” dd-cfDNA determination without donor genotype information and an associated algorithm for determining the % dd-cfDNA is described, for example, in US Patent Application Publication No. US2021/0115506 A1 and WO2018187226A1, which are each incorporated in their entirety by reference herein. Certain commercial dd-cfDNA assays, such as a Viracor TRAC® assay (Eurofins-Viracor, Lenexa, KS, USA), Allonext assay (Eurofins Genoma), AlloSure® assay (CareDx), Prospera® assay (Natera), or TheraSure® assay (Oncocyte), may also be used in some embodiments.

In some embodiments herein, methods comprise determining the level of donor derived cell-free DNA (dd-cfDNA) in the cfDNA and the expression level of the at least one mRNA transcript in the recipient's sample, and distinguishing rejection from non-rejection in the recipient based upon results from an algorithm that considers both the dd-cfDNA and the expression level of at least one mRNA transcript and that provides a result indicating rejection or non-rejection. Thus, for example, a trained algorithm may be used that accounts for both the dd-cfDNA level and the mRNA transcript expression data collectively (i.e., in one algorithm generating one score result) rather than separately,

### Trained Algorithms

In some embodiments, methods include using a trained algorithm to analyze sample data, particularly to detect or rule-out rejection. In some embodiments, methods comprise applying a trained algorithm to the expression level of the at least one mRNA transcript and determining a result of the algorithm, wherein the result indicates rejection or non-rejection. In some embodiments, the level of dd-cfDNA is determined using a trained algorithm. A “trained algorithm” or “training algorithm,” as used herein, is an algorithm that is developed based on a set of training data, such as mRNA transcript expression levels of particular genes in subjects with or without rejection, such as tens or hundreds of such genes, or such as SNP information for SNPs throughout a genome that may differ between a donor and recipient, and developed to use the data to distinguish data profiles associated with different outcomes or phenotypes, such as rejection and non-rejection.

In such supervised learning approaches, a group of samples from two or more groups (e.g. rejection and non-rejection, as well as types of rejection such as acute cellular rejection and antibody mediated rejection) are analyzed with a statistical classification method. Differential gene or nucleic acid level data can be discovered that can be used to build a classifier that differentiates between the two or more groups, such as rejection and non-rejection. A new sample can then be analyzed so that the classifier can associate the new sample with one of the two or more groups. Examples of trained algorithms include without limitation a neural network (multi-layer perceptron), support vector machine, k-nearest neighbors, Gaussian mixture model, Gaussian, I Bayes, decision tree and radial basis function (RBF). Linear classification methods include Fisher's linear discriminant, LDA, logistic regression, I Bayes classifier, perceptron, and support vector machines (SVMs). Other algorithm methods compatible with the invention include quadratic classifiers, k-nearest neighbor, boosting, decision trees, random forests, neural networks, pattern recognition, Elastic Net, Golub Classifier, Parzen-window, Iterative RELIEF, Classification Tree, Maximum Likelihood Classifier, Nearest Centroid, Prediction Analysis of Microarrays (PAM), Fuzzy C-Means Clustering, Bayesian networks and Hidden Markov models.

Classification by a trained algorithm using supervised methods is performed in some embodiments by the following methodology:

In order to solve a given problem of supervised learning, one can consider various steps:
1. Gather a training set. These can include, for example, samples that are from recipients with known rejection and with known non-rejection, and in some cases also normal subjects, and/or subjects with particular types of rejection such as acute cellular rejection and antibody mediated rejection. These training samples are used to “train” the classifier.
2. Determine the input “feature” representation of the learned function. The accuracy of the learned function depends on how the input object is represented. Typically, the input object is transformed into a feature vector, which contains a number of features that are descriptive of the object. The number of features should not be too large, because of the curse of dimensionality; but should be large enough to accurately predict the output.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g. classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of I samples that is separate from the training set.

Once the classifier (e.g. classification model) is determined as described above, it can be used to classify a sample, e.g., that of a solid organ transplant recipient analyzed by the methods of the invention, or expression levels of particular mRNA transcripts from such a recipient.

Training of multi-dimensional algorithms may be performed using numerous samples. For example, training may be performed using at least about 10, 20, 30, 40, 50, 60, 70, 80, 90, 100, 110, 120, 130, 140, 150, 160, 170, 180, 190, 200 or more samples from subjects with known rejection or non-rejection outcomes. In some cases, training of the multi-dimensional algorithms may be performed using at least about 200, 210, 220, 230, 240, 250, 260, 270, 280, 290, 300, 350, 400, 450, 500 or more samples. In some cases, training may be performed using at least about 525, 550, 600, 650, 700, 750, 800, 850, 900, 950, 1000, 1100, 1200, 1300, 1400, 1500, 1600, 1700, 1800, 2000 or more samples.

For example, in some embodiments a trained algorithm for analyzing mRNA transcript expression data for, for example, tens or hundreds of different mRNA transcripts can be developed from a training data set of gene expression information from, for instance, several hundred transplant recipient subject samples with known rejection or non-rejection phenotypes. A Random Forest model may be trained on the dataset of the mRNA transcript levels from each subject of the dataset to generate a phenotypic classification/interpretation that predicts rejection or non-rejection in the training samples. That model may then be applied to a new sample of mRNA transcript data from a recipient whose rejection or non-rejection is unknown, providing a result indicating rejection or non-rejection for that recipient.

As trained algorithms require manipulation of many parameters simultaneously, often tens or hundreds of parameters, tracking SNPs or mRNA transcript levels, for example, they are developed and calculated using appropriate software programming methods, and may be implemented on a computer. A further discussion of computer and software implements that may be used to compute or develop a trained algorithm is provided further below.

### Distinguishing Likelihood of Different Types of Subclinical Rejection

As described in the Examples below, the present inventors discovered that the dd-cfDNA and mRNA transcript expression based assays for assessing likelihood of rejection are not redundant and, in fact, tend to correlate with different types of subclinical rejection. Specifically, the gene expression profile from analysis of mRNA transcripts preferentially detects acute cellular rejection while the dd-cfDNA assay preferentially detects antibody mediated rejection. Thus, for example, recipients with a positive result, indicating rejection, in one but not both of the assays may be more likely to have the type of rejection associated with that assay (e.g., acute cellular rejection or antibody-mediated rejection). Furthermore, use of the combined assay methods disclosed herein may assist in identifying subjects with early acute cellular rejection, which may precede a later antibody mediated rejection, allowing for therapeutic intervention that might help to reduce or inhibit an antibody mediated rejection. Accordingly, in some embodiments, the methods herein are capable of further distinguishing likelihood of acute cellular rejection from antibody-mediated rejection, wherein the dd-cfDNA level indicates presence or absence of antibody-mediated rejection, and wherein the level of the at least one mRNA transcript indicates presence or absence of acute cellular rejection.

### Methods of Treating Transplant Recipients

In some instances, the methods described herein provide information to a medical practitioner that can be useful in making a therapeutic decision. Therapeutic decisions may include decisions to: continue with a particular therapy, modify a particular therapy, alter the dosage of a particular therapy, stop or terminate a particular therapy, altering the frequency of a therapy, introduce a new therapy, introduce a new therapy to be used in combination with a current therapy, or any combination of the above. Furthermore, the methods used in this disclosure may guide the decision points in treatment regimens (e.g. addition of agents to the immunosuppression regimen due to increased evaluation of risk). For example, they may allow the evaluation of a patient with low time-of-transplant risk factors (e.g. high HLA matching between recipient and donor organ), or as having rejection, or non-rejection, justifying the adjustment of a treatment regimen.

In particular embodiments, methods herein may be used to determine whether a recipient of a solid organ transplant should or should not receive a biopsy, such as a for cause or surveillance biopsy. For example, in some embodiments, a recipient with a non-rejection result according to the methods herein, such as a negative result in each of the dd-cfDNA and mRNA transcript expression analyses or in both the dd-cfDNA and mRNA transcript expression analyses, may be determined to not be in need of a biopsy, such as a surveillance or for cause biopsy, whereas a recipient who is positive in one or both analyses may be determined to need such a biopsy. In some cases, a recipient may also receive other biomarker assays, such as an amylase and/or lipase assay or a glucose, HbAlC, or C-Peptide assay. For example, in some cases, a recipient may have received a result from an amylase, lipase, glucose, HbAlC, or C-Peptide assay indicating presence of rejection, such as a lipase level <3 ns/normal. In some cases, a method as described herein may be performed in order to further characterize the recipient and determine whether rejection is present and/or whether a for cause biopsy should be performed. Thus, in some cases, the recipient has an amylase, lipase, glucose, HbA1C, or C-Peptide assay result indicating rejection, such as a lipase result <3 ns/normal. In some cases, methods described herein are performed on such subjects prior to a biopsy, or as an alternative to a biopsy. For instance, if the instant results indicate rejection, a biopsy might not be performed but treatment may be adjusted to increase immunosuppressive treatment. In other cases, a biopsy may be performed as confirmation of the various biomarker results.

Thus, methods herein also include methods of treating a solid organ transplant recipient, wherein the recipient is determined to have a likelihood of rejection according to a method of distinguishing rejection from non-rejection herein. In some embodiments, the recipient is receiving at least one immunosuppressive drug. In some embodiments, the treatment method comprises increasing the frequency or dosage of the at least one immunosuppressant drug, administering a further immunosuppressant drug, or administering a different immunosuppressive drug to the recipient if the recipient has a positive result (indicating rejection) in the method of distinguishing rejection from non-rejection. For example, if results indicate non-rejection, an immunosuppressive drug dosage may be reduced by, for example, at least 30%, such as at least 50%, or its frequency of administration may be reduced, or it may be replaced by a weaker immunosuppressant. In some cases, if the recipient's test results indicate rejection on either or both of the dd-cfDNA and mRNA transcript expression analyses, the method of treatment comprises performing a biopsy. For example, in the case of a multi-organ transplant such as pancreas and kidney, a pancreas biopsy and/or kidney biopsy may help to determine which organ is the source of the rejection. In some cases, the dd-cfDNA and/or the mRNA transcript expression analyses are performed prior to a surveillance biopsy and such a biopsy is not ordered for the recipient unless one or both tests provide a positive result. In some cases, the recipient does not show clinical signs of rejection at the time that the recipient's sample is obtained for the dd-cfDNA and mRNA transcript expression analyses to be performed.

Many different drugs are available for treating solid organ transplant rejection, such as immunosuppressive drugs used to treat transplant rejection, such as calcineurin inhibitors (e.g., cyclosporine, tacrolimus), mTOR inhibitors (e.g., sirolimus and everolimus), anti-proliferatives (e.g., azathioprine, mycophenolic acid, mycophenolate mofetil or MMF), corticosteroids (e.g., prednisone, prednisolone, and hydrocortisone), antibodies (e.g., rituximab, basiliximab, daclizumab, muromonab-CD3, alemtuzumab, anti-thymocyte globulin and anti-lymphocyte globulin), intravenous or subcutaneous immunoglobulins (IVIG), rabbit antithymocyte globulin (rATG), interleukin 2 (IL2) receptor antagonists (e.g. basiliximab or daclizumab), and biologics (e.g. belatacept), or combinations of one or more of the above. In some embodiments, a recipient may be receiving a standard of care treatment post-transplant.

An additional immunosuppressant regimen to note is a “breakout” regimen used for treatment of any rejection episodes that occur after organ transplant. This may be a permanent adjustment to the maintenance regimen or temporary drug therapy used to minimize damage during the acute rejection episode. The adjustment may comprise temporary or long-term addition of a corticosteroid, temporary use of lymphocyte-depleting agents, and long-term addition of antiproliferative agents (e.g. mycophenolate mofetil/MMF or azathioprine, for patients not already receiving it), and any combination thereof. Treatment may also comprise plasma exchange, intravenous immunoglobulin, and anti-CD-20 antibody therapy, and any combination thereof.

In some embodiments, a method of treatment herein, if the recipient is negative in one of both of the dd-cfDNA and mRNA transcript expression analyses, indicating no rejection, comprises monitoring the recipient, including re-performing the tests at regular intervals, such as 1 week, 2 weeks, 3 weeks, 4 weeks, 2 months, 3 months, 4 months, 5 months, or 6 months, as part of a plan of active surveillance. “Active surveillance” herein refers to a treatment plan comprising regular physician visits, and optionally, regular diagnostic testing, to monitor a recipient for signs of rejection and/or organ dysfunction over a period of time. In some cases, the subject may be receiving immunosuppressive therapy, while in other cases the recipient may not be receiving therapeutics. For example, if rejection is not detected according to the dd-cfDNA and/or the mRNA transcript expression methods herein, suitable active surveillance methods of treatment may include refraining from biopsy procedures or immunosuppressant regimen adjustments for a specific period of time, such as e.g. 1 week, 2 weeks, 3 weeks, 4 weeks, 2 months, 3 months, 4 months, 5 months, or 6 months. In some cases, where a recipient is receiving immunosuppressive therapy and the methods herein indicate non-rejection, the current immunosuppressive therapy may be maintained, or may be reduced, such as through administration of a lower dose of the current drugs or by an alteration in the drugs being administered. In some cases, when rejection is not detected and the patient has previously received an increase in dose of a particular immunosuppressant of their regimen, the current increase in dose or new immunosuppressant administration may be maintained or reduced.

In some methods, expression levels and/or dd-cfDNA levels are determined at intervals in a particular patient (i.e., monitoring). Preferably, the monitoring is conducted by serial minimally-invasive tests such as blood draws; but, in some cases, the monitoring may also involve analyzing a pancreatic biopsy, either histologically or by analyzing a molecular profile. The monitoring may occur at different intervals, for example the monitoring may be hourly, daily, weekly, monthly, yearly, or some other time period, such as twice a month, three times a month, every two months, every three months, every 4 months, every 5 months, every 6 months, every 7 months, every 8 months, every 9 months, every 10 months, every 11 months, or every 12 months.

For example, if methods herein are conducted on a regular basis, they can provide an indication whether an existing immunosuppressive regimen is working, whether the immunosuppressive regimen should be changed (e.g. via administration of a new immunosuppressant to the transplant recipient or increase in dose of an immunosuppressant currently being administered to the transplant recipient) or whether a biopsy or increased monitoring by other rejection markers such as amylase, lipase, glucose, HbAlC, C-Peptide should be performed. In some cases, consecutive (e.g. at least two) tests positive for rejection as described herein indicate that an additional action be taken, e.g. adjustment of the immunosuppressive regimen (e.g. via administration of a new immunosuppressant to the transplant recipient or increase in dose of an immunosuppressant currently being administered to the transplant recipient), collection and evaluation of a biopsy, further biomarker testing. In some cases, consecutive (e.g. at least two, three, four, five, six, seven, eight, nine, ten) tests ambiguous for rejection vs. non-rejection as described herein may indicate that an additional confirmatory action be taken, e.g. collection and evaluation of a biopsy or further biomarker testing. The consecutive (e.g. at least two, three, four, five, six, seven, eight, nine, ten) tests may be separated by an appropriate time period (e.g. one day, one week, two weeks, three weeks, one month, two months, three months, four months, five months, six months, or one year) to ensure that the tests accurately represent a trend.

Treatment methods provided herein include administering a blood test (e.g., a test to detect subclinical acute rejection) to a transplant recipient who has already undergone a surveillance biopsy of the pancreas and received a biopsy result in the form of a histological analysis or a molecular profiling analysis. In some particular instances, the analysis of the biopsy (e.g., by histology or molecular profiling) may result in ambiguous, inconclusive or borderline results. In such cases, a blood test provided herein may assist a caregiver with determining whether the transplant recipient has subclinical acute rejection or with interpreting the biopsy. In other cases, the biopsy itself may be inconclusive or ambiguous, and in such cases the molecular analysis of the biopsy may be used in adjunct with the histology to confirm a diagnosis. In some instances, the analysis of the biopsy may yield a negative result. In such cases, the subject may receive a dd-cfDNA and/or a mRNA transcript expression analysis as provided herein in order to confirm the negative result, or to detect subclinical acute rejection. In some cases, after receiving any type of biopsy result (e.g., negative result, ambiguous, inconclusive, borderline, positive), the patient may receive multiple, serial dd-cfDNA and/or mRNA transcript expression analyses as described herein, in order to monitor changes in molecular markers correlated with subclinical acute rejection.

Treatment methods provided herein also include performing a biopsy on a transplant recipient who has received a dd-cfDNA and/or mRNA transcript expression analysis as described herein. In some embodiments, the recipient is positive for both the dd-cfDNA and mRNA transcript expression analysis portions of the methods, indicating rejection. In other cases, the recipient is positive only for dd-cfDNA or for mRNA transcript expression results. In cases where only the dd-cfDNA or mRNA transcript expression conducted independently on a sample yields a positive result, or where such result is borderline (i.e., at or near the threshold separating rejection from non-rejection), the patient's healthcare worker may use the results of a biopsy test as a complement in order to confirm whether rejection is present. As noted above, a biopsy of the organs of a multi-organ transplant such as a pancreas and kidney transplant (PAK or SPK) may also be used in cases indicating rejection to determine whether the rejection is in one organ only or in all organs.

### Computer Implemented Methods and Systems for Conducting Methods Herein

As described previously, gene or nucleic acid levels can be analyzed and associated with status of a subject (e.g., presence or absence of rejection) in a digital computer, while algorithms herein, such as trained algorithms may be applied through use of a computer. In some embodiments, a sample is first collected from a subject (for example, from a transplant recipient). The sample is assayed and nucleic acid products are generated. A computer system is used In analyzing the data and making a classification of rejection or non-rejection based on, for example, results from the dd-cfDNA and/or the expression level of at least one mRNA transcript or both, wherein rejection in the recipient is indicated by either or both of (i) a level of dd-cfDNA at or above a pre-determined threshold value, and (ii) expression level of the at least one mRNA transcript or a result of an algorithm based on the expression level indicating rejection, or alternatively, wherein rejection in the recipient is indicated by result of an algorithm accounting for both the dd-cfDNA level and the mRNA transcript expression level data.

In some embodiments of the disclosure, a system that is capable of determining the level of each of dd-cfDNA and then the expression of the at least one mRNA transcript on a separate sample is used to conduct methods herein. In some cases, such a system may include components for conducting assays to determine the level of one or both of dd-cfDNA level and expression level of the at least one mRNA transcript. In some cases, alternatively or additionally, a system may include a computer and appropriate software for conducting one or more algorithms, such as trained algorithms, in order to determine the level of dd-cfDNA and expression of at least one mRNA transcript from a recipient sample. A system may comprise software that provides an algorithm result for a recipient, for example, positive or negative (i.e. rejection or no rejection), for each of the dd-cfDNA and/or mRNA transcript expression analyses, or for both analyses in combination, which may then in some embodiments be provided to a caregiver for the recipient in order to determine further treatment steps for the recipient.

Optionally, in a system herein, a computer is directly linked to a scanner or the like receiving experimentally determined signals related to gene or nucleic acid levels, (i.e., for SNP identification or identification of the levels of various expressed mRNA transcripts), and the like. Alternatively, gene or nucleic acid levels can be input by other means. The computer can be programmed to convert raw signals into gene or nucleic acid levels (absolute or relative), compare measured gene or nucleic acid levels with one or more reference levels, or a scale of such values, as described above. The computer can also be programmed to assign values or other designations to gene or nucleic acid levels based on the comparison with one or more reference gene or nucleic acid levels, and to aggregate such values or designations for multiple gene or nucleic acids in a profile. The computer can also be programmed to output a value or other designation providing an indication of rejection or non-rejection as well as any of the raw or intermediate data used in determining such a value or designation.

The methods provided herein may also be capable of generating and transmitting results through a computer network. A sample is first collected from a subject (e.g. transplant recipient). The sample is assayed and gene or nucleic acid levels are generated. A computer system is used in analyzing the data and making classification of the sample. The result is capable of being transmitted to different types of end users via a computer network. In some instances, the subject (e.g. patient) may be able to access the result by using a standalone software and/or a web-based application on a local computer capable of accessing the internet. In some instances, the result can be accessed via a mobile application provided to a mobile digital processing device (e.g. mobile phone, tablet, etc.). In some instances, the result may be accessed by physicians and help them identify and track conditions of their patients. In some instances, the result may be used for other purposes such as education and research.

The methods disclosed herein may include at least one computer program, or use of the same. A computer program may include a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. The computer program will normally provide a sequence of instructions from one location or a plurality of locations. In various embodiments, a computer program includes, in part or in whole, one or more web applications, one or more mobile applications, one or more standalone applications, one or more web browser plug-ins, extensions, add-ins, or add-ons, or combinations thereof. Further disclosed herein are systems for classifying one or more samples and uses thereof. The system may comprise (a) a digital processing device comprising an operating system configured to perform executable instructions and a memory device; (b) a computer program including instructions executable by the digital processing device to classify a sample from a subject comprising: (i) a first software module configured to receive a an gene or nucleic acid level profile of one or more genes from the sample from the subject; (ii) a second software module configured to analyze the gene or nucleic acid level profile from the subject; and (iii) a third software module configured to classify the sample from the subject based on a classification system comprising two or more classes (e.g. rejection vs. non-rejection).

The system is in communication with a processing system. The processing system can be configured to implement the methods disclosed herein. In some examples, the processing system is a microarray scanner. In some examples, the processing system is a real-time PCR machine (optionally microfluidic). In some examples, the processing system is a nucleic acid sequencing system, such as, for example, a next generation sequencing system (e.g., Illumina sequencer, Ion Torrent sequencer, Pacific Biosciences sequencer, BGI sequencing system). The processing system can be in communication with the system through the network, or by direct (e.g., wired, wireless) connection. The processing system can be configured for analysis, such as nucleic acid sequence analysis.

Methods as described herein can be implemented by way of machine (or computer processor) executable code (or software) stored on an electronic storage location of the system, such as, for example, on the memory or electronic storage unit. During use, the code can be executed by the processor. In some examples, the code can be retrieved from the storage unit and stored on the memory for ready access by the processor. In some situations, the electronic storage unit can be precluded, and machine-executable instructions are stored on memory.

### Digital Processing Device

Systems herein for conducting the methods may include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles.

The digital processing device will normally include an operating system configured to perform executable instructions. The operating system is, for example, software, including programs and data, which manages the device's hardware and provides services for execution of applications. Exemplary operating systems include, by way of non-limiting examples, FreeBSD, OpenBSD, NetBSD®, Linux, Apple® Mac OS X Server®, Oracle® Solaris®, Windows Server®, and Novell® NetWare “, as well as the personal computer operating systems such as Microsoft® Windows®, Apple® Mac OS X”, UNIX®, and UNIX-like operating systems such as GNU/Linux®. In some embodiments, the operating system is provided by cloud computing. Mobile smart phone operating systems include, by way of non-limiting examples, Nokia® Symbian® OS, Apple® iOS®, Research In Motion® BlackBerry OS®, Google® Android®, Microsoft® Windows Phone& OS, Microsoft® Windows Mobile® OS, Linux®, and Palm® WebOS®.

The digital processing device generally includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, or other external memory devices.

A digital processing device may also include a display to send visual information to a user. The digital processing device may include an input device to receive information from a user, e.g., from a keyboard or touch screen or other means of inputting information.

### Computer Programs

The systems disclosed herein may include one or more non-transitory computer readable storage media encoded with a program including instructions executable by the operating system to perform and analyze the test described herein; preferably connected to a networked digital processing device. A non-transitory computer-readable storage media may be encoded with a computer program including instructions executable by a processor to create or use an algorithm to determine one or more results for methods herein (i.e. levels of dd-cfDNA or parameters needed to determine level of dd-cfDNA, and/or levels of particular mRNA transcripts, or expression profiles from the at least one mRNA transcript). The storage media may comprise a database, in a computer memory, of one or more clinical features of control samples, for example, or of other data or parameters used in algorithms of the methods, or in creating a trained algorithm.

In some embodiments, a computer program includes a web application. In light of the disclosure provided herein, those of skill in the art will recognize that a web application, in various embodiments, utilizes one or more software frameworks such as Microsoft® NET or Ruby on Rails (RoR), and one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

In some embodiments, the computer program includes a web browser plug-in. In computing, a plug-in is one or more software components that add specific functionality to a larger software application. Examples of web browser plug-ins include Adobe® Flash® Player, Microsoft® Silverlight®, and Apple® QuickTime®.

Software modules connected with methods herein may be created by techniques known to those of skill in the art using machines, software, and languages known to the art.

### Databases

The systems disclosed herein may comprise one or more databases, or use of the same. Exemplary databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Data Transmission and Reports

Methods herein may further comprise providing one or more reports, and systems herein for conducting such methods may include means for generating such reports. The one or more reports may comprise a status or outcome of a transplant in a subject, i.e. whether the method indicates rejection or non-rejection. The one or more reports may also comprise information pertaining to therapeutic regimens for use in treating transplant rejection or in suppressing an immune response in a recipient, such as based on the results of the method. The one or more reports may be transmitted to a recipient or to a medical representative of the recipient such as a physician, physician's assistant, nurse, or other medical personnel, or to a family member, guardian, or legal representative of the subject.

### EXAMPLES

### Example 1. Improved Methods of Detection of Pancreatic Transplant Rejection by Combining Blood Gene Expression and Cell Free DNA Analysis

Donor-derived cell-free DNA (dd-cfDNA) is a noninvasive test that had demonstrated high predictive performance for acute rejection in solid organ transplant. However, combining dd-cfDNA with a gene expression profile assay could improve the diagnostic of subclinical acute rejection episodes. We aimed at evaluating the combination of these two molecular diagnostic methods in pancreas transplantation.

We conducted a prospective longitudinal study including all pancreas transplant recipients from January 2017 to December 2019. Plasma samples were collected before transplant (DO), and at 1 h, 24 h and 7 days (D7) post-transplant, and at time of pancreas biopsy-either surveillance at 3 weeks (B3) and 12 months (B12), or per clinical indication. Biopsies were classified according to the Banff criteria.

Dd-cfDNA percentage (%) was assessed using Eurofins Genoma AlloNext® by Next Generation Sequencing (NGS). This assessment presents a positive predictive value of 86% and a specificity of 93% for the diagnosis of acute rejection. See also Example 2 under the heading “donor-derived cell-free DNA analysis” for further details.

Blood samples for the gene expression profile assay were drawn and obtained. The samples were processed using Affymetrix HT-HG-U133+PM Array Plates on the Gene Titan MC instrument (Thermo Fisher Scientific, Waltham, MA) (deposited as GEO Accession No. GSE107509) according to manufacturer's instructions. The samples were further processed as described in Example 2 under the heading: “gene expression provile analysis.” In this specific example, the gene expression profiles were analyzed with the TruGraf® algorithm-a DNA microarray-based and/or qPCR-based gene expression algorithm analyzing differential expression of the genes listed in Table A and assigned a result of either TX or not-TX. Gene expression profile results were provided as a probability score normalized on a 0-100 scale. The TruGraf® assay (Eurofins-Transplant Genomics, Framingham, MA) has a previously defined probability threshold of 50 to differentiate the TX (normal, no rejection) from the not-TX phenotype (including subclinical rejection). The genes used for the TruGraf® assay are described in Table A below. The human genes listed in Table A are identified by their full name and gene symbols, as well as by the Probe Set ID provided for each of the genes in the Affymetrix HG-U133 Plus PM microarray (Array Name “HT_HG-U133_Plus_PM”) and their Ensemble ID. Also included are the gene title abbreviation, full name of the gene, and alternative gene title abbreviations. This TruGraf® assay presents a negative predictive value of 86% and a specificity of 83% for diagnosis of acute rejection.

TABLE A
Probes and corresponding genes assessed in the TruGraf ® gene expression assay.

| | | | Gene | | |
|---|---|---|---|---|---|
| # | Probe Set Id | ENSEMBL ID | Symbol | Gene Title | Alternate names |
| 1 | 1561058_PM_at | ENSG00000151702 | FLI1 | Fli-1 proto-oncogene, ETS transcription factor | EWSR2, FLI-1, SIC-1 |
| 2 | 1562505_PM_at | | LOC255177 | Hs03671216_m1 uncharacterized LOC255177 | |
| 3 | 1565913_PM_at | ENSG00000105939 | ZC3HAV1 | zinc finger CCCH-type containing, antiviral 1 | ARTD13, FLB6421, FLJ13288, |
| | | | | | MGC48898, PARP13, ZAP, |
| | | | | | ZC3H2, ZC3HDC2 |
| 4 | 1570264_PM_at | ENSG00000075568 | TMEM131 | transmembrane protein 131 | CC28, KIAA0257, PRO1048, |
| | | | | | RW1, YR-23 |
| 5 | 232375_PM_at | ENSG00000006062 | MAP3K14 | mitogen-activated protein kinase 14 | FTDCR1B, HS, HSNIK, NIK |
| 6 | 232405_PM_at | ENSG00000150961 | SEC24D | SEC24 homolog D, COPII coat complex | CLCRP2, KIAA0755 |
| | | | | component | |
| 7 | 233309_PM_at | ENSG00000198754 | OXCT2 | 3-oxoacid CoA-transferase 2 | FKSG25, SCOTT |
| 8 | 240008_PM_at | ENSG00000226091 | LINC00937 | long intergenic non-protein coding RNA 937 | |
| 9 | 242014_PM_at | ENSG00000121988 | ZRANB3 | zinc finger RANBP2-type containing 3 | 4933425L19Rik, AH2 |
| 10 | 242374_PM_at | ENSG00000156875 | MFSD14A | major facilitator superfamily domain containing | HIAT1 |
| | | | | 14A | |
| 11 | 242751_PM_at | ENSG00000117592 | PRDX6 | peroxiredoxin 6 | 1-Cys, AOP2, HEL-S-128m, |
| | | | | | NSGPx, PRX, aiPLA2, p29 |
| 12 | 244433_PM_at | ENSG00000179598 | PLD6 | phospholipase D family member 6 | ZUC |
| 13 | 222064_PM_s_at | ENSG00000266967 | AARSD1 | alanyl-tRNA synthetase domain containing 1 | ALAXP, MGC2744 |
| 14 | 202366_PM_at | ENSG00000122971 | ACADS | acyl-CoA dehydrogenase, C-2 to C-3 short chain | ACAD3, SCAD |
| 15 | 211207_PM_s_at | ENSG00000164398 | ACSL6 | acyl-CoA synthetase long-chain family member 6 | ACS2, FACL6, KIAA0837, |
| | | | | | LACS2, LACS5 |
| 16 | 200801_PM_x_at | ENSG00000075624 | ACTB | actin beta | |
| 17 | 232864_PM_s_at | ENSG00000072364 | AFF4 | AF4/FMR2 family, member 4 | AF5Q31, MCEF |
| 18 | 225108_PM_at | ENSG00000018510 | AGPS | alkylglycerone phosphate synthase | ADAP-S, ADAS, ADHAPS, |
| | | | | | ADPS, ALDHPSY |
| 19 | 201612_PM_at | ENSG00000143149 | ALDH9A1 | aldehyde dehydrogenase 9 family, member A1 | ALDH4, ALDH7, ALDH9, E3 |
| 20 | 200613_PM_at | ENSG00000161203 | AP2M1 | adaptor related protein complex 2 subunit mu 1 | AP50, CLAPM1, MU2 |
| 21 | 221013_PM_s_at | ENSG00000128335 | APOL2 | apolipoprotein L2 | APOL-II |
| 22 | 1557116_PM_at | ENSG00000221963 | APOL6 | apolipoprotein L6 | APOL-VI, APOLVI |
| 23 | 218527_PM_at | ENSG00000137074 | APTX | aprataxin | AOA, AOA1, AXA1, EAOH, |
| | | | | | EOAHA, FLJ20157 |
| 24 | 1555812_PM_a_at | ENSG00000111348 | ARHGDIB | Rho GDP dissociation inhibitor (GDI) beta | GDIA2, GDID4, LY-GDI, |
| | | | | | RAP1GN1, RHOGDI2 |
| 25 | 222524_PM_s_at | ENSG00000148331 | ASB6 | ankyrin repeat and SOCS box containing 6 | |
| 26 | 200041_PM_s_at | ENSG00000198563 | DDX39B | DexD-box helicase 39B | BAT1, D6S81E, UAP56 |
| 27 | 204516_PM_at | ENSG00000163635 | ATXN7 | ataxin 7 | ADCAII, OPCA3, SCA7, |
| | | | | | SGF73 |
| 28 | 210125_PM_s_at | ENSG00000175334 | BANF1 | barrier to autointegration factor 1 | BAF |
| 29 | 219966_PM_x_at | ENSG00000172530 | BANP | BTG3 associated nuclear protein | BEND1, DKFZP761H172, |
| | 228928_PM_x_at | | | | FLJ10177, FLJ20538, SMAR1, |
| | 233186_PM_s_at | | | | SMARBP1 |
| 30 | 222891_PM_s_at | ENSG00000119866 | BCL11A | BCL11 transcription factor A | BCL11A-L, BCL11A-S, |
| | | | | | BCL11A-XL, CTIP1, EVI9, |
| | | | | | HBFQTL5, SMARCM1, |
| | | | | | ZNF856 |
| 31 | 219191_PM_s_at | ENSG00000110934 | BIN2 | bridging integrator 2 | BRAP-1 |
| 32 | 59644_PM_at | ENSG00000138756 | BMP2K | BMP2 inducible kinase | BIKE, DKFZP434K0614 |
| 33 | 204167_PM_at | ENSG00000169814 | BTD | biotinidase | |
| 34 | 201236_PM_s_at | ENSG00000159388 | BTG2 | BTG anti-proliferation factor 2 | APRO1, MGC126063, |
| | | | | | MGC126064, PC3, TIS21 |
| 35 | 50314_PM_i_at | ENSG00000101220 | C20orf27 | chromosome 20 open reading frame 27 | ADISSP, FLJ20550 |
| 36 | 200623_PM_s_at | ENSG00000143933 | CALM2 | calmodulin 2 (phosphorylase kinase, delta) | CAMII, PHKD, PHKD2 |
| 37 | 200623_PM_s_at | ENSG00000160014 | CALM3 | calmodulin 3 (phosphorylase kinase, delta) | PHKD, PHKD3 |
| 38 | 225933_PM_at | ENSG00000185298 | CCDC137 | coiled-coil domain containing 137 | MGC16597 |
| 39 | 227458_PM_at | ENSG00000120217 | CD274 | CD274 molecule | B7-H, B7-H1, B7H1, PD-L1, |
| | | | | | PDCD1LG1, PDL1 |
| 40 | 205297_PM_s_at | ENSG00000007312 | CD79B | CD79b molecule, immunoglobulin- associated | B29, IG-BETA, IGB, IGBETA |
| | | | | beta | |
| 41 | 214907_PM_at | ENSG00000007129 | CEACAM21 | carcinoembryonic antigen-related cell adhesion | FLJ13540, R29124_1 |
| | | | | molecule 21 | |
| 42 | 1555730_PM_a_at | ENSG00000172757 | CFL1 | cofilin 1 (non-muscle) | CFL |
| 43 | 203921_PM_at | ENSG00000175040 | CHST2 | carbohydrate (N-acetylglucosamine-6-O) | C6ST |
| | | | | sulfotransferase 2 | |
| 44 | 203536_PM_s_at | ENSG00000144021 | CIAO1 | cytosolic iron-sulfur assembly component 1 | CIA1, WDR39 |
| 45 | 223465_PM_at | ENSG00000113163 | CERT1 | ceramide transporter 1 | CERT, COL4A3BP, GPBP, |
| | | | | | STARD11 |
| 46 | 235533_PM_at | ENSG00000240230 | COX19 | cytochrome c oxidase assembly factor COX19 | MGC104475 |
| 47 | 219805_PM_at | ENSG00000018610 | STEEP1 | STING1 ER exit protein 1 | CXORF56, FLJ22965 |
| 48 | 222996_PM_s_at | ENSG00000171604 | CXXC5 | CXXC finger protein 5 | HSPC195, RINF, WID |
| | 224516_PM_s_at | | | | |
| 49 | 205603_PM_s_at | ENSG00000147202 | DIAPH2 | diaphanous-related formin 2 | DIA, DIA2, POF, POF2 |
| 50 | 1565680_PM_at | ENSG00000151240 | DIP2C | disco interacting protein 2 homolog C | KIAA0934 |
| 51 | 212467_PM_at | ENSG00000138246 | DNAJC13 | DnaJ (Hsp40) homolog, subfamily C, member 13 | KIAA0678, RME8 |
| 52 | 201537_PM_s_at | ENSG00000108861 | DUSP3 | dual specificity phosphatase 3 | VHR |
| 53 | 203729_PM_at | ENSG00000142227 | EMP3 | epithelial membrane protein 3 | YMP |
| 54 | 204143_PM_s_at | ENSG00000132199 | ENOSF1 | enolase superfamily member 1 | FUCD, HSRTSBETA, RTS, |
| | | | | | TYMSAS |
| 55 | 235645_PM_at | ENSG00000141446 | ESCO1 | establishment of sister chromatid cohesion N- | EFO1, ESO1, KIAA1911 |
| | | | | acetyltransferase 1 | |
| 56 | 224926_PM_at | ENSG00000131558 | EXOC4 | exocyst complex component 4 | KIAA1699, MGC27170, SEC8, |
| | | | | | SEC8L1, SEC8P |
| 57 | 227410_PM_at | ENSG00000185112 | FAM43A | family with sequence similarity 43, member A | FLJ90022 |
| 58 | 212229_PM_s_at | ENSG00000135108 | FBXO21 | F-box protein 21 | FBX21, KIAA0875 |
| 59 | 216950_PM_s_at | ENSG00000150337 | FCGR1A | Fc gamma receptor Ia | CD64, CD64A, FCG1, |
| | | | | | FCGAMMARI, |
| | | | | | FCGAMMARIA, FCGR1 |
| 60 | 216950_PM_s_at | ENSG00000265531 | FCGR1C | Fc gamma receptor Ic, pseudogene | CD64C, FCGR1C |
| 61 | 214511_PM_x_at | ENSG00000198019 | FCGR1B | Fc gamma receptor Ib, pseudogene | CD64B, FCGR1B |
| 62 | 210889_PM_s_at | ENSG00000072694 | FCGR2B | Fc gamma receptor Iib | CD32, CD32B, FCG2, |
| | | | | | FCGAMMARIIB, FCGR2, |
| | | | | | FCGRIIB |
| 63 | 224840_PM_at | ENSG00000096060 | FKBP5 | FK506 binding protein 5 | FKBP51, FKBP54, P54, |
| | | | | | PPIASE, PTG-10 |
| 64 | 223950_PM_s_at | ENSG00000059122 | FLYWCH1 | FLYWCH-type zinc finger 1 | DKFZP761A132 |
| 65 | 209304_PM_x_at | ENSG00000099860 | GADD45B | growth arrest and DNA-damage-inducible, beta | DKFZP566B133, |
| | | | | | GADD45BETA, MYD118 |
| 66 | 212581_PM_x_at | ENSG00000111640 | GAPDH | glyceraldehyde-3-phosphate dehydrogenase | GAPD |
| 67 | 201040_PM_at | ENSG00000114353 | GNAI2 | G protein subunit alpha i2 | GIP, GNAI2B |
| 68 | 200745_PM_s_at | ENSG00000078369 | GNB1 | guanine nucleotide binding protein (G protein), | |
| | | | | beta polypeptide 1 | |
| 69 | 205495_PM_s_at | ENSG00000115523 | GNLY | granulysin | D2S69E, LAG-2, LAG2, |
| | | | | | NKG5, TLA519 |
| 70 | 210999_PM_s_at | ENSG00000106070 | GRB10 | growth factor receptor bound protein 10 | |
| 71 | 219233_PM_s_at | ENSG00000073605 | GSDMB | gasdermin B | GSDML, PRO2521 |
| 72 | 225012_PM_at | ENSG00000115677 | HDLBP | high density lipoprotein binding protein | HBP, VGL, HS00245546_m1 |
| 73 | 200904_PM_at | ENSG00000233904 | HLA-E | major histocompatibility complex, class I, E | |
| 74 | 217436_PM_x_at | ENSG00000204622 | HLA-J | major histocompatibility complex, class I, J | CDA12, D6S203, HLA-59, |
| | | | | (pseudogene) | HLA-CDA12 |
| 75 | 212431_PM_at | ENSG00000113716 | HMGXB3 | HMG-box containing 3 | HMGX3, KIAA0194, SMF |
| 76 | 219620_PM_x_at | ENSG00000171159 | BBLN | bublin coiled coil protein | C9ORF16, EST00098, |
| | | | | | FLJ12823, MGC4639 |
| 77 | 230950_PM_at | ENSG00000133313 | CNDP2 | carnosine dipeptidase 2 | CN2, CPGL, FLJ10830, |
| | | | | | HST2298, PEPA, |
| | | | | | Hs_00924031_m1 |
| 78 | 221219_PM_s_a | ENSG00000104731 | KLHDC4 | kelch domain containing 4 | DKFZP434G0522 |
| 79 | 229562_PM_at | ENSG00000106211 | HSPB1 | heat shock protein family B (small) member 1 | CMT2F, HS.76067, HSP25, |
| | | | | | HSP27, HSP28 |
| 80 | 210253_PM_at | ENSG00000109854 | HTATIP2 | HIV-1 Tat interactive protein 2 | CC3, FLJ26963, SDR44U1, |
| | | | | | TIP30 |
| 81 | 207194_PM_s_at | ENSG00000105371 | ICAM4 | intercellular adhesion molecule 4 (Landsteiner- | CD242, LW |
| | | | | Wiener blood group) | |
| 82 | 232171_PM_x_at | ENSG00000142166 | IFNAR1 | interferon alpha and beta receptor subunit 1 | IFNAR, IFRC |
| 83 | 214669_PM_x_at | ENSG00000211592 | IGKC | immunoglobulin kappa constant | HCAK1 |
| 84 | 201234_PM_at | ENSG00000166333 | ILK | integrin linked kinase | |
| 85 | 222471_PM_s_at | ENSG00000176407 | KCMF1 | potassium channel modulatory factor 1 | DEBT91, DKFZP434L1021, |
| | | | | | PCMF, ZZZ1 |
| 86 | 226518_PM_at | ENSG00000110906 | KCTD10 | potassium channel tetramerization domain | BTBD28, MSTP028 |
| | | | | containing 10 | |
| 87 | 238001_PM_at | ENSG00000168301 | KCTD6 | potassium channel tetramerization domain | KCASH3, MGC27385 |
| | | | | containing 6 | |
| 88 | 219471_PM_at | ENSG00000102445 | RUBCNL | rubicon like autophagy enhancer | C13ORF18, FLJ21562, |
| | 44790_PM_s_at | | | | KIAA0226L, PACER |
| 89 | 208784_PM_s_at | ENSG00000124702 | KLHDC3 | kelch domain containing 3 | DJ20C7.3, HPEAS, PEAS |
| 90 | 1566129_PM_at | ENSG00000169756 | LIMS1 | LIM and senescent cell antigen-like domains 1 | PINCH, PINCH1 |
| 91 | 1556033_PM_at | ENSG00000274020 | LINC01138 | long intergenic non-protein coding RNA 1138 | FLJ39739, LINC00875 |
| 92 | 224767_PM_at | ENSG00000145592 | RPL37 | ribosomal protein L37 | L37 |
| 93 | 229187_PM_at | ENSG00000273748 | LOC283788 | FSHD region gene 1 pseudogene | |
| 94 | 225680_PM_at | ENSG00000161036 | LRWD1 | leucine rich repeats and WD repeat domain | CENP-33, DKFZP434K1815, |
| | | | | containing 1 | ORCA |
| 95 | 224559_PM_at | ENSG00000251562 | MALAT1 | metastasis associated lung adenocarcinoma | HCN, LINC00047, MALAT-1, |
| | 224549_PM_x_at | | | transcript 1 | MASCRNA, NCRNA00047, |
| | | | | | NEAT2, PRO1073 |
| 96 | 232420_PM_x_at | ENSG00000268996 | MAN1B1- | MAN1B1 divergent transcript | MAN1B1-AS1 |
| | | | DT | | |
| 97 | 218438_PM_s_at | ENSG00000118579 | MED28 | mediator complex subunit 28 | DKFZP434N185, EG1, |
| | | | | | MAGICIN |
| 98 | 227787_PM_s_at | ENSG00000164758 | MED30 | mediator complex subunit 30 | THRAP6, TRAP25 |
| 99 | 209199_PM_s_at | ENSG00000081189 | MEF2C | myocyte enhancer factor 2C | |
| 100 | 232340_PM_at | ENSG00000244625 | MIATNB | MIAT neighbor | |
| 101 | 1558120_PM_at | ENSG00000197530 | MIB2 | MIB E3 ubiquitin protein ligase 2 | FLJ39787, SKELETROPHIN, |
| | | | | | ZZANK1, ZZZ5 |
| 102 | 205905_PM_s_at | ENSG00000204520 | MICA | MHC class I polypeptide-related sequence A | PERB11.1 |
| 103 | 205905_PM_s_at | ENSG00000204516 | MICB | MHC class I polypeptide-related sequence B | PERB11.2 |
| 104 | 210356_PM_x_at | ENSG00000156738 | MS4A1 | membrane-spanning 4-domains, subfamily A, | B1, BP35, CD20, FMC7 |
| | 217418_PM_x_at | | | member 1 | |
| 105 | 225232_PM_at | ENSG00000150712 | MTMR12 | myotubularin related protein 12 | 3-PAP, 3PAP, FLJ20476, |
| | | | | | KIAA1682, PIP3AP |
| 106 | 219320_PM_at | ENSG00000278259 | MYO19 | myosin XIX | FLJ22865, MYOHD1 |
| 107 | 203072_PM_at | ENSG00000197879 | MYO1C | myosin IC | MYOIC, MYR2, NMI |
| 108 | 227937_PM_at | ENSG00000176182 | MYPOP | Myb related transcription factor, partner of | P42POP |
| | | | | profilin | |
| 109 | 242918_PM_at | ENSG00000132780 | NASP | nuclear autoantigenic sperm protein (histone- | DKFZP547F162, FLB7527, |
| | | | | binding) | FLJ31599, FLJ35510, |
| | | | | | MGC19722, MGC20372, |
| | | | | | MGC2297, PRO1999 |
| 110 | 219100_PM_at | ENSG00000107960 | STN1 | STN1 subunit of CST complex | BA541N10.2, FLJ22559, |
| | | | | | OBFC1 |
| 111 | 215080_PM_s_at | ENSG00000135506 | OS9 | OS9 endoplasmic reticulum lectin | ERLEC2, OS-9 |
| 112 | 231035_PM_s_at | ENSG00000165312 | OTUD1 | I deubiquitinase 1 | DUBA7, OTDC1 |
| 113 | 1553856_PM_s_at | ENSG00000078589 | P2RY10 | purinergic receptor P2Y, G-protein coupled, 10 | P2Y10 |
| 114 | 236298_PM_at | ENSG00000148459 | PDSS1 | prenyl (decaprenyl) diphosphate synthase, | COQ1, COQ1A, TPRT, TPT |
| | | | | subunit 1 | |
| 115 | 229219_PM_s_at | ENSG00000275498 | PDXDC1 | pyridoxal dependent decarboxylase domain | KIAA0251 |
| | | | | containing 1 | |
| 116 | 200634_PM_at | ENSG00000108518 | PFN1 | profilin 1 | |
| 117 | 239294_PM_at | ENSG00000105851 | PIK3CG | phosphatidylinositol-4,5-bisphosphate 3- kinase, | |
| | | | | catalytic subunit gamma | |
| 118 | 201251_PM_at | ENSG00000067225 | PKM | pyruvate kinase M1/2 | OIP3, PK3, PKM2, THBP1 |
| 119 | 204195_PM_s_at | ENSG00000160199 | PKNOX1 | PBX/knotted 1 homeobox 1 | PREP1 |
| 120 | 222582_PM_at | ENSG00000106617 | PRKAG2 | protein kinase, AMP-activated, gamma 2 non- | AAKG, AAKG2, CMH6, |
| | | | | catalytic subunit | H91620P, WPWS |
| 121 | 216069_PM_at | ENSG00000160310 | PRMT2 | protein arginine methyltransferase 2 | HRMT1L1, MGC111373 |
| 122 | 219938_PM_s_at | ENSG00000152229 | PSTPIP2 | proline-serine-threonine phosphatase | |
| | | | | interacting protein 2 | |
| 123 | 203111_PM_s_at | ENSG00000120899 | PTK2B | protein tyrosine kinase 2 beta | CADTK, CAKB, FAK2, PTK, |
| | | | | | PYK2, RAFTK |
| 124 | 217763_PM_s_at | ENSG00000168461 | RAB31 | RAB31, member RAS oncogene family | RAB22B |
| 125 | 221439_PM_at | ENSG00000089050 | RBBP9 | RB binding protein 9, serine hydrolase | BOG |
| 126 | 222026_PM_at | ENSG00000102317 | RBM3 | RNA binding motif protein 3 | IS1-RNPL |
| 127 | 201395_PM_at | ENSG00000003756 | RBM5 | RNA binding motif protein 5 | H37, LUCA-15, LUCA15 |
| 128 | 223989_PM_s_at | ENSG00000076043 | REXO2 | RNA exonuclease 2 | CGI-114, DKFZP566E144, SFN |
| 129 | 212099_PM_at | ENSG00000143878 | RHOB | ras homolog family member B | ARH6, ARHB, MST081, |
| | | | | | RHOH6 |
| 130 | 200885_PM_at | ENSG00000155366 | RHOC | ras homolog family member C | ARH9, ARHC |
| 131 | 225416_PM_at | ENSG00000131263 | RLIM | ring finger protein, LIM domain interacting | MGC15161, NY-REN-43, |
| | | | | | RNF12 |
| 132 | 1555916_PM_at | ENSG00000156990 | RPUSD3 | RNA pseudouridine synthase D3 | MGC29784 |
| 133 | 206949_PM_s_at | ENSG00000160753 | RUSC1 | RUN and SH3 domain containing 1 | NESCA |
| 134 | 221269_PM_s_at | ENSG00000142669 | SH3BGRL3 | SH3 domain binding glutamate rich protein like 3 | |
| 135 | 223222_PM_at | ENSG00000125454 | SLC25A19 | solute carrier family 25 member 19 | DNC, MCPHA, MUP1, TPC |
| 136 | 213082_PM_s_at | ENSG00000130958 | SLC35D2 | solute carrier family 35 member D2 | SQV7L, UGTREL8 |
| 137 | 210057_PM_at | ENSG00000157106 | SMG1 | SMG1 phosphatidylinositol 3-kinase-related | ATX, KIAA0421, LIP |
| | | | | kinase | |
| 138 | 227052_PM_at | ENSG00000163683 | SMIM14 | small integral membrane protein 14 | C4ORF34, FLJ13289 |
| 139 | 1554986_PM_a_at | ENSG00000120451 | SNX19 | sorting nexin 19 | CHET8, KIAA0254 |
| 140 | 1563641_PM_a_at | ENSG00000167208 | SNX20 | sorting nexin 20 | SLIC-1, SLIC1 |
| 141 | 210985_PM_s_at | ENSG00000067066 | SP100 | SP100 nuclear antigen | |
| 142 | 217991_PM_x_at | ENSG00000157216 | SSBP3 | single stranded DNA binding protein 3 | CSDP, FLJ10355, SSDP, |
| | | | | | SSDP1 |
| 143 | 243981_PM_at | ENSG00000101109 | STK4 | serine/threonine kinase 4 | KRS2, MST1, YSK3 |
| 144 | 209306_PM_s_at | ENSG00000133789 | SWAP70 | SWAP switching B-cell complex 70 kDa subunit | KIAA0640, SWAP-70 |
| 145 | 221508_PM_at | ENSG00000135090 | TAOK3 | TAO kinase 3 | DPK, JIK, MAP3K18 |
| 146 | 212386_PM_at | ENSG00000196628 | TCF4 | transcription factor 4 | BHLHB19, E2-2, ITF2, |
| | | | | | SEF2-1B |
| 147 | 212762_PM_s_at | ENSG00000148737 | TCF7L2 | transcription factor 7-like 2 (T-cell specific, | TCF-4, TCF4 |
| | | | | HMG-box) | |
| 148 | 235461_PM_at | ENSG00000168769 | TET2 | tet methylcytosine dioxygenase 2 | FLJ20032, KIAA1546 |
| 149 | 1554608_PM_at | ENSG00000152291 | TGOLN2 | trans-golgi network protein 2 | TGN38, TGN46, TGN48, |
| | | | | | TGN51, TTGN2 |
| 150 | 54632_PM_at | ENSG00000115970 | THADA | THADA armadillo repeat containing | ARMC13, FLJ21877, GITA, |
| | | | | | KIAA1767, TRM732 |
| 151 | 218776_PM_s_at | ENSG00000137842 | TMEM62 | transmembrane protein 62 | FLJ23375 |
| 152 | 203671_PM_at | ENSG00000137364 | TPMT | thiopurine S-methyltransferase | |
| 153 | 202080_PM_s_at | ENSG00000182606 | TRAK1 | trafficking protein, kinesin binding 1 | KIAA1042, MILT1, OIP106 |
| 154 | 225294_PM_s_at | ENSG00000170043 | TRAPPC1 | trafficking protein particle complex 1 | BET5, MUM2 |
| 155 | 225870_PM_s_at | ENSG00000181029 | TRAPPC5 | trafficking protein particle complex 5 | MGC52424, TRS31 |
| 156 | 217979_PM_at | ENSG00000106537 | TSPAN13 | tetraspanin 13 | NET-6, TM4SF13 |
| 157 | 226175_PM_at | ENSG00000162222 | TTC9C | tetratricopeptide repeat domain 9C | MGC29649 |
| 158 | 203273_PM_s_at | ENSG00000114383 | TUSC2 | tumor suppressor 2, mitochondrial calcium | C3ORF11, FUS1, PAP, PDAP2 |
| | | | | regulator | |
| 159 | 202009_PM_at | ENSG00000247596 | TWF2 | twinfilin actin binding protein 2 | A6R, A6RP, PTK9L |
| 160 | 202333_PM_s_at | ENSG00000119048 | UBE2B | ubiquitin conjugating enzyme E2B | HHR6B, RAD6B, UBC2 |
| 161 | 208997_PM_s_at | ENSG00000175567 | UCP2 | uncoupling protein 2 (mitochondrial, proton | BMIQ4, SLC25A8 |
| | | | | carrier) | |
| 162 | 202090_PM_s_at | ENSG00000127540 | UQCR11 | ubiquinol-cytochrome c reductase, complex III | QCR10, UQCR |
| | | | | subunit XI | |
| 163 | 226033_PM_at | ENSG00000103404 | USP31 | ubiquitin specific peptidase 31 | KIAA1203 |
| 164 | 222799_PM_at | ENSG00000105875 | WDR91 | WD repeat domain 91 | HSPC049, SORF-1 |
| 165 | 210996_PM_s_at | ENSG00000108953 | YWHAE | tyrosine 3-monooxygenase/tryptophan 5- | FLJ45465 |
| | | | | monooxygenase activation protein, epsilon | |
| 166 | 243417_PM_at | ENSG00000180011 | PTGR3 | prostaglandin reductase 3 | MGC45594, PTGR-3, ZADH2 |
| 167 | 213286_PM_at | ENSG00000056097 | ZFR | zinc finger RNA binding protein | SPG71, ZFR1 |
| 168 | 206652_PM_at | ENSG00000132950 | ZMYM5 | zinc finger MYM-type containing 5 | MYM, ZNF198L1, ZNF237 |
| 169 | 208174_PM_x_at | ENSG00000169249 | ZRSR2 | zinc finger (CCCH type), RNA binding motif and | U2AF1-RS2, U2AF1L2, URP, |
| | | | | serine/arginine rich 2 | ZC3H22 |

### Results

A total of 77 patients receiving both pancreas and kidney transplant were included (simultaneous pancreas and kidney transplant “SPK” n=65; pancreas after kidney transplant “PAK” n=12). This Example evaluated all patients.

Dd-cfDNA increased significantly at 1 h and 24 h after transplantation compared to baseline (DO), most likely reflecting ischemia/reperfusion damage. No correlation with either donor demographics nor cold ischemia time was identified (p>0.05). In 52 cases a biopsy-related sample was obtained (Banff criteria: No rejection n=33; Indeterminate n=5; TCMR grade 1-3 n=15; ABMR n=4). In patients with stable graft function (no rejection during the first 12 months) dd-cfDNA decreased by D7 and remained low at B3 and B12 (D0 (0.27±0.14), 1 h (4.30±2.53), 24 h (2.41±1.79), D7 (0.65±0.47), B3 (0.47±0.28) and B12 (0.61±1.31)). See FIG. 1. However, in patients with biopsy proven acute rejection (T cell mediated rejection “TCMR;” 1.74±2.08 or antibody mediated rejection “ABMR;” 4.42±2.93; p<0.01), percent dd-cfDNA increased compared with no proven acute rejection biopsies (“N-BPAR” (no biopsy proven acute rejection); 0.45±0.77). Furthermore, a 1% dd-cfDNA threshold had a positive predictive value to detect 86% rejection, whereas the negative predictive value was 69%.

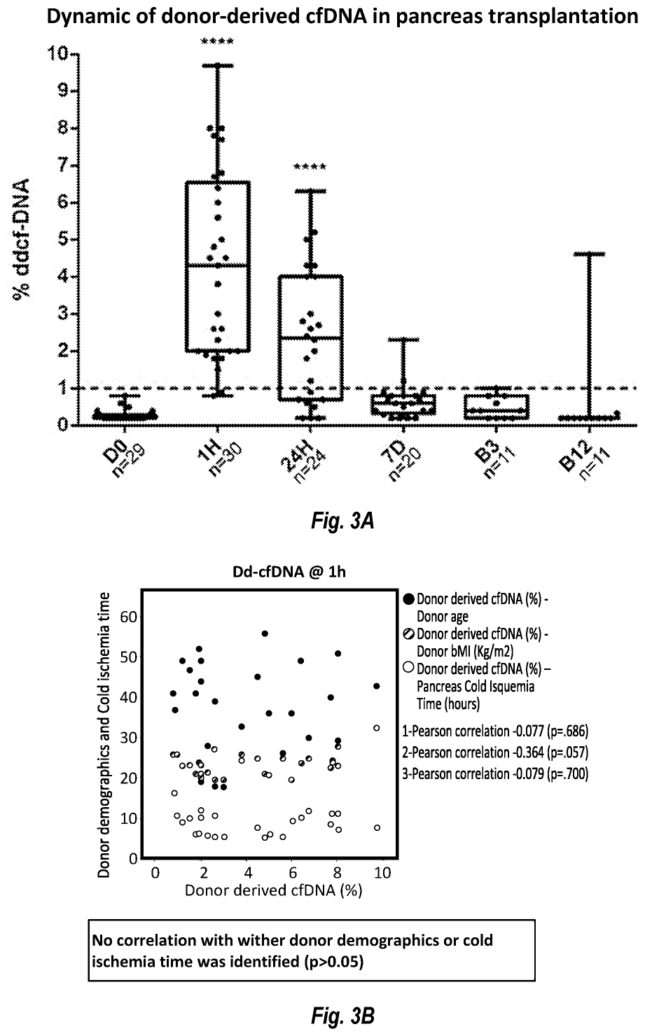

In the gene expression study, 75 patients with pancreas biopsy-related samples were included: N-BPAR=41; TCMR=23; ABMR=3 and undetermined=8. Dd-cfDNA increases early in the post transplant period. This is not related to traditional graft injury risk factors such as donor age or BMI or cold ischemia time. See FIGS. 3A-3B. Individual dd-cfDNA evaluation identified variations in patients who developed acute rejection during follow-up.

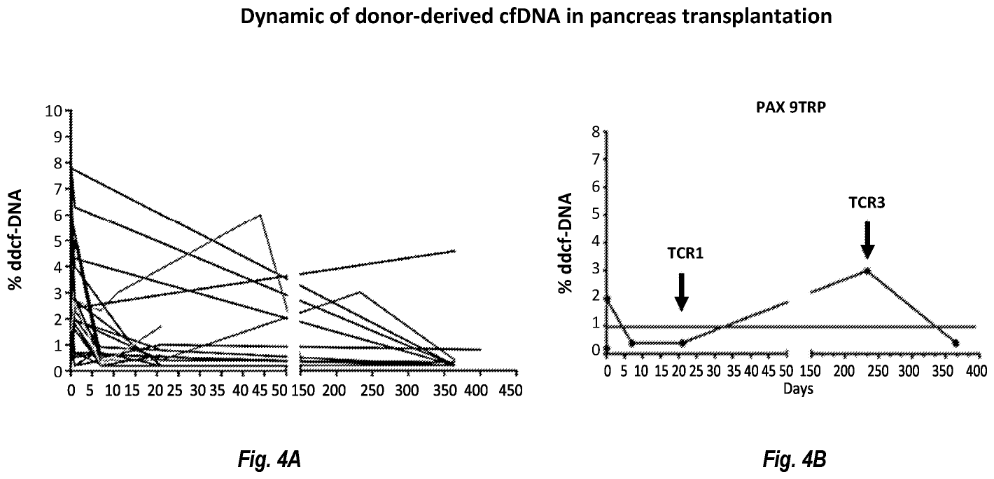

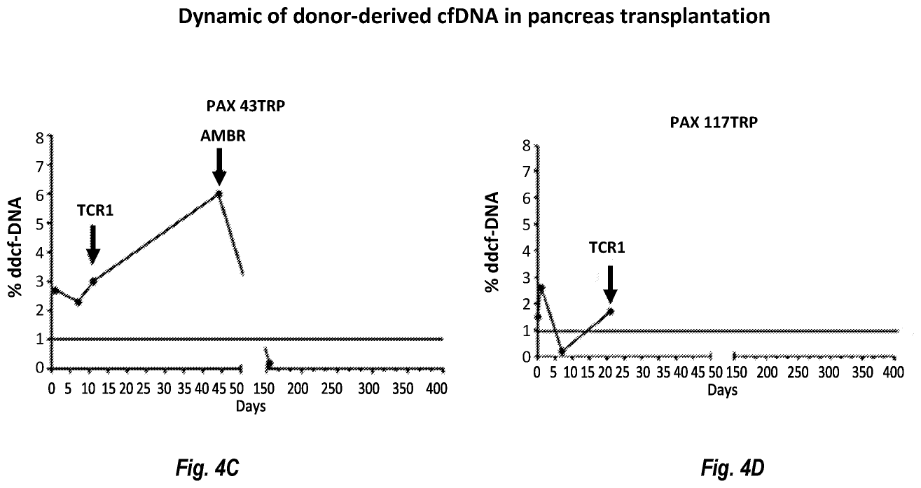

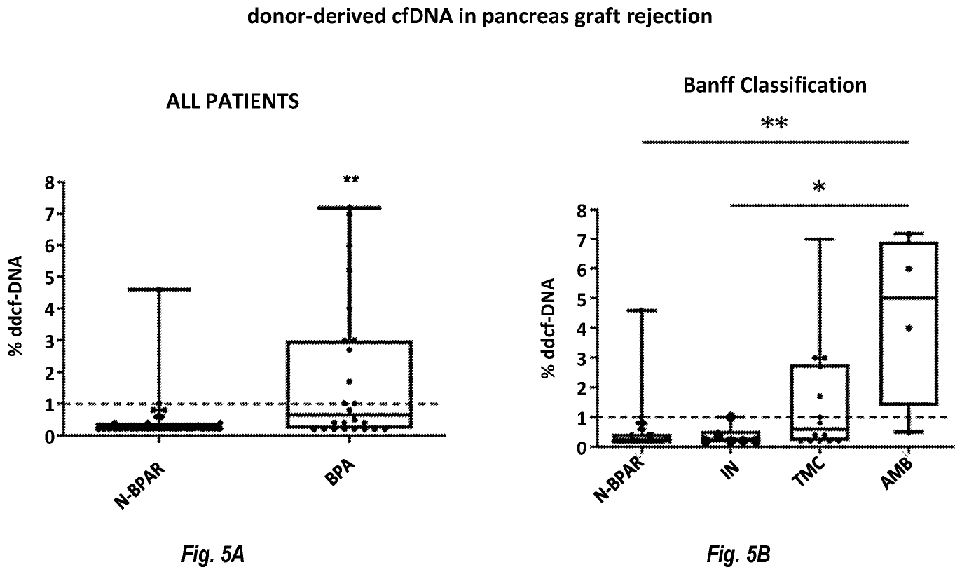

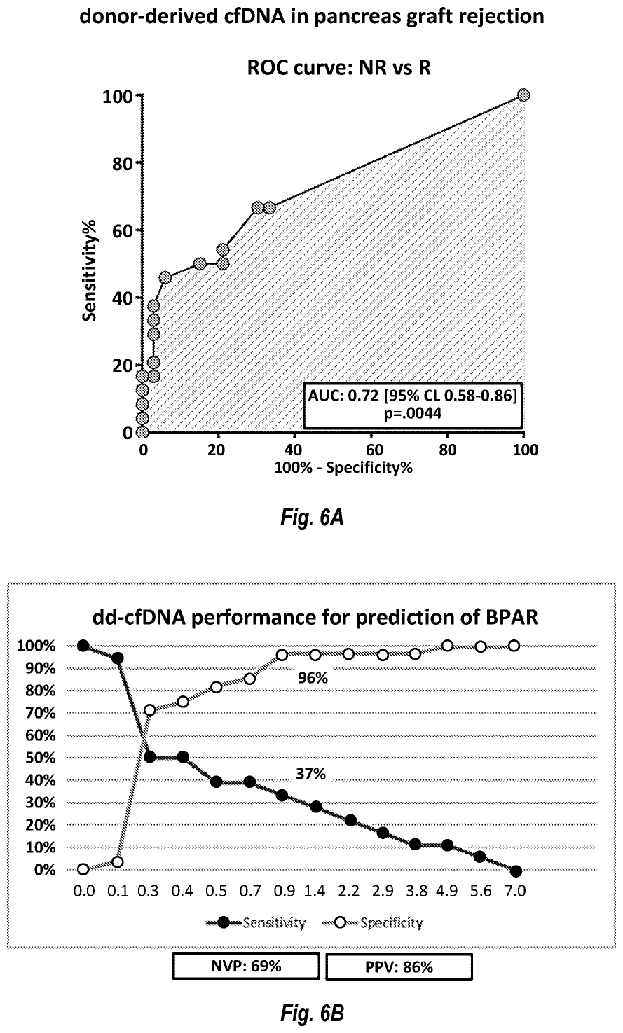

FIGS. 3-7 show the first dd-cfDNA dynamic analysis in pancreas transplant recipients, validating its application for longitudinal monitoring in clinical practice. As shown in FIGS. 3A-3B, the percentage of dd-cfDNA increased significantly within 1-24 hours of transplantation, as noted by the (****) symbols above the bar graph in FIG. 3A. It then reduced over time. As shown in FIG. 3B, the percentage of dd-cfDNA at 1 hour after transplantation did not correlate with either donor demographics or cold ischemia time. FIGS. 4A-4D show individual dd-cfDNA percentages over a period of 0-450 days post transplantation. Certain patients depicted in FIG. 4A showed no immunological events upon pancreas biopsy, in most cases having a dd-cfDNA percentage line below 1% after day 7, while others that showed immunological events, for example, are shown in FIGS. 4B-4D, and show dd-cfDNA values that rise above 1% after 7 days. One subject had a dd-cfDNA percentage value above 1% after day 7 despite not showing immunological events upon biopsy. Particular patient values are examined in FIG. 4B-4D, for subjects having biopsy-detected T cell mediated rejection (TCR) or antibody mediated rejection (ABMR). FIG. 5A shows a comparison between dd-cfDNA percentage in patients with no biopsy-proven acute rejection (N-BPAR) and those with biopsy-proven acute rejection (BPA) in the first 90 days following transplant. As shown in the figure, those with no rejection had a dd-cfDNA generally below 1%, while those with BPA showed a wide range of dd-cfDNA values, that were overall significantly higher than the subjects with no rejection, as indicated by the asterisks above the plot. FIG. 5B shows how the cases compare by Banff Classification, with no rejection (N-BPAR) and indeterminate (IN), for example, showing dd-cfDNA percentage below 1% in general, and T cell mediated rejection (TMC) and antibody mediated rejection (ABM) showing significantly higher dd-cfDNA levels. FIG. 6A-6B show the performance of the dd-cfDNA result, i.e., FIG. 6B shows the positive predictive value (PPV) of predicting rejection of 86%, and the negative predictive value (NPV) of predicting non-rejection of 69%.

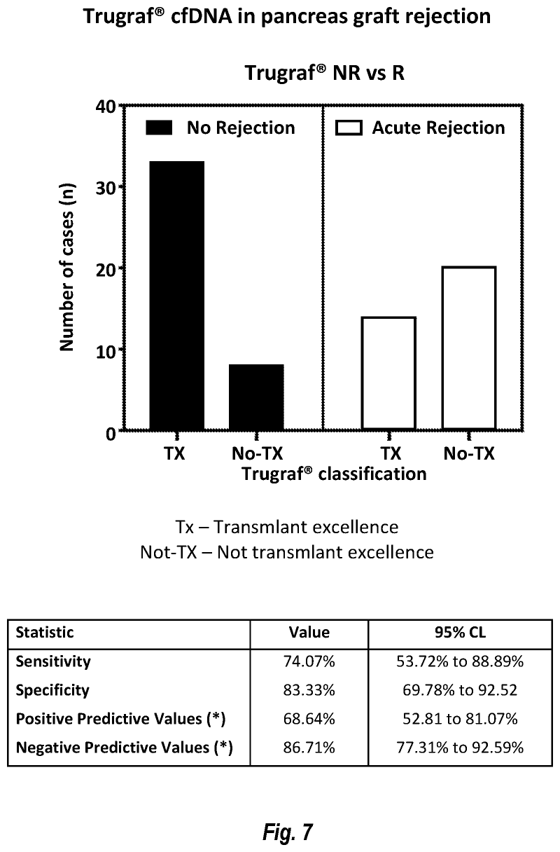

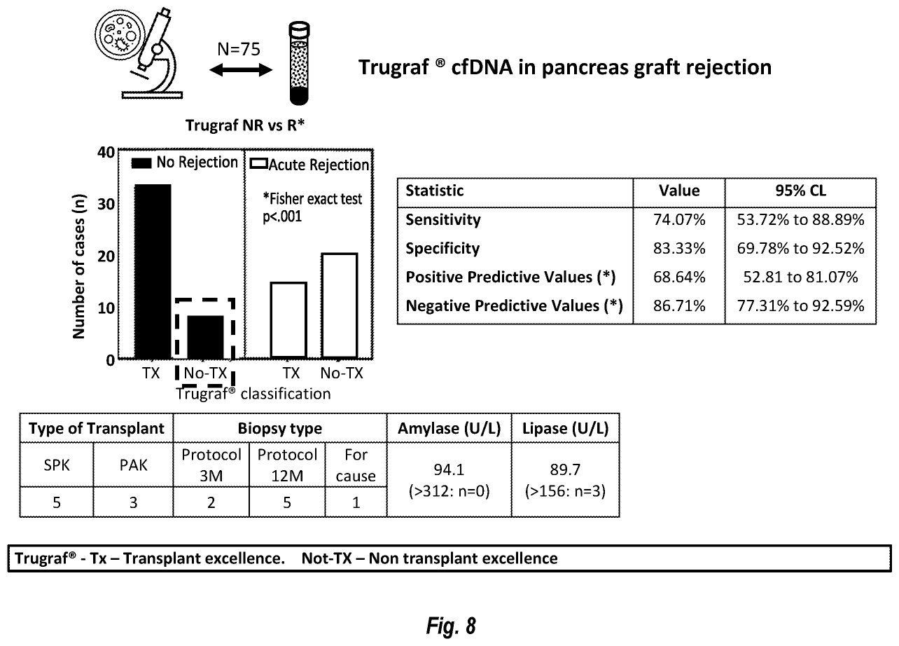

Combining these results with a gene expression profile assay may improve the performance for the diagnosis of subclinical rejection. For example, FIG. 7 shows the extent to which the TruGraf® gene expression assay was correctly able to predict non-rejection (i.e. TX) in these subjects in comparison with a pancreas biopsy result. As the left bars of the graph show, it was able to correctly predict non-rejection in most cases, with a negative predictive value of about 87%. FIGS. 8-16B show further data on how the TruGraf® gene expression assay correlates with rejection (no-TX) or non-rejection (TX) in the patient samples.

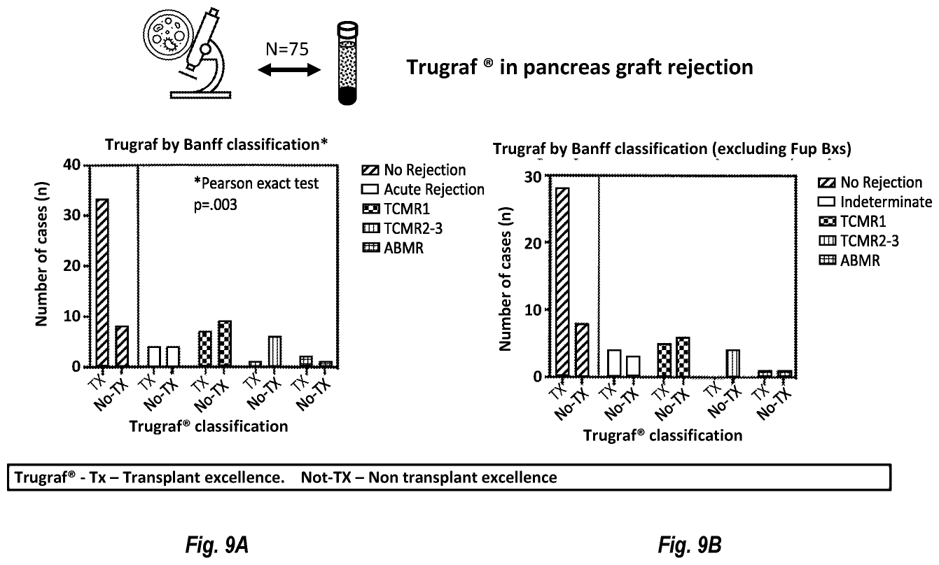

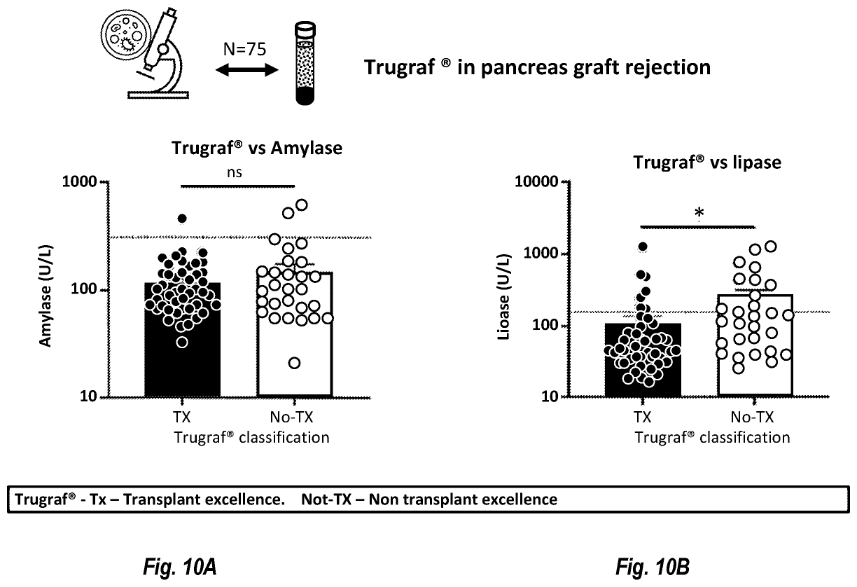

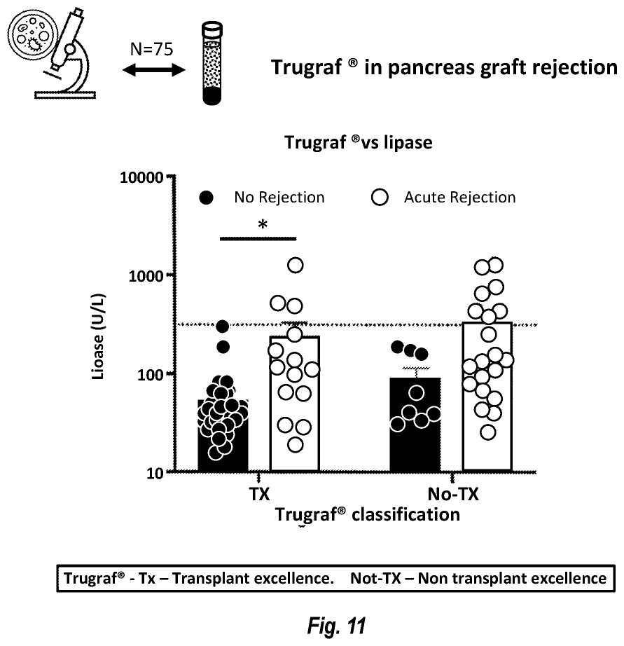

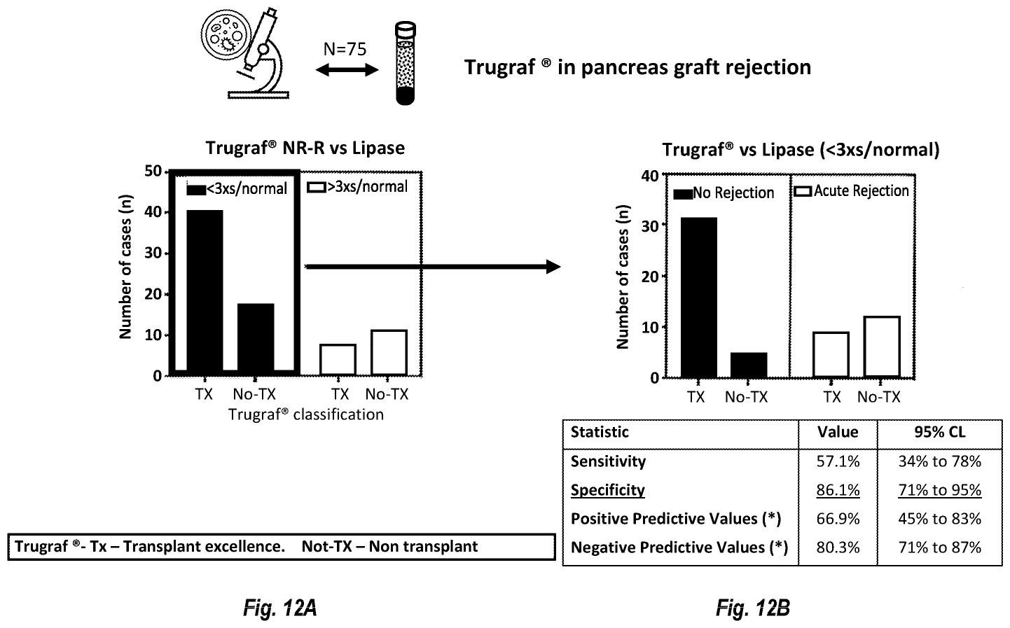

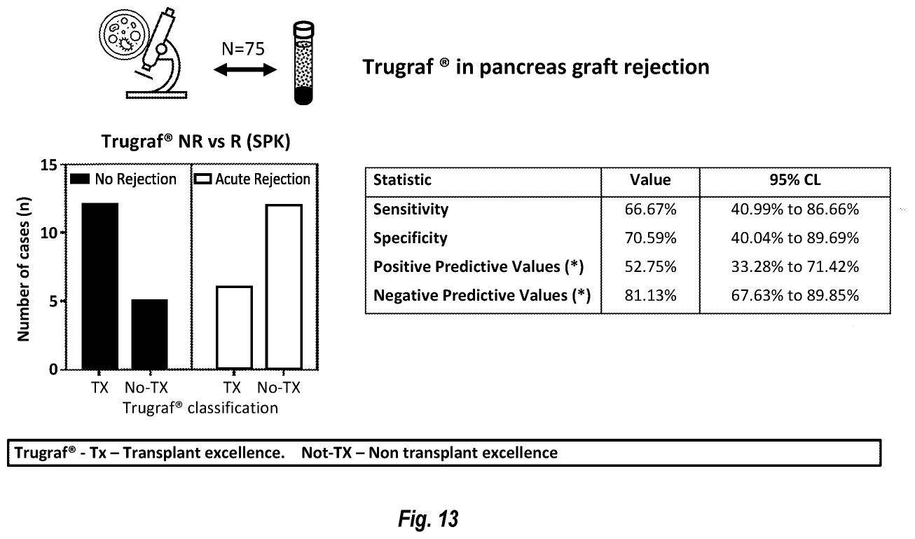

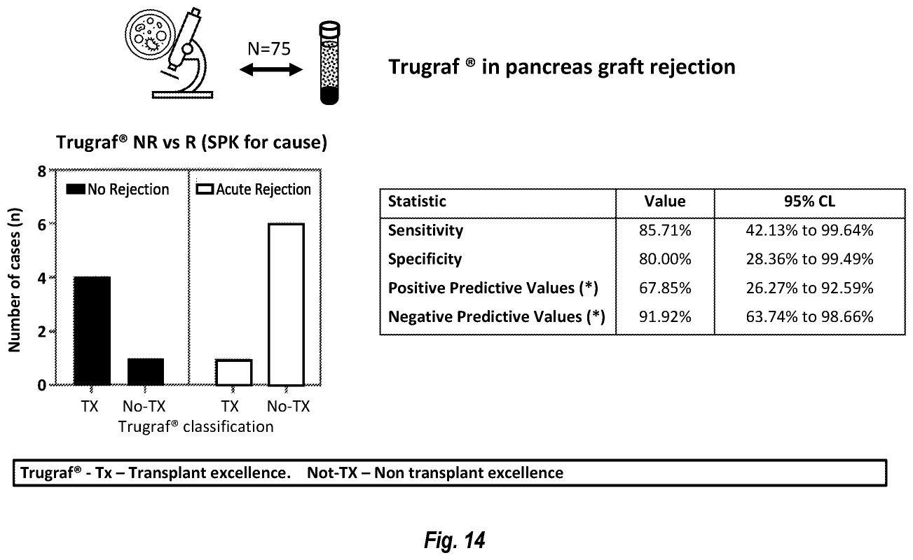

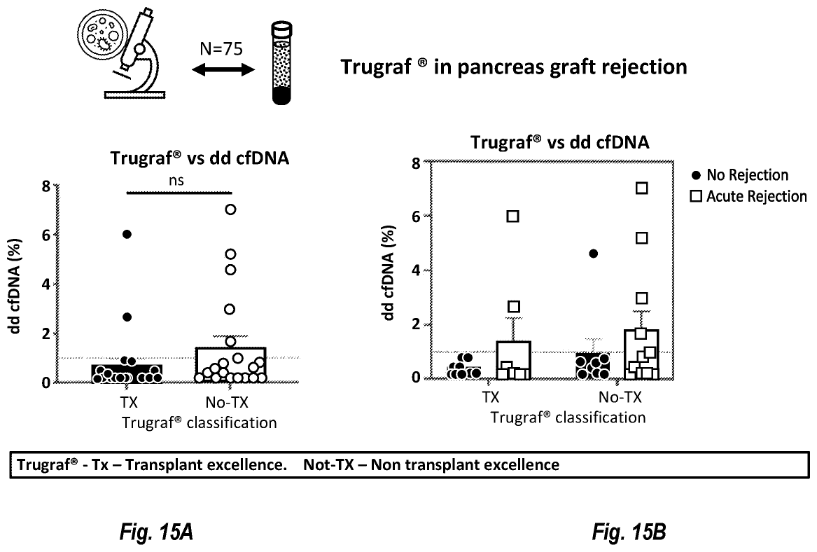

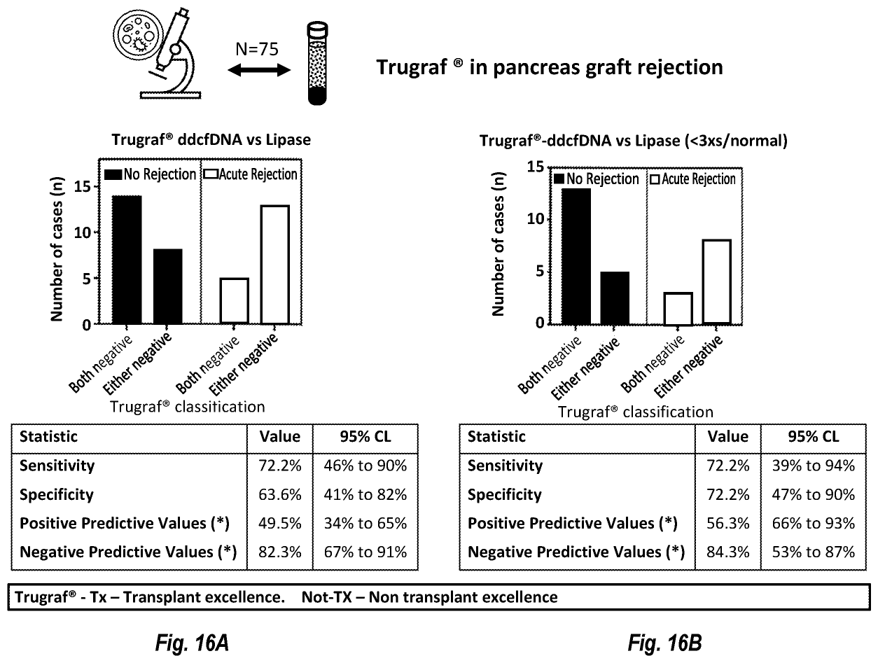

FIG. 8, for example, shows data from FIG. 7, highlighting those subjects in which the TruGraf® assay predicted rejection, but who, by a pancreas biopsy, were not found to have rejection (as shown in the bar below the graph). This included 8 subjects, 5 SPK and 3 PAK. The data also includes the amylase and lipase scores for the subjects as a comparison. FIGS. 9A-9B show the results by Banff Classification type, from no rejection on the left to ABMR on the far right. As shown in FIG. 9, the TruGraf® assay is able to correctly determine no rejection in most cases. FIGS. 10A-10B and 11 show comparisons of the TruGraf® assay with the currently used amylase and lipase assays (ns=not significant; * indicates statistical significance). The amylase and lipase thresholds distinguishing rejection are indicated by the dotted lines across each graph. FIG. 12A-B show the sensitivity of TruGraf® compared to lipase score. For example, FIG. 12A shows the TruGraf® classification in the two left bars for lipase scores less than three times normal (<3×s/normal) and in the two right bars more than 3 times normal (>3×s/normal). Further analysis of the TruGraf® classification for subjects with low lipase level less than three times normal is shown in FIG. 12B. FIG. 13 and FIG. 14 provide similar information for the subset of patients receiving a simultaneous pancreas kidney transplant (SPK). As shown in FIG. 14, TruGraf® had a 92% negative predictive value of acute rejection in those subjects. Further data comparing the performance of dd-cfDNA assays and TruGraf® are shown in FIGS. 15A-15B, while further comparisons of TruGraf® and dd-cfDNA together with lipase tests are shown in FIGS. 16A-16B.

### Example 2: Analysis of Simultaneous Pancreas Kidney Transplant Subjects

This example evaluates the subjects described above who had a simultaneous pancreas and kidney transplant (SPK).

### Material and Methods

### Study Design and Patient's Population

For this study we conducted a retrospective analysis using the stored patient samples. Plasma samples from simultaneous kidney-pancreas transplant recipients were collected pre-transplant (D0; n=26), 1 hour post-pancreas reperfusion (1 h; n=26), 24 hours after reperfusion (24 h; n=22), and at 7 days after transplant (7D; n=17). Afterwards plasma samples were collected at the time of pancreas graft biopsies. Biopsies were performed either for-cause (FC; n=14) or for surveillance (3 weeks (B3; n=9) and 12 months (B12; n=8) post-transplant). Blood collection was performed prior to the biopsy procedure. We excluded all samples in which a biopsy-matched blood sample was not obtained and those in which graft biopsy could not be performed, or sample was not suitable for histological diagnosis.

### Pancreas Graft Biopsies and Blood Samples

All biopsies were performed according to the center's protocol for pancreas graft monitoring. For cause biopsies were indicated if any of these conditions were present: i) >3×s (greater than three fold) increase in serum amylase or lipase; ii) hyperglycemia (fasting blood glucose >120 mg/dL); iii) de novo donor-specific antibodies (DSA); or iv) de novo anti-glutamic acid decarboxylase antibodies (GAD). Surveillance biopsies were performed at 3 weeks and at 12 months after transplantation, regardless of graft function, as well as at 4 weeks following the completion of the treatment for an acute rejection episode. Samples were obtained by ultrasound-guided percutaneous needle punch. Histological and immunohistochemical classification of pancreas graft biopsies was performed according to the 2011 Banff criteria.

Blood samples were obtained contemporaneously to pancreas graft biopsy and used to measure glucose (mg/dL), amylase (U/L), lipase (U/L), creatinine (mg/dL), C-Peptide (ng/mL), HbAlC (%), and anti-GAD (U/mL). Serum samples at time of biopsy were screened for HLA class I and II antibodies using the Lifecodes LifeScreen™ Deluxe flow bead assay (Immucor, Stamford, CT, USA). Antibody specificities, including the presence of DSA, were determined using the Lifecodes Single Antigen bead assay (Immucor, Stamford, CT, USA) in patients with positive screening for HLA antibodies.

### Donor-Derived Cell-Free DNA Analysis

Blood from pancreas transplant recipients was collected into PAXgene blood ccfDNA tubes (QIAGEN©), and plasma samples were obtained through double centrifugation following the manufacturer's instructions at the time of pancreas biopsies. All plasma samples were stored in a −80° C. freezer until sample processing.

The AlloNext™ assay distributed by Eurofins Genoma Group has been used to determine the dd-cfDNA percentage from the plasma samples. AlloNext™ uses NGS (Next Generation Sequencing) technique to measure differential allele contributions in a panel of more than 500 SNPs with high heterozygosity, low amplification error, and low linkage. Sequencing is performed at high resolution (sequencing depth >1000×, average 4000×). A custom NGS bioinformatics pipeline is used to align reads to the SNP regions, determine the contribution of donor-derived sequences to differentiate between donor and recipient cfDNA and calculate the percent of dd-cfDNA without requiring prior knowledge of donor genotypes. The minimum value reported by Allonext™ assay is 0.2%. Hence, all patients presenting dd-cfDNA under the detection of the assay are reported as 0.2%.

### Gene Expression Profile Analysis

At the time of pancreas biopsies blood was collected into PAXgene blood ccfDNA tubes (QIAGEN©), and plasma samples obtained through double centrifugation following the manufacturer's instructions. All plasma samples were stored in a −80° C. freezer until sample processing. Total RNA quantification was performed on a Thermo Scientific NanoDrop™ 8000 spectrophotometer. Using approximately 25 ng of total RNA, cDNA synthesis was performed by reverse transcription using the Fluidigm Reverse Transcription Master Mix kit. Pre-amplification was performed with 25% of the cDNA reaction mix using primer mixes supplied in the TaqMan® Gene Expression assays with 16 cycles of PCR. Following preamplification, samples were loaded into the interfluidic chip (IFC) using the Juno™ System. Gene expression quantification was performed with the same TaqMan® Gene Expression assays in the Fluidigm Biomark™ 192.24 Probe-Based GE IFC. Data analysis was performed using Fluidigm Real-Time PCR Analysis software v4.5.1 and Biomark Data Collection software v4.5.1. Cycle threshold values for multiple genes were analyzed through a custom algorithm which produces probability score and generates a TX (no rejection) or non-TX (rejection) result.

### Statistical Analysis

Data is represented as average±standard deviation (SD) for parametric continuous variables or as median [interquartile range 25-75] for nonparametric ones. Comparisons of median measurements were performed using Mann-Whitney U test, Pearson Correlations used for analysis between continuous variables, and Chi2 and Fisher exact test for comparison of binary variables. P value <0.05 was considered statistically significant. Sensitivity, specificity, positive predictive value (PPV), and negative predictive value (NPV) were calculated. Statistical analyses were performed in Prism version 9 (GraphPad LLC).

### Results

dd-cfDNA Dynamics Early after Pancreas Transplantation

A total of 26 simultaneous kidney-pancreas transplants for which a sample prior to transplantation was available were included in the longitudinal study. Recipients were mostly male (50%), already on dialysis at time of transplant (84.6%), with a mean age of 41.2 years, a body mass index of 22.5 kg/m2. Table 1 shows the detailed demographic and immunologic data of the longitudinal study cohort.

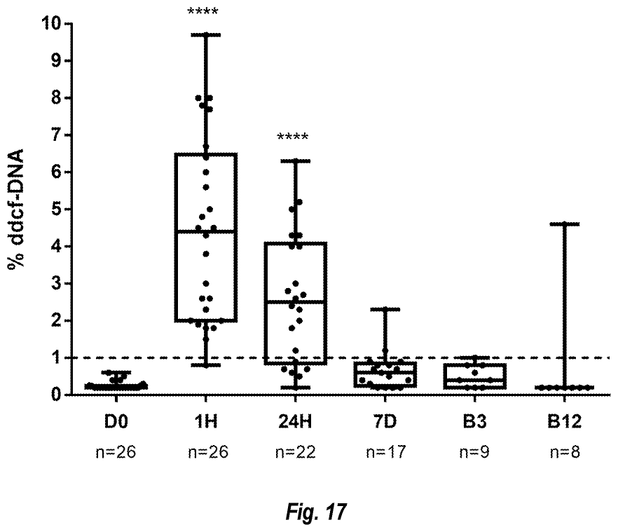

Prior to transplantation the median dd-cfDNA was of 0.2% [0.2-0.25], where 0.2% is the minimum value reported by the Allonext™ assay. Dd-cfDNA increased significantly in the first hour (4.4% [2-6.4]; p<0.0001; 1 h), with a subsequent decrease at 24 h (2.5% [0.8-4]; p<0.0001; 24 h) and 7 days after transplantation (0.6% [0.25-0.8]; 7D). In patients without immunological events during the first-year post-transplant (absence of kidney and/or pancreas acute rejection, or de novo DSA; n=11), dd-cfDNA stabilized <1% at week 3 (0.4% [0.2-0.8]; B3) and 12 months (0.2% [0.2-0.2]; B12) (FIG. 17).

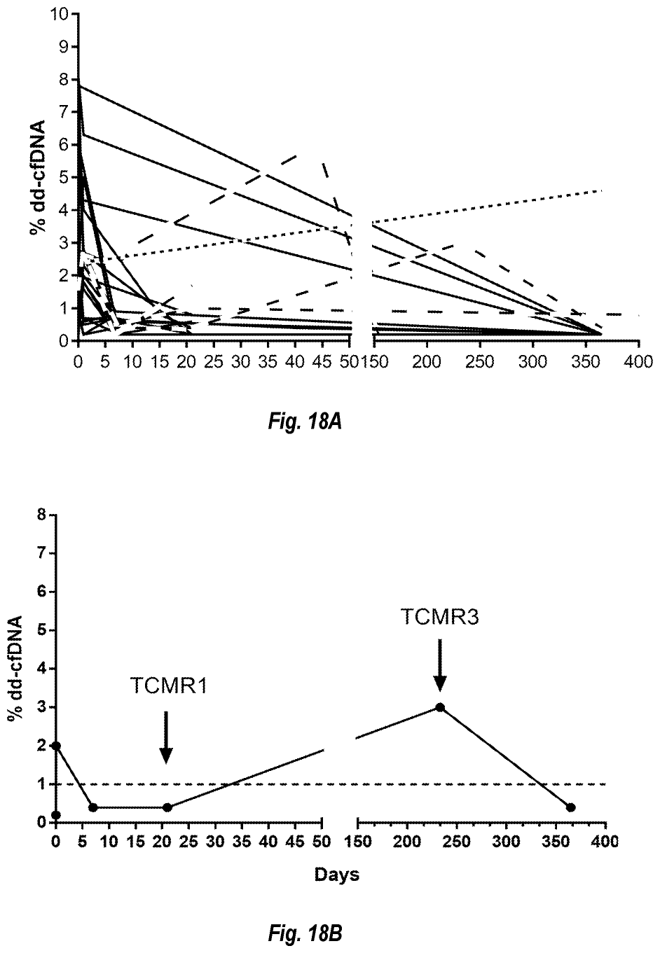

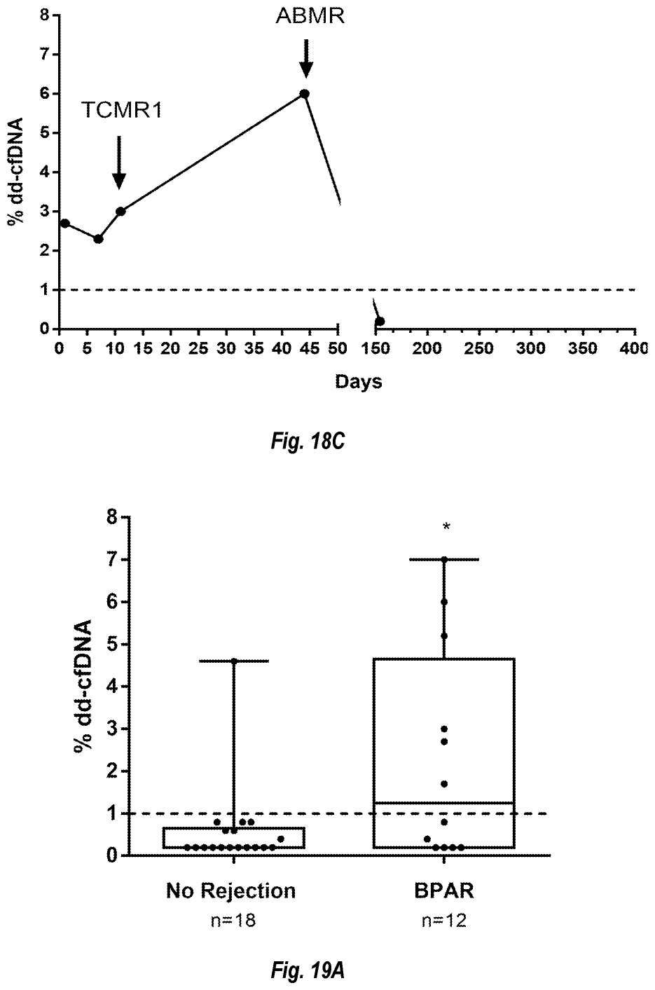

We further analyzed the dynamics of dd-cfDNA at the individual level in those patients with multiple samples collected prospectively. Overall, in patients without immunological events dd-cfDNA remained <1% from day 7 onwards (black lines FIG. 18A). In one single case (steadily rising line in FIG. 18A), dd-cfDNA increased >1% at 12 months, at which time patient presented normal graft function, normal amylase and lipase levels, and absence of acute rejection on protocol biopsy. In those patients who presented an acute rejection during the first year, there were intra-individual variations (variable lines in FIG. 18A). Most patients presented an early decrease in dd-cfDNA post-transplant, with subsequent increase at acute rejection episodes (FIG. 18B). In occasional cases, we identified dd-cfDNA to remain elevated despite the diagnosis and treatment for acute rejection (FIG. 18C).

### Dd-cfDNA Correlation of Ischemia-Reperfusion Injury

Ischemia-reperfusion injury (IRI) is a major cause of graft dysfunction in the early post-transplant period. Donor clinical and demographic characteristics and cold ischemia time (CIT) are well established risk factors for early graft dysfunction. Hence, we explored whether the dd-cfDNA determinations at 1 hour correlated with any of these characteristics. Dd-cfDNA correlated positively with donor body mass index (Pearson correlation 0.411; p=0.041), but failed to present any correlation with donor age (Pearson correlation-0.039; p=0.85), or cold ischemia time (Pearson correlation-0.053; p=0.81).

IRI is associated with an increased risk for acute rejection (AR) in the early post-transplant period. To address a potential utility to use dd-cfDNA as surrogate marker of IRI and subsequent AR, we analyzed the 1 h and 24 h dd-cfDNA from patients who presented an early pancreas AR episode (<90 days post-transplant) and compared to those without AR during the same period. Overall, there were no differences in dd-cfDNA at 1 h (4.58±0.81 vs 4.16±2.62; p=0.72) nor at 24 h (2.41±1.96 vs 2.72±1.79; p=0.74) in those with early AR compared to the remaining patients.

### Dd-cfDNA in Pancreas Acute Rejection

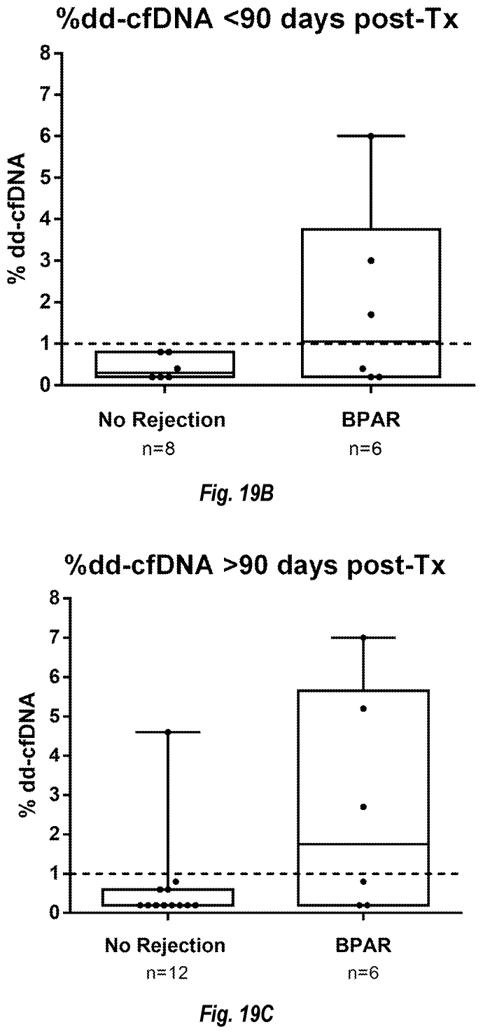

Among the 30-pancreas biopsy-matched plasma available for analysis, dd-cfDNA was significantly higher in patients with pancreas biopsy-proven acute rejection (P-BPAR; 1.2% [0.2-4.6]) compared to those with no rejection (0.2% [0.2-0.6]); p<0.037; FIG. 19A). When stratified by time post-transplant, dd-cfDNA was numerically but not statistically higher in patients with P-BPAR during the first 90 days post-transplant (P-BPAR; 1.05% [0.2-3.7] vs 0.3% [0.2-0.8], p=0.244, FIG. 19B), with similar results in P-BPAR diagnosed beyond the first 90 days (1.7% [0.2-5.6] vs 0.2% [0.2-0.6] in non-rejection; p=0.077; FIG. 19C).

### Gene Expression Profile in Pancreas Acute Rejection

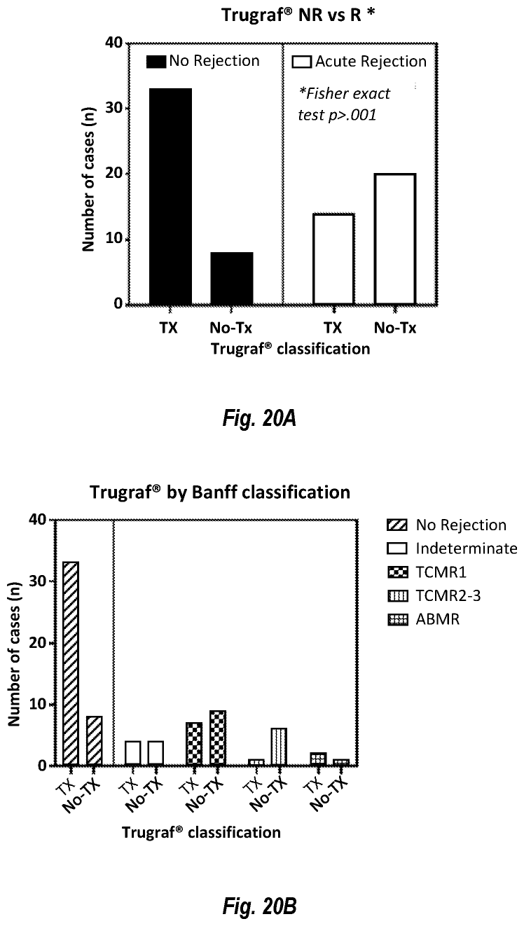

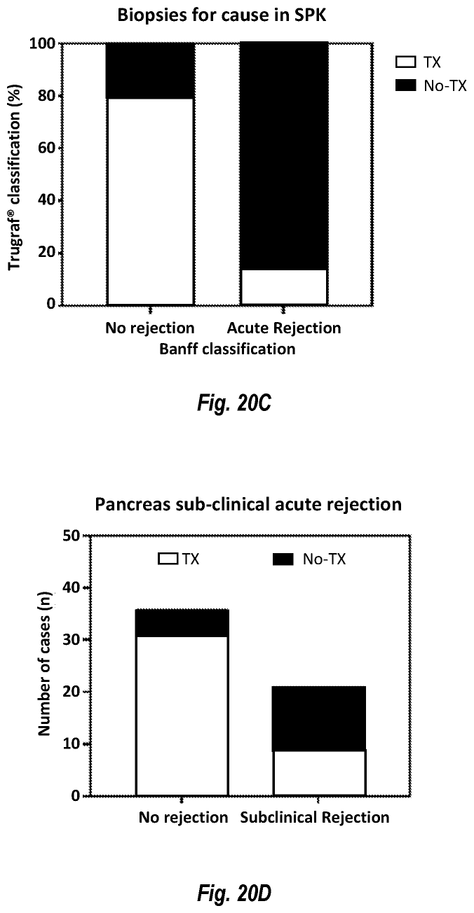

A total of 65 samples were available for analysis of plasma Gene Expression Profile (GEP) by Trugraf®. In the 31 cases with P-BPAR, the GEP was positive (No-TX—Not Transplant Excellence) in 58% (n=18) and negative (TX-Transplant Excellence) in 42% (n=13; FIG. 20A). Patients with P-BPAR were more likely to have a positive test (No-TX-58%) than those without rejection (No-TX 14.7%; p<0.001). Overall Trugraf® presented a sensitivity of 58.1% (95% CI 39.1-75.5) and a specificity of 85.3% (95% CI 68.9-95.1) for the diagnosis of acute rejection of the pancreas graft, with a negative predictive value (NPV) of 82.6% (95% CI 75.4-81.1) (Table 2). Biopsies with T-cell mediated rejection (TCMR, n=19) presented a higher frequency of No-TX (63.2%) compared to those without rejection (14.7%; p<0.001) (FIG. 20B).

We then decided to evaluate the ability of GEP to predict the diagnosis of acute rejection in biopsies performed for cause (n=12; FIG. 20C), hence excluding those biopsies performed for surveillance. Trugraf® was positive in six patients with P-BPAR (85%), presenting a sensitivity of 85.7% and a specificity of 80.0% for the diagnosis of acute rejection, and a negative predictive value of 91% (Table 2).

We then compared the GEP to the current available biomarkers. Patients with No-TX (i.e., rejection) had higher lipase levels (133 U/mL [65-402]) compared to those with TX (i.e, no rejection) (45 U/mL [30-77]; p=0.001), without major differences in the amylase levels (112 U/mL [75-185] vs 96 U/mL [68-142]; p=0.62). To explore the potential clinical benefit to diagnose pancreas subclinical rejections, we evaluated the performance of Trugraf® in patients with lipase less than three times (<3×s) normal at time of biopsy. Of the 19 patients with subclinical rejection, 53% presented a No-TX (FIG. 20D). Trugraf® presented a sensitivity of 52.6% (95% CI 29-76) but a specificity of 93.1% (95% CI 77-99) for the diagnosis of subclinical rejection of the pancreas graft, with NPV of 82.1% (95% CI 74-88) (Table 2).

### Combined Dd-cfDNA and GEP Performance

In 40 patients there was enough biopsy-matched sample volume to perform both dd-cfDNA and GEP. We explored the performance of combining these two biomarkers to the diagnosis pancreas acute rejection. Overall, there was no significant correlation between the Trugaf® classification (TX vs No-TX) and the dd-cfDNA levels. Thirteen patients with P-BPAR (72.2%) presented either test positive (No-TX or dd-cfDNA ≥1%). The combination of both diagnostic techniques presented a sensitivity of 72.2% (95% CI 46-90) and a specificity of 63.6% (95% 41-82%), with a negative predictive value of 82.3% (67-91%).

### Discussion

In this Example, we evaluated the performance of donor-derived cfDNA and gene expression profile in various settings after pancreas transplantation, using longitudinally collected plasma samples at different timepoints after transplantation. Studies with novel biomarkers in pancreas transplantation are of particular relevance due to peculiarities of this type of transplantation. On one hand, pancreas graft is believed to be more prone to ischemia-reperfusion injury. Herein we quantified donor-derived cfDNA at various time points in the immediate post-transplant period to explore the correlation of this biomarker during this early period with post-transplant with graft outcomes, such as acute rejection. On the other hand, diagnosis of pancreas graft rejection is often challenging due to the technical difficulties of performing a pancreas transplant biopsy, and to the low specificity of currently available biomarkers. The number of studies demonstrating the utility of these blood-based molecular biomarkers in pancreas transplantation is scarce, and often lack from pancreas graft biopsy correlation. By including pancreas biopsy-matched only samples we aimed at reducing the bias induced by clinical diagnosis of acute rejection.

The dynamic of dd-cfDNA following pancreas transplantation observed in this study is similar to what has been described in other abdominal organ transplants dd-cfDNA. Dd-cfDNA presented an increase in the hour after organ reperfusion, followed by a progressive reduction thereafter with stable values <1% by the third week post-transplant. This early increase in dd-cfDNA has been postulated to be correlated with the degree of ischemia-reperfusion injury. Older donor age, prolonged cold ischemia time, and hemodynamic instability prior to organ procurement, are traditional risk factors for pancreas graft thrombosis and primary non-function. Nonetheless, none of these risk factors correlated with the ratio of dd-cfDNA in the immediate post-transplant period. Though the study is not powered to definitely exclude such correlation, it unveils that other variables may influence the ischemia-reperfusion injury of the pancreas graft other than any of these criteria alone. This hypothesis is supported by the fact that pre-procurement scoring systems have failed to correlate with graft outcomes. The global urgence to increase the pancreas donor pool (i.e., increasing donor age or DCD donors) warrants further studies using dd-cfDNA in the immediate post-transplant period to aid in the refinement of donor selection criteria.

Understanding the dynamics of a biomarker after transplantation is of particular relevance to be able to accurately define its performance for diagnosing acute rejection. In a transversal cohort, we had previously identified that dd-cfDNA accuracy for the diagnosis of acute rejection in pancreas transplantation improved significantly beyond day 45 after transplantation. The previous study lacked the longitudinal follow-up herein presented, in which we could identify that by week 3 post-transplant patients with stable pancreas graft function had dd-cfDNA <1%. Moreover, we identified that patients with P-BPAR from month 3 onwards post-transplant had higher dd-cfDNA ratio compared to those without rejection. It is important to highlight that samples at each timepoint were not available from all patients included the study, the sample size is small, and there only three cases of antibody mediated rejection, which altogether limits the extrapolation of the results. Nonetheless, they corroborate what was also observed in other organs, in which dd-cfDNA may be a useful tool for the diagnosis of pancreas acute rejection beyond the early post-transplant period.

Biomarkers with continuous values, such as dd-cfDNA, provide additional benefits for graft monitoring such as intra-individual variations in longitudinal follow-up. These variations may be useful when monitoring graft dysfunction, diagnosing acute rejection, or monitoring response to treatment. In those patients in which there were samples from at least 3 different timepoints during the longitudinal follow-up, we could identify that in those who developed an acute rejection during the first year, there was an observed increase in dd-cfDNA value relative to the previous determination.

Molecular expression of graft biopsies was introduced in recent updates to Banff classification as a subject for future research. Recently, several studies in renal transplantation showed that the blood transcriptomic profile could reflect the inflammatory and immunologic tissue status during the clinical course of the recipient, and in particular during acute rejection episodes. We explored for the first time the utility of gene expression profile from blood-based samples demonstrating its ability to predict acute rejection in pancreas transplantation. The gene expression profile presented good specificity and negative predictive values for the diagnosis of acute rejection. Furthermore, the gene expression profile demonstrated an ability to diagnose subclinical pancreas rejection. Finally, in biopsies performed for cause in SPK TX recipients, Trugaf® presented a negative predictive value of 91%. Analysis of for cause biopsies in retrospective studies are biased by the clinical criteria to perform the biopsy, however. Despite this limitation, the high sensitivity (86%) and specificity (80%) may aid clinicians on their decision to treat for an acute rejection episode whenever a graft biopsy cannot be performed and may lead to a reduction in the number of surveillance biopsies.

Donor-derived cell-free DNA has good accuracy for the diagnosis of antibody mediated rejection. T cell mediated rejection is the most frequent rejection during the first year after pancreas transplantation, however. Herein we observed that the gene expression profile demonstrated good accuracy for diagnosis of T-cell mediated rejection. Interestingly, when both molecular techniques were combined, the overall performance was not superior to that of the gene expression profile alone. Two major factors might have contributed to this difference. Firstly, the number of patients analyzed for the performance of combined biomarkers was almost half of those included for the gene expression profile alone. This was due to the retrospective nature of the study, which was limited to the available plasma volume for analysis. Secondly, in the combined analysis there was only one case of antibody-mediated rejection (ABMR). As stated above, dd-cfDNA is particularly accurate for the diagnosis of ABMR. The low number of ABMR cases in the analysis likely limited dd-cfDNA from improving the performance of the gene expression profile alone.

The authors would like to highlight an important limitation to this study. Most patients received a simultaneous kidney transplant from the same donor, but matched kidney biopsy data was not available. Elevation of both donor-derived cfDNA and gene expression profile in kidney graft dysfunction is well described in literature. Thus, it is not possible to exclude the potential confounding results due cfDNA released by the kidney graft, which could be experiencing rejection. The absence of concomitant kidney graft biopsies preclude that clinicians did not suspect of simultaneous rejection of both grafts. Nonetheless, gene expression profile has demonstrated good performance for the diagnosis of subclinical kidney acute rejection, therefore one cannot exclude that the false positive results observed in patients without pancreas acute rejection might be secondary to an ongoing subclinical kidney graft rejection. Despite this limitation, a major strength of our study relies on the fact that only pancreas-biopsy matched samples were included, maximizing the extrapolation of the performance regarding the specificity and negative predictive value of the biomarkers.

TABLE 1
Study demographics and baseline characteristics
of the study cohort.

| | Demographics | |
|---|---|---|
| Recipient | | |
| Age (years) Median [IQR] | 41.2 | [34.3-50.8] |
| Gender (Male, n) | 13 | (50%) |
| DM type | | |
| T1D (n) | 24 | (92.3%) |
| T2D (n) | 2 | (7.7%) |
| DM vintage (years) Median [IQR] | 25 | [20.2-30] |
| BMI Median [IQR] | 22.5 | [20.4-25] |
| Type of dialysis | | |
| Preemptive (n) | 4 | (15.4%) |
| Hemodialysis (n) | 20 | (76.9%) |
| Peritoneal dialysis (n) | 2 | (7.7%) |
| HLA Mismatches | 5.0 | [4.0-6.0] |
| (ABDR) Median [IQR] | | |
| Donor | | |
| Age (years) Median [IQR] | 37.5 | [25-46.7] |
| Gender (Male, n) | 12 | (46.2%) |
| BMI Median [IQR] | 23.2 | [20.8-25.3] |
| Cause of death | | |
| Cerebrovascular disease (%) | 50% | |
| Trauma (%) | 41.6% | |
| Anoxic encephalopathy (%) | 4.2% | |
| Other (%) | 4.2% | |
| Donor type | | |
| DBD (%) | 100% | |
| DCD (%) | 0% | |
| Pancreas cold ischemia time | 8.6 | [7.1-11.1] |

| | |
|---|---|
| (hours) Median [IQR] | |
| Induction Immunosuppression | |
| Thymoglobulin (%) | 88.5% |
| Basiliximab (%) | 11.5% |

BMI—Body Mass Index;
DBD—Donor after brain death;
DCD—Donor after cardiocirculatory death;
DM—Diabetes Mellitus;
IQR—Interquartile range;
SPK—Simultaneous kidney-pancreas transplantation;
T1D—Type 1 Diabetes Mellitus;
T2D—Type 2 Diabetes Mellitus

TABLE 2
Performance of gene expression profile alone for
the diagnosis of pancreatic transplant rejection.

| Diagnostic | | T cell mediated | For cause | Sub-clinical |
|---|---|---|---|---|
| performance | Overall | rejection | biopsies | rejection |
| Sensitivity | 58.1% | 63.1% | 85.7% | 52.6% |
| (95% confidence | (39.1% to | (38.4% to | (42.1% to | (28.9% to |
| interval) | 75.5%) | 83.7%) | 99.6%) | 75.5%) |
| Specificity | 85.3% | 85.3% | 80.0% | 93.1% |
| (95% confidence | (68.9% to | (68.9% to | (28.4% to | (77.2% to |
| interval) | 95.1%) | 95.0%) | 99.45%) | 99.2%) |
| Positive | 62.9% | 64.8% | 67.89% | 76.6% |
| Predictive Value | (41.6% to | (43.3% to | (26.3% to | (44.5% to |
| (95% confidence | 80.0%) | 81.6%) | 92.6%) | 93.0%) |
| interval) | | | | |
| Negative | 82.6% | 84.4% | 91.9% | 82.1% |
| Predictive Value | (75.4% to | (74.7% to | (63.7% to | (73.8% to |
| (95% confidence | 88.1%) | 90.8%) | 98.67%) | 88.2%) |
| interval) | | | | |

### Example 3: Analysis of TruGraf® Gene Expression in Simultaneous Pancreas Kidney Transplant Recipients

Further experiments were performed to determine genes in the Trugraf® assay that show different RNA transcript expression levels in non-TX samples compared to TX samples. There were 38 samples out of the 65 SPK samples described in Examples 1 and 2 in which a plasma gene expression profile was compared to a pancreatic biopsy. Fifteen subjects had no rejection (TX), 4 subjects were indeterminate, 16 subjects had T cell mediated rejection, and 3 subjects had antibody mediated rejection. There were a number of particular genes in the TruGraf® assay that were statistically upregulated at the mRNA level in the subjects with rejection, as shown in Table 3 below.

TABLE 3
Trugraf ® gene transcripts with higher expression in non-TX recipients compared to TX recipients

| | Log2 | std error | Lower conf. lim. | Upper conf. lim. | Linear | Lower conf. lim. | Upper conf. lim. | | | |
|---|---|---|---|---|---|---|---|---|---|---|
| Transcript | fold change | (log2) | (log2) | (log2) | fold change | (linear) | (linear) | P-val | Gene.sets | probe.ID |
| IGHG2 | 3.98 | 1.21 | 1.62 | 6.35 | 15.8 | 3.07 | 81.3 | 0.00218 | Plasma cells | ENST00000390545.1:374 |
| IGHG1 | 3.84 | 1.21 | 1.48 | 6.2 | 14.3 | 2.79 | 73.7 | 0.00297 | Plasma cells | ENST00000390542.1:240 |
| IGHG4 | 3.54 | 1.15 | 1.29 | 5.8 | 11.6 | 2.44 | 55.5 | 0.00397 | Plasma cells | ENST00000390543.1:729 |
| IGHA1 | 3.43 | 0.983 | 1.51 | 5.36 | 10.8 | 2.84 | 41.1 | 0.00129 | GRIT3, Plasma cells | ENST00000641837.1:604 |
| IGLC1 | 3.02 | 0.614 | 1.81 | 4.22 | 8.1 | 3.52 | 18.6 | 1.94E−05 | Plasma cells | ENST00000390321.1:178 |
| IGHG3 | 3 | 0.996 | 1.05 | 4.96 | 8.02 | 2.07 | 31 | 0.00468 | Plasma cells | ENST00000390551.1:548 |
| IGKC | 2.82 | 0.895 | 1.06 | 4.57 | 7.06 | 2.09 | 23.8 | 0.00329 | Plasma cells | ENST00000390237.1:230 |
| IRF4 | 1.96 | 0.453 | 1.07 | 2.85 | 3.89 | 2.1 | 7.19 | 0.000114 | IFNg Signalling, IL-4 and IL-13 signaling, | NM_001195286.1:1365 |
| | | | | | | | | | Th17 Differentiation, Type I Interferon | |
| | | | | | | | | | Signaling | |
| IL7R | 1.88 | 0.583 | 0.74 | 3.02 | 3.68 | 1.67 | 8.13 | 0.00265 | Cytokine Signaling, IL-7 signaling, | NM_002185.3:201 |
| | | | | | | | | | Vesicle-mediated transport | |
| CD96 | 1.61 | 0.556 | 0.517 | 2.7 | 3.05 | 1.43 | 6.49 | 0.0065 | Adaptive Immune System, TCMR-RATs | NM_005816.4:428 |
| SLAMF6 | 1.59 | 0.459 | 0.685 | 2.49 | 3 | 1.61 | 5.6 | 0.00144 | Adaptive Immune System, GRIT2, | NM_001184714.2:1805 |
| | | | | | | | | | TCMR-RATs | |
| SLAMF7 | 1.58 | 0.631 | 0.34 | 2.81 | 2.98 | 1.27 | 7.03 | 0.0172 | Adaptive Immune System, GRIT3 | NM_021181.4:787 |
| GZMK | 1.55 | 0.607 | 0.358 | 2.74 | 2.92 | 1.28 | 6.66 | 0.0152 | Cytotoxicity, QCAT, TCMR-RATs | NM_002104.2:634 |
| IGHM | 1.5 | 0.542 | 0.436 | 2.56 | 2.82 | 1.35 | 5.89 | 0.0089 | Plasma cells | ENST00000637539.1:583 |
| ZAP70 | 1.46 | 0.545 | 0.39 | 2.53 | 2.75 | 1.31 | 5.76 | 0.0111 | Adaptive Immune System, Cytotoxicity, | NM_001079.3:1175 |
| | | | | | | | | | NF-kappaB Signaling, T-cell Receptor | |
| | | | | | | | | | Signaling | |
| CD3E | 1.45 | 0.375 | 0.712 | 2.18 | 2.73 | 1.64 | 4.54 | 0.000457 | Adaptive Immune System, Co- | NM_000733.2:75 |
| | | | | | | | | | Stimulation by CD28 Family, T-cell | |
| | | | | | | | | | Receptor Signaling | |
| CD79A | 1.43 | 0.332 | 0.781 | 2.08 | 2.7 | 1.72 | 4.23 | 0.000119 | Adaptive Immune System, B-cell | NM_001783.3:104 |
| | | | | | | | | | Receptor Signaling, BAT | |
| CXCR6 | 1.41 | 0.446 | 0.537 | 2.29 | 2.66 | 1.45 | 4.87 | 0.00315 | Chemokine Signaling, QCAT, TCMR- | NM_006564.1:97 |
| | | | | | | | | | RATs, TCMR21 | |
| CD3D | 1.4 | 0.468 | 0.485 | 2.32 | 2.64 | 1.4 | 4.99 | 0.00494 | Adaptive Immune System, Co- | NM_000732.4:110 |
| | | | | | | | | | Stimulation by CD28 Family, QCAT, T- | |
| | | | | | | | | | cell Receptor Signaling, TCMR, TCMR- | |
| | | | | | | | | | RATs, TCMR NHP, TCMR21, TOL NHP, | |
| | | | | | | | | | Vesicle-mediated transport | |
| MIR155HG | 1.38 | 0.478 | 0.438 | 2.31 | 2.6 | 1.35 | 4.97 | 0.00673 | | NR_001458.3:360 |
| CTSW | 1.37 | 0.549 | 0.293 | 2.45 | 2.58 | 1.23 | 5.45 | 0.0174 | Cytotoxicity | NM_001335.3:1075 |
| SLA | 1.35 | 0.529 | 0.309 | 2.38 | 2.54 | 1.24 | 5.22 | 0.0154 | T-cell Receptor Signaling, TCMR, TCMR- | NM_001045556.2:980 |
| | | | | | | | | | RATs | |
| IL2RG | 1.33 | 0.553 | 0.243 | 2.41 | 2.51 | 1.18 | 5.32 | 0.0217 | Cytokine Signaling, IL-2 family signaling, | NM_000206.1:595 |
| | | | | | | | | | IL-4 and IL-13 signaling, IL-7 signaling, | |
| | | | | | | | | | Th17 Differentiation, Th2 | |
| | | | | | | | | | Differentiation | |
| CXCR4 | 1.32 | 0.487 | 0.363 | 2.27 | 2.49 | 1.29 | 4.83 | 0.0104 | Chemokine Signaling, Lymphocyte | NM_003467.2:1335 |
| | | | | | | | | | Trafficking | |
| ISG20 | 1.29 | 0.364 | 0.572 | 2 | 2.44 | 1.49 | 4 | 0.00115 | GRIT3, tCRM, Type I Interferon | NM_002201.5:864 |
| | | | | | | | | | Signaling | |
| IL2RB | 1.26 | 0.505 | 0.269 | 2.25 | 2.39 | 1.2 | 4.75 | 0.0174 | Cytokine Signaling, IL-2 family signaling, | NM_000878.3:350 |
| | | | | | | | | | QCAT, Th2 Differentiation | |
| CCL5 | 1.26 | 0.396 | 0.48 | 2.03 | 2.39 | 1.39 | 4.09 | 0.0031 | Chemokine Signaling, Cytosolic DNA | NM_002985.2:277 |
| | | | | | | | | | Sensing, GRIT2, IL-10 Signaling, NLR | |
| | | | | | | | | | Signaling, Oxidative Stress, Pre8, TCMR | |
| | | | | | | | | | NHP, TNF Family Signaling, Toll-like | |
| | | | | | | | | | Receptor Signaling | |
| PRDM1 | 1.26 | 0.359 | 0.561 | 1.97 | 2.4 | 1.47 | 3.91 | 0.00118 | exhaustion, GRIT2, Mac Trained | NM_182907.1:310 |
| CCR5 | 1.24 | 0.489 | 0.283 | 2.2 | 2.36 | 1.22 | 4.59 | 0.0156 | Chemokine Signaling, IL-10 Signaling, | NM_000579.1:2730 |
| | | | | | | | | | TCMR-RATs, TCMR NHP | |
| CCR2 | 1.23 | 0.424 | 0.398 | 2.06 | 2.34 | 1.32 | 4.17 | 0.00632 | Chemokine Signaling, IL-10 Signaling, | NM_001123041.2:743 |
| | | | | | | | | | Innate Immune System, IRITD5 | |
| AOAH | 1.22 | 0.451 | 0.331 | 2.1 | 2.32 | 1.26 | 4.29 | 0.0107 | Innate Immune System, TCMR-RATs | NM_001637.3:1303 |
| HLA-DQB1 | 1.22 | 0.412 | 0.415 | 2.03 | 2.33 | 1.33 | 4.09 | 0.00532 | Adaptive Immune System, Co- | NM_002123.2:820 |
| | | | | | | | | | Stimulation by CD28 Family, GRIT1, | |
| | | | | | | | | | GRIT3, IFNg Signalling, MHC Class II | |
| | | | | | | | | | Antigen Presentation, T-cell Receptor | |
| | | | | | | | | | Signaling | |
| IDO1 | 1.2 | 0.484 | 0.251 | 2.15 | 2.3 | 1.19 | 4.43 | 0.0181 | ABMR-RATs, ABMRpm, DSAprob, | NM_002164.5:52 |
| | | | | | | | | | GRIT3, IFNg Signalling, M1 Mac, Pre8 | |
| GZMA | 1.16 | 0.463 | 0.254 | 2.07 | 2.24 | 1.19 | 4.19 | 0.0168 | GRIT3, QCAT | NM_006144.2:155 |
| IKZF1 | 1.14 | 0.39 | 0.378 | 1.91 | 2.21 | 1.3 | 3.75 | 0.00585 | NOTCH Signaling | NM_006060.4:225 |
| KLRB1 | 1.13 | 0.39 | 0.361 | 1.89 | 2.18 | 1.28 | 3.71 | 0.00654 | Adaptive Immune System, TCMR NHP | NM_002258.2:85 |
| TNFSF8 | 1.12 | 0.409 | 0.319 | 1.92 | 2.17 | 1.25 | 3.79 | 0.00951 | TCMR, TNF Family Signaling, TNFR2 | NM_001244.3:399 |
| | | | | | | | | | non-canonical NF-kB pathway | |
| CD8A | 1.09 | 0.421 | 0.266 | 1.92 | 2.13 | 1.2 | 3.78 | 0.0137 | Adaptive Immune System, QCAT, T-cell | NM_001768.6:2029 |
| | | | | | | | | | Receptor Signaling, TCMR-RATs, TCMR | |
| | | | | | | | | | NHP, TCMR21 | |
| IL16 | 1.09 | 0.349 | 0.407 | 1.78 | 2.13 | 1.33 | 3.42 | 0.0035 | Cytokine Signaling | NM_004513.4:1262 |
| HLA-DRA | 1.07 | 0.383 | 0.32 | 1.82 | 2.1 | 1.25 | 3.54 | 0.00828 | ABMR-RATs, Adaptive Immune System, | NM_019111.3:335 |
| | | | | | | | | | GRIT1, GRIT3, IFNg Signalling, MHC | |
| | | | | | | | | | Class II Antigen Presentation, T-cell | |
| | | | | | | | | | Receptor Signaling | |
| CD84 | 1.07 | 0.368 | 0.346 | 1.79 | 2.1 | 1.27 | 3.45 | 0.00633 | TCMR, TCMR-RATs | NM_001184879.1:28 |
| BTK | 1.05 | 0.489 | 0.0882 | 2 | 2.06 | 1.06 | 4.01 | 0.0392 | Adaptive Immune System, B-cell | NM_000061.1:570 |
| | | | | | | | | | Receptor Signaling, Innate Immune | |
| | | | | | | | | | System, MHC Class I Antigen | |
| | | | | | | | | | Presentation, NF-kappaB Signaling, Rho | |
| | | | | | | | | | GTPase signaling, Toll-like Receptor | |
| | | | | | | | | | Signaling | |
| NKG7 | 1.05 | 0.46 | 0.146 | 1.95 | 2.07 | 1.11 | 3.86 | 0.0288 | NK, Pre8, QCAT | NM_005601.3:633 |
| SELPLG | 1.04 | 0.472 | 0.115 | 1.97 | 2.06 | 1.08 | 3.91 | 0.034 | | NM_003006.4:969 |
| CD45R0 | 1.04 | 0.404 | 0.251 | 1.84 | 2.0 | 1.19 | 3.57 | 0.0141 | Lymphocyte Trafficking, T-cell Receptor | NM_080921.3:258 |
| | | | | | | | | | Signaling | |
| PTPRC | 1.02 | 0.36 | 0.311 | 1.72 | 2.02 | 1.24 | 3.3 | 0.00773 | Adaptive Immune System, Innate | NM_080921.2:90 |
| | | | | | | | | | Immune System, IRRAT, Neutrophil | |
| | | | | | | | | | degranulation, T-cell Receptor Signaling | |
| ITGAX | 1.01 | 0.387 | 0.256 | 1.77 | 2.02 | 1.19 | 3.42 | 0.0127 | Complement Receptors, IL-4 and IL-13 | NM_000887.4:872 |
| | | | | | | | | | signaling, Innate Immune System, | |
| | | | | | | | | | TCMR NHP | |
| CX3CR1 | 0.99 | 0.319 | 0.364 | 1.62 | 1.99 | 1.29 | 3.07 | 0.00375 | ABMR-RATs, ABMRpm, AMR, CAMR | NM_001337.3:1040 |
| | | | | | | | | | NHP, Chemokine Signaling, DSA10, | |
| | | | | | | | | | DSAST, DSAST-Non-Endo, IRITD5, TOL | |
| | | | | | | | | | NHP | |
| IL10RA | 0.972 | 0.377 | 0.234 | 1.71 | 1.96 | 1.18 | 3.27 | 0.014 | Cytokine Signaling, IL-10 Signaling, Treg | NM_001558.2:150 |
| | | | | | | | | | Differentiation | |
| CD48 | 0.955 | 0.415 | 0.141 | 1.77 | 1.94 | 1.1 | 3.41 | 0.0273 | Cytotoxicity | NM_001778.3:43 |
| ST8SIA4 | 0.937 | 0.333 | 0.285 | 1.59 | 1.91 | 1.22 | 3.01 | 0.00786 | TCMR, TCMR-RATs | NM_005668.4:695 |
| IRF8 | 0.935 | 0.403 | 0.145 | 1.72 | 1.91 | 1.11 | 3.3 | 0.0261 | GRIT2, GRIT3, IFNg Signalling, Type I | NM_002163.2:253 |
| | | | | | | | | | Interferon Signaling | |
| FCGR2B | 0.932 | 0.364 | 0.219 | 1.65 | 1.91 | 1.16 | 3.13 | 0.0148 | Adaptive Immune System, B-cell | NM_001002273.1:870 |
| | | | | | | | | | Receptor Signaling | |
| LST1 | 0.914 | 0.438 | 0.0558 | 1.77 | 1.88 | 1.04 | 3.42 | 0.044 | ABMR-RATs, IFNg Signalling | NM_007161.3:371 |
| PTPN22 | 0.907 | 0.263 | 0.392 | 1.42 | 1.88 | 1.31 | 2.68 | 0.00145 | Adaptive Immune System, T-cell | NM_012411.5:342 |
| | | | | | | | | | Receptor Signaling | |
| ITGA4 | 0.9 | 0.396 | 0.123 | 1.68 | 1.87 | 1.09 | 3.2 | 0.0292 | Adaptive Immune System, Extracellular | NM_000885.4:975 |
| | | | | | | | | | Matrix Organization, GRIT2, | |
| | | | | | | | | | Lymphocyte Trafficking | |
| INPP5D | 0.899 | 0.294 | 0.322 | 1.48 | 1.86 | 1.25 | 2.78 | 0.00421 | Adaptive Immune System, B-cell | NM_001017915.2:2694 |
| | | | | | | | | | Receptor Signaling, IL-2 family signaling, | |
| | | | | | | | | | T-cell Receptor Signaling, tCRM | |
| BIRC3 | 0.861 | 0.368 | 0.14 | 1.58 | 1.82 | 1.1 | 2.99 | 0.0249 | Apoptosis, Innate Immune System, | NM_182962.1:3 |
| | | | | | | | | | IRITD5, NF-kappaB Signaling, NLR | |
| | | | | | | | | | Signaling, Programmed cell death, TNF | |
| | | | | | | | | | Family Signaling, TNFR2 non-canonical | |
| | | | | | | | | | NF-kB pathway, Toll-like Receptor | |
| | | | | | | | | | Signaling | |
| IL27RA | 0.838 | 0.355 | 0.141 | 1.53 | 1.79 | 1.1 | 2.9 | 0.024 | Cytokine Signaling | NM_004843.3:1640 |
| TPSAB1/B2 | 0.805 | 0.317 | 0.184 | 1.43 | 1.75 | 1.14 | 2.69 | 0.0156 | Extracellular Matrix Organization | NM_003294.3:579 |
| FGD2 | 0.803 | 0.364 | 0.0898 | 1.52 | 1.74 | 1.06 | 2.86 | 0.0338 | Rho GTPase signaling, TCMR | NM_173558.3:1119 |
| CD74 | 0.802 | 0.342 | 0.132 | 1.47 | 1.74 | 1.1 | 2.77 | 0.0245 | ABMR-RATs, Adaptive Immune System, | NM_001025158.2:442 |
| | | | | | | | | | GRIT1, GRIT3, MHC Class II Antigen | |
| | | | | | | | | | Presentation, TCMR NHP | |
| HLA-DPB1 | 0.8 | 0.344 | 0.125 | 1.47 | 1.74 | 1.09 | 2.78 | 0.0259 | ABMR-RATs, Adaptive Immune System, | NM_002121.5:800 |
| | | | | | | | | | Co-Stimulation by CD28 Family, GRIT3, | |
| | | | | | | | | | IFNg Signalling, MHC Class II Antigen | |
| | | | | | | | | | Presentation, T-cell Receptor Signaling | |
| CXCL16 | 0.8 | 0.309 | 0.195 | 1.41 | 1.74 | 1.14 | 2.65 | 0.0138 | Chemokine Signaling, QCMAT | NM_001100812.1:850 |
| HLA-DRB3 | 0.792 | 0.362 | 0.0826 | 1.5 | 1.73 | 1.06 | 2.83 | 0.0352 | Adaptive Immune System, Co- | NM_022555.3:698 |
| | | | | | | | | | Stimulation by CD28 Family, IFNg | |
| | | | | | | | | | Signalling, MHC Class II Antigen | |
| | | | | | | | | | Presentation, T-cell Receptor Signaling | |
| KLRK1 | 0.786 | 0.274 | 0.25 | 1.32 | 1.72 | 1.19 | 2.5 | 0.0068 | Cytotoxicity | NM_007360.1:760 |
| PIK3CG | 0.783 | 0.34 | 0.116 | 1.45 | 1.72 | 1.08 | 2.73 | 0.0272 | NK-ADCC | NM_002649.2:2125 |
| MEOX1 | 0.759 | 0.328 | 0.116 | 1.4 | 1.69 | 1.08 | 2.64 | 0.0265 | AMR, ENDAT | NM_001040002.1:1710 |
| HLA-DPA1 | 0.749 | 0.346 | 0.0713 | 1.43 | 1.68 | 1.05 | 2.69 | 0.037 | ABMR-RATs, Adaptive Immune System, | NM_033554.3:305 |
| | | | | | | | | | Co-Stimulation by CD28 Family, GRIT3, | |
| | | | | | | | | | IFNg Signalling, MHC Class II Antigen | |
| | | | | | | | | | Presentation, T-cell Receptor Signaling | |
| SDC1 | 0.728 | 0.206 | 0.325 | 1.13 | 1.66 | 1.25 | 2.19 | 0.00113 | Extracellular Matrix Organization, | NM_002997.4:2680 |
| | | | | | | | | | Oxidative Stress | |
| HLA-DMB | 0.712 | 0.345 | 0.0365 | 1.39 | 1.64 | 1.03 | 2.62 | 0.0461 | Adaptive Immune System, GRIT1, | NM_002118.3:20 |
| | | | | | | | | | GRIT3, MHC Class II Antigen | |
| | | | | | | | | | Presentation, TCMR-RATs | |
| CD4 | 0.71 | 0.305 | 0.113 | 1.31 | 1.64 | 1.08 | 2.48 | 0.0255 | Adaptive Immune System, Co- | NM_000616.4:975 |
| | | | | | | | | | Stimulation by CD28 Family, Innate | |
| | | | | | | | | | Immune System, T-cell Receptor | |
| | | | | | | | | | Signaling, TCMR, TCMR NHP, Vesicle- | |
| | | | | | | | | | mediated transport | |
| B2M | 0.705 | 0.26 | 0.196 | 1.21 | 1.63 | 1.15 | 2.32 | 0.0101 | ABMR-RATs, Adaptive Immune System, | NM_004048.2:235 |
| | | | | | | | | | GRIT1, IFNg Signalling, Innate Immune | |
| | | | | | | | | | System, MHC Class I Antigen | |
| | | | | | | | | | Presentation | |
| ITGB6 | 0.695 | 0.338 | 0.0319 | 1.36 | 1.62 | 1.02 | 2.56 | 0.0473 | AKI, Extracellular Matrix Organization | NM_000888.3:1090 |
| TLR2 | 0.687 | 0.262 | 0.174 | 1.2 | 1.61 | 1.13 | 2.3 | 0.0126 | Adaptive Immune System, Innate | NM_003264.3:2402 |
| | | | | | | | | | Immune System, MHC Class I Antigen | |
| | | | | | | | | | Presentation, Neutrophil degranulation, | |
| | | | | | | | | | QCMAT, Toll-like Receptor Signaling | |
| F3 | 0.683 | 0.27 | 0.153 | 1.21 | 1.61 | 1.11 | 2.32 | 0.016 | GRIT3, Oxidative Stress | NM_001993.3:1030 |
| HLA-F | 0.674 | 0.297 | 0.0917 | 1.26 | 1.6 | 1.07 | 2.39 | 0.0294 | ABMR-RATs, Adaptive Immune System, | NM_001098479.1:575 |
| | | | | | | | | | DSAprob, GRIT3, IFNg Signalling, MHC | |
| | | | | | | | | | Class I Antigen Presentation, TCMR- | |
| | | | | | | | | | RATs, Type I Interferon Signaling | |
| ARHGDIB | 0.603 | 0.262 | 0.0892 | 1.12 | 1.52 | 1.06 | 2.17 | 0.0273 | IRITD5, Rho GTPase signaling | NM_001175.5:1082 |
| STAT4 | 0.592 | 0.272 | 0.0581 | 1.13 | 1.51 | 1.04 | 2.18 | 0.0364 | Cytokine Signaling, IL-12 family | NM_003151.2:789 |
| | | | | | | | | | signaling, IL-2 family signaling, QCAT, | |
| | | | | | | | | | Th1 Differentiation | |
| TAP2 | 0.568 | 0.261 | 0.0558 | 1.08 | 1.48 | 1.04 | 2.11 | 0.0364 | Adaptive Immune System, GRIT2, | NM_000544.3:909 |
| | | | | | | | | | GRIT3, MHC Class I Antigen | |
| | | | | | | | | | Presentation | |
| IFNAR2 | 0.558 | 0.198 | 0.17 | 0.947 | 1.47 | 1.13 | 1.93 | 0.00778 | Cytotoxicity, NLR Signaling, Toll-like | NM_000874.3:631 |
| | | | | | | | | | Receptor Signaling, Type I Interferon | |
| | | | | | | | | | Signaling | |
| ALOX5 | 0.556 | 0.193 | 0.178 | 0.934 | 1.47 | 1.13 | 1.91 | 0.00665 | Eosinophils, IL-1 family signaling, Innate | NM_000698.2:735 |
| | | | | | | | | | Immune System, M2 Mac, Neutrophil | |
| | | | | | | | | | degranulation | |
| PIK3CD | 0.547 | 0.226 | 0.104 | 0.991 | 1.46 | 1.07 | 1.99 | 0.0208 | Adaptive Immune System, | NM_005026.3:2978 |
| | | | | | | | | | Angiogenesis, B-cell Receptor Signaling, | |
| | | | | | | | | | Cell Cycle Regulation, Chemokine | |
| | | | | | | | | | Signaling, Cytotoxicity, IL-2 family | |
| | | | | | | | | | signaling, Lymphocyte Trafficking, | |
| | | | | | | | | | mTOR, T-cell Receptor Signaling, TNF | |
| | | | | | | | | | Family Signaling, Toll-like Receptor | |
| | | | | | | | | | Signaling | |
| IL18 | 0.533 | 0.181 | 0.178 | 0.888 | 1.45 | 1.13 | 1.85 | 0.00564 | Cytokine Signaling, Cytosolic DNA | NM_001562.3:364 |
| | | | | | | | | | Sensing, GRIT2, IL-1 family signaling, IL- | |
| | | | | | | | | | 10 Signaling, IL-4 and IL-13 signaling, | |
| | | | | | | | | | NLR Signaling | |
| TNFAIP3 | 0.531 | 0.212 | 0.115 | 0.947 | 1.44 | 1.08 | 1.93 | 0.0171 | Innate Immune System, NF-kappaB | NM_006290.2:260 |
| | | | | | | | | | Signaling, NLR Signaling, Oxidative | |
| | | | | | | | | | Stress, TNF Family Signaling | |
| C1S | 0.53 | 0.247 | 0.0456 | 1.01 | 1.44 | 1.03 | 2.02 | 0.0388 | Complement Components, GRIT2, | NM_001734.2:775 |
| | | | | | | | | | GRIT3, Innate Immune System | |
| CD58 | 0.503 | 0.151 | 0.208 | 0.799 | 1.42 | 1.15 | 1.74 | 0.00198 | Innate Immune System | NM_001779.2:478 |

Further analysis also showed that Trugraf® text results statistically correlated with the presence of graft infiltrating immune cells, including CDD45+ cells, CD4+ T cells, CD8+ T cells, cytotoxic cells, macrophages, and neutrophils.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of distinguishing rejection from non-rejection in a pancreatic transplant recipient, the method comprising
a. obtaining a sample from the pancreatic transplant recipient;
b. obtaining cell-free DNA (cfDNA) from said sample;
c. determining the level of donor derived cell-free DNA (dd-cfDNA) in the cfDNA; and
d. distinguishing rejection from non-rejection in the recipient based upon results of the dd-cfDNA, wherein rejection in the recipient is indicated by a level of dd-cfDNA at or above a pre-determined threshold value.

2. A method of distinguishing rejection from non-rejection in a pancreatic transplant recipient, the method comprising
a. obtaining a sample from the pancreatic transplant recipient;
b. obtaining mRNA from the sample;
c. determining the expression level of at least one mRNA transcript, wherein the at least one mRNA transcript shows significantly different expression levels in pancreatic transplant rejection compared to pancreatic transplant non-rejection subjects; and
d. distinguishing rejection from non-rejection in the recipient based upon the expression level of at least one mRNA transcript, wherein rejection in the recipient is indicated by result of a trained algorithm based on the expression level of the at least one mRNA transcript indicating rejection or non-rejection, wherein the algorithm compares the expression profile of the at least one mRNA transcript of the recipient to the expression profile of pancreatic transplant subjects with and without rejection.

3. A method of distinguishing rejection from non-rejection in a pancreatic transplant recipient, the method comprising
a. obtaining a blood, plasma, or serum sample from the pancreatic transplant recipient;
b. obtaining cell-free DNA (cfDNA) and mRNA from the sample;
c. determining (i) the level of donor derived cell-free DNA (dd-cfDNA) in the cfDNA and (ii) the expression level of at least one mRNA transcript, wherein the at least one mRNA transcript shows significantly different expression levels in pancreatic transplant rejection compared to pancreatic transplant non-rejection subjects; and
d. distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) result of a trained algorithm based on the expression level of the at least one mRNA transcript indicating rejection or non-rejection, wherein the algorithm compares the expression profile of the at least one mRNA transcript of the recipient to the expression profile of pancreatic transplant subjects with and without rejection.

4. The method of any one of claims 1-3, wherein the recipient is a pancreatic and kidney transplant recipient, such as a pancreas after kidney transplant recipient or a simultaneous pancreas and kidney transplant recipient.

5. The method of any one of claim 1 or 3-4, wherein rejection in the recipient is indicated by predetermined threshold value of dd-cfDNA of ≥0.5%, ≥0.6%, ≥0.7%, ≥0.8%, ≥0.9%, ≥1%, ≥1.2%, ≥1.5%, or ≥2%.

6. The method of any one of claim 1 or 3-4, wherein rejection in the recipient is indicated by a pre-determined threshold value of dd-cfDNA of ≥0.7%, optionally wherein determining the dd-cfDNA level utilizes data from recipient genotype information.

7. The method of any one of claim 1 or 3-4, wherein rejection in the recipient is indicated by a pre-determined threshold value of dd-cfDNA of ≥1.0 or >1.0, optionally wherein determining the dd-cfDNA level utilizes data from recipient genotype information.

8. The method of any one of claims 1-7, wherein the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample.

9. The method of any one of claims 2-8, wherein the at least one mRNA transcript comprises one or more of the mRNA transcripts of Table A or Table 3.

10. The method of claim 9, wherein the at least one mRNA transcript comprises 2-120, 5-120, 10-120, 50-120, 80-120, 2-128, 5-128, 10-128, 50-128, 80-128, 5-50, 10-50, 50-100, or all of the mRNA transcripts of Table A or Table 3.

11. The method of any one of claims 1-10, wherein the method is performed at least one month, at least two months, at least three months, at least six months, or at least one year after transplantation.

12. The method of any one of claims 1-10, wherein the method is performed from one month to twelve months after transplantation, such as from one month to three months, or from one month to six months after transplantation.

13. The method of any one of claim 2, 3, 4, or 8-12, wherein the expression level of the at least one mRNA transcript is determined by reverse transcription PCR (RT-PCR) (such as quantitative RT-PCR), hybridization to an array, or next generation sequencing.

14. The method of any one of claim 1, or 3-13, wherein the dd-cfDNA level is determined by whole genome sequencing.

15. The method of any one of claim 1 or 3-14, wherein determining the dd-cfDNA level comprises comparison of recipient and donor genotype information.

16. The method of any one of claims 1-10, wherein the dd-cfDNA is determined without comparison to donor genotype information.

17. The method of any one of claim 2, 4, or 8-16, wherein the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects.

18. The method of claim 1 or 3-17, wherein the dd-cfDNA presents a positive predictive value of at least 80%, such as at least 83% or at least 86% and/or a specificity of at least 85%, or at least 90%, or at least 93% for diagnosis of rejection.

19. The method of claim 2 or 4 or 8-18, wherein rejection in the recipient is indicated by result of a trained algorithm based on the expression level of the at least one mRNA transcript indicating rejection or non-rejection, wherein the algorithm compares the expression profile of the at least one mRNA transcript of the recipient to the expression profile of pancreatic transplant subjects with and without rejection, optionally wherein the algorithm has a negative predictive value of at least 80%, such as at least 83% or at least 86% and specificity of at least 75%, such as at least 80% or at least 83%.

20. A method of distinguishing rejection from non-rejection in a pancreatic transplant recipient, the method comprising
a. obtaining a sample from the pancreatic transplant recipient;
b obtaining mRNA from the sample;
c. determining the expression level of at least one mRNA transcript selected from the mRNA transcript of at least one gene listed in Table A or Table 3, wherein the at least one mRNA transcript shows significantly higher expression levels in pancreatic transplant rejection compared to pancreatic transplant non-rejection subjects; and
d. distinguishing rejection from non-rejection in the recipient based upon the expression level of at least one mRNA transcript, optionally wherein rejection in the recipient is indicated by result of a trained algorithm based on the expression level of the at least one mRNA transcript indicating rejection or non-rejection, wherein the algorithm compares the expression profile of the at least one mRNA transcript of the recipient to the expression profile of pancreatic transplant subjects with and without rejection.

21. A method of distinguishing rejection from non-rejection in a pancreatic transplant recipient, the method comprising
a. obtaining a blood, plasma, or serum sample from the pancreatic transplant recipient;
b. obtaining cell-free DNA (cfDNA) and mRNA from the sample;
c. determining (i) the level of donor derived cell-free DNA (dd-cfDNA) in the cfDNA and (ii) the expression level of at least one mRNA transcript selected from the mRNA transcript of at least one gene listed in Table A or Table 3, wherein the at least one mRNA transcript shows significantly higher expression levels in pancreatic transplant rejection compared to pancreatic transplant non-rejection subjects; and
d. distinguishing rejection from non-rejection in the recipient based upon results from both the dd-cfDNA and the expression level of at least one mRNA transcript, wherein rejection in the recipient is indicated by either or both of (i) a level of dd-cfDNA at or above a pre-determined threshold value, and (ii) the expression level of the at least one mRNA transcript.

22. The method of claim 20 or 21, wherein the recipient is a pancreatic and kidney transplant recipient, such as a pancreas after kidney transplant recipient or a simultaneous pancreas and kidney transplant recipient.

23. The method of claim 21 or 22, wherein rejection in the recipient is indicated by predetermined threshold value of dd-cfDNA of ≥0.5%, ≥0.6%, ≥0.7%, ≥0.8%, ≥0.9%, ≥ 1%, ≥1.2%, ≥1.5%, or ≥2%.

24. The method of any one of claims 21-23, wherein rejection in the recipient is indicated by a pre-determined threshold value of dd-cfDNA of ≥0.7%, optionally wherein determining the dd-cfDNA level utilizes data from recipient genotype information.

25. The method of any one of claims 21-23, wherein rejection in the recipient is indicated by a pre-determined threshold value of dd-cfDNA of ≥1.0 or >1.0, optionally wherein determining the dd-cfDNA level utilizes data from recipient genotype information.

26. The method of any one of claims 20-25, wherein the method comprises determining the expression level of 1-2000, 2-2000, 2-500, 10-2000, 20-2000, 10-500, 10-300, 10-200, 100-2000, 100-1000, 100-500, 50-500, 50-300, 50-200, or 100-300 mRNA transcripts in the sample.

27. The method of any one of claims 2-8, wherein the at least one mRNA transcript (a) comprises an mRNA transcript of one or more of: IGHG2, IGHG1, IGHG4, IGHA1, IGLC1, IGHG3, IGKC, IRF4, IL7R, CD96, SLAMF6, SLAMF7, GZMK, IGHM, ZAP70, CD3E, CD79A, CXCR6, CD3D, MIR155HG, CTSW, SLA, IL2RG, CXCR4, ISG20, IL2RB, CCL5, PRDM1, CCR5, CCR2, AOAH, HLA-DQB1, IDO1, GZMA, IKZF1, KLRB1, TNFSF8, CD8A, IL16, HLA-DRA, CD84, BTK, NKG7, SELPLG, CD45R0, PTPRC, or ITGAX; or (b) comprises a group of 2-47, 2-40, 2-30, 2-20, 2-10, 10-40, 10-20, or 20-40 mRNA transcripts listed in Table 3 or selected from IGHG2, IGHG1, IGHG4, IGHA1, IGLC1, IGHG3, IGKC, IRF4, IL7R, CD96, SLAMF6, SLAMF7, GZMK, IGHM, ZAP70, CD3E, CD79A, CXCR6, CD3D, MIR155HG, CTSW, SLA, IL2RG, CXCR4, ISG20, IL2RB, CCL5, PRDM1, CCR5, CCR2, AOAH, HLA-DQB1, IDO1, GZMA, IKZF1, KLRB1, TNFSF8, CD8A, IL16, HLA-DRA, CD84, BTK, NKG7, SELPLG, CD45R0, PTPRC, or ITGAX.

28. The method of any one of claims 20-27, wherein the method is performed at least one month, at least two months, at least three months, at least six months, or at least one year after transplantation.

29. The method of any one of claims 20-27, wherein the method is performed from one month to twelve months after transplantation, such as from one month to three months, or from one month to six months after transplantation.

30. The method of any one of claims 20-29, wherein the expression level of the at least one mRNA transcript is determined by reverse transcription PCR (RT-PCR) (such as quantitative RT-PCR), hybridization to an array, or next generation sequencing.

31. The method of any one of claims 21-29, wherein the dd-cfDNA level is determined by whole genome sequencing.

32. The method of any one of claims 21-29, wherein determining the dd-cfDNA level comprises comparison of recipient and donor genotype information.

33. The method of any one of claims 21-29, wherein the dd-cfDNA is determined without comparison to donor genotype information.

34. The method of any one of claims 20-33, wherein the expression level of the at least one mRNA transcript is normalized against the level of at least one reference mRNA transcript in the sample or against the level of all mRNA in the sample, wherein the at least one reference mRNA transcript does not show significantly different expression levels in transplant rejection compared to non-transplant rejection subjects.

35. The method of any one of claims 1-34, wherein the recipient has one or more of the following characteristics: (a) at least three fold increase in serum lipase and/or serum amylase compared to baseline prior to transplantation, (b) a fasting blood glucose level of >120 mg/dL, (c) presence of donor specific antibodies, or (d) presence of anti-glutamic acid decarboxylase (GAD) antibodies, optionally wherein the method is performed in lieu of a pancreas or kidney biopsy.
